(19) United States
(12) Patent Application Publication
(10) Pub. No.: US 2022/0175893 A1
(43) Pub. Date: Jun. 9, 2022

(54) COMBINATIONS OF TANNASE AND PROBIOTIC FORMULATIONS AND METHODS OF USE FOR IMPROVING TANNIN METABOLISM

(71) Applicant: THE TEXAS A&M UNIVERSITY SYSTEM, College Station, TX (US)

(72) Inventors: Susanne U. TALCOTT, College Station, TX (US); Stephen T. TALCOTT, College Station, TX (US)

(21) Appl. No.: 17/436,021

(22) PCT Filed: Mar. 5, 2020
(86) PCT No.: PCT/US2020/021207

§ 371 Date: Sep. 2, 2021

Related U.S. Application Data
(60) Provisional application No. 62/814,136, filed on Mar. 5, 2019.

(51) Int. Cl. A61K 38/46; A61K 35/747; A61K 35/744; A61K 35/742; A61K 35/741; A61K 31/235; A61K 9/24; A61P 3/02

(52) U.S. Cl. CPC A61K 38/465; A61K 35/747; A61K 35/744; A61P 3/02; A61K 35/741; A61K 31/235; A61K 9/209; A61K 35/742

(57) ABSTRACT

In one aspect, the disclosure relates to pharmaceutical and nutraceutical formulations that overcome inflammatory bowel disease and other disorders or diseases associated with a deficiency in microflora catabolize hydrolyzable tannins Disclosed are methods of using an acid-tolerant tannase that allow the stomach to serve as a “bioreactor” followed by the small intestine for optimal activity, while probiotic strains that specifically target tannins are simultaneously consumed, aiding in the hydrolysis and metabolism. Through colonization, these bacteria can establish and proliferate, and adaption leads to a decrease of the “bad” bacteria while the targeted bacteria proliferate. The formulations and methods provided herein present a short-term (tannase) and long-term (pre- and pro-biotic) solution to poor tannin metabolism. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 62/814,136 filed on Mar. 5, 2019, which is incorporated herein by reference in its entirety.

### TECHNICAL FIELD

The present disclosure generally relates to nutraceutical methods and formulations and uses thereof.

### BACKGROUND

Hydrolyzable tannins are abundant in many fruits, nuts, and botanicals. Products of enzymatic and bacterial catabolism of hydrolyzable tannins are responsible for many health benefits of a plant-based diet, but for individuals that lack the microflora to catabolize these compounds, there is little to no derived benefit of consumption. With some 80 million obese adults and 12 million obese children along with 3-5 million more individuals with some form of inflammatory bowel disease in the US alone, without major changes in dysbiotic gut microflora populations, these individuals will fail to realize the benefits of tannin-containing foods.

There remains a need for methods and formulations that overcome the aforementioned deficiencies.

### SUMMARY

In various aspects pharmaceutical and nutraceutical formulations are provided that overcome one or more of the aforementioned deficiencies. In particular, applicants have found that providing an acid-tolerant tannase will allow the stomach to serve as a “bioreactor” followed by the small intestine for optimal activity, while probiotic strains that specifically target tannins are simultaneously consumed, aiding in the hydrolysis and metabolism. Through colonization, these bacteria will establish and proliferate, and adaption leads to a decrease of the “bad” bacteria while the targeted bacteria proliferate. The formulations and methods provided herein present a short-term (tannase) and long-term (pre- and pro-biotic) solution to poor tannin metabolism.

In various aspects, pharmaceutical or nutraceutical formulations are provided for improving an ability to process dietary tannins in a subject in need thereof. The pharmaceutical or nutraceutical formulations can include an effective amount of a tannin-specific probiotic and an acid-tolerant tannase. For example, the effective amount is effective to improve the ability to of the subject to process dietary tannins as compared to the otherwise same subject consuming the otherwise same amount of dietary tannins except without the pharmaceutical or nutraceutical formulation.

In one aspect, the formulations increase intestinal free gallic acid concentration by at least 50%, 55%, 60%, 65%, 70%, 75%, 80%, 85%, 90%, 95%, 99%, or at least 100%, or a combination of any of the foregoing values, or a range encompassing any of the foregoing values, compared to a subject who has not consumed the pharmaceutical or nutraceutical formulations.

In another aspect, intestinal free gallic acid concentration increases by the disclosed amounts between about 2 hours and about 4 hours of consuming the pharmaceutical or nutraceutical formulations, or in about 2, 2.25, 2.5, 2.75, 3, 3.25, 3.5, 3.75, or about 4 hours after consuming the pharmaceutical or nutraceutical formulations, or a combination of any of the foregoing values, or a range encompassing any of the foregoing values.

The formulations include tannin-specific probiotics. Tannin-specific probiotics aid in the digestion or metabolism of dietary tannins. Examples of tannin-specific probiotics include Lactobacillus plantarum, Lactococcus lactis, Enterococcus faecium and those that encode for 3,4,5-trihydroxybenzoate carboxylase (e.g. Lactobacillus plantarum, Enterobacter aerogenes, Streptococcus gallolyticus, Eubacterium oxidoreducens).

The formulations include one or more acid-tolerant tannases. The acid-tolerant tannase can tolerate the acidic environments of the stomach and provide immediate benefits by generating tannin metabolites from the dietary tannins or from hydrolyzable tannins provided as part of the formulation. Examples of suitable acid-tolerant tannases can include those sourced from members of the Ascochyta, Aspergillus, Penicillium, Fusarium, Trichoderma, Bacillus, Corynebacterium, Lactobacillus, Streptococcus, or Klebsiella genera

In some aspects, applicants have found a benefit of including hydrolyzable tannins in the formulations. The formulations can include hydrolyzable tannins fruits, vegetables, nuts, and botanicals, including, but not limited to, mango, amla, sumac, raspberries, blackberries, blueberries, strawberries, pomegranate, cloudberry, dates, grapefruit, banana, quince, sea buckthorn, apple, grapes, grape seeds, olive, currants, persimmon, gooseberry, cherry, kiwi, avocado, sumac, tea (such as green, oolong, white, black), sage, marjoram, oregano, cloves, chicory, oak, chamomile, peppermint, chestnut, soybeans, walnuts, pecans, walnut, lentils, broad beans, hazelnut, pistachio, and almond.

The formulations can improve the ability to process dietary tannins in the subject, e.g. by at least 30%, at least 40%, at least 60%, at least 80%, at least 100%, or more. Examples of improvements can include one or more of improving the subject's ability to hydrolyze dietary tannins, improving the subject's ability to break down dietary tannins, improving the subject's ability to metabolize dietary tannins, an increase in a blood level of a tannin metabolite in the subject, an increase in a urine level of a tannin metabolite in the subject, and an increase in a fecal tannase activity in the subject.

The formulations can be in a variety of dosage forms such as foods, powders, capsules, and tablets. In some aspects, the dosage is a multi-layered tablet. The multi-layered tablet provides the benefit of providing partially hydrolyzed tannins to the tannin-specific probiotic upon consumption. The multi-layered tablet can include (i) a core containing the tannin-specific probiotic; and (ii) a first layer surrounding the core, wherein the first layer contains the acid-tolerant tannase. In some aspects, the core further includes a prebiotic such as inulin and soluble fibers. In another aspect, the prebiotic can be selected from can be selected from fructans such as, for example, fructooligosaccharides and inulins, galactans such as, for example, galactooligosaccharides, resistant starch, pectin, β-glucans, xylooligosaccharides, mucopolysaccharides, isomaltooligosaccharides, araganogalactans, cellulose ethers, water-soluble hemicellulose, alginates, agar, carrageenan, psyllium, guar gum, gum tragacanth, gum karaya, gum ghatti, gum acacia, gum arabic, combinations thereof, partially hydrolyzed products thereof, and the like. In some aspects, the first layer further includes a hydrolyzable tannin such as those described elsewhere herein. The multi-layered tablet can further include additional layers such as an outer controlled-release coating. In still another aspect, the first layer can also include a hydrolyzable tannin. In any of these aspects, the multi-layer tablet also includes an outer layer surrounding the first layer, wherein the outer layer includes a controlled-release coating.

In one aspect, disclosed herein is a multi-layer tablet having a core that includes a tannin-specific probiotic strain and a first layer surrounding the core, wherein the first layer includes an acid-tolerant tannase. In some aspects, the core further comprises a prebiotic as disclosed herein.

Methods are also provided for improving an ability to process dietary tannins in a subject in need thereof. The methods can include administering an effective amount of a tannin-specific probiotic and an acid-tolerant tannase to the subject; wherein the effective amount is effective to improve the ability of the subject to process dietary tannins as compared to the otherwise same subject consuming the otherwise same amount of dietary tannins except without the pharmaceutical or nutraceutical formulation. The methods can include administering a pharmaceutical or nutraceutical formulation described herein to the subject.

Other systems, methods, features, and advantages of the formulations and methods will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is a graph of the hydrolysis of pentagalloyl glucose (5GG) and subsequent formation of tetragalloyl glucose (4GG), trigalloyl glucose (3GG), digalloyl glucose (2GG) following 2 h incubation with tannase at 10⁻³ U/mL.

FIG. 2 is a chromatogram of five digalloyl glucoses (2GG) and six trigalloyl glucoses (3GG) at 280 nm generated from hydrolysis of pentagalloyl glucose following 2 h incubation with tannase at 10⁻³ U/mL.

FIG. 3 is a graph of hydrolysis of monogalloyl glucose (MGG) and subsequent formation of gallic acid (GA) following 4 h incubation with tannase at 10⁻³ U/mL.

FIG. 4 is a graph of hydrolysis of gallotannin isolate ranging in composition from tetragalloyl glucose (4GG) to undecagalloyl glucose (11GG) following 2 h incubation with tannase at 10⁻³ U/mL.

FIG. 5 is a graph of HPLC chromatogram of blackberry polyphenolics before tannase addition (20 U/mL) showing a diversity of polyphenolics and hydrolyzable tannins. Castalagin is an oligomeric ellagitannin naturally present in the fruit.

FIG. 6 is a graph of HPLC chromatogram of blackberry polyphenolics after tannase addition (20 U/mL) showing a decrease in hydrolyzable tannins and an increase in castalagin, an oligomeric ellagitannin naturally present in the fruit.

FIG. 7 shows the relative fold-increase in free ellagic acid for different fruits over 24 hrs in the presence of tannase (20 U/mL). The production of monometic ellagic acid is less pronounced than the production of oligomers from larger ellagitannin polymers.

FIG. 8 is a bar graph of the hydrolysis of ellagitannins in different fruits over 4 hrs in the presence of tannase (20 U/mL) showing the concentration of free ellagic acid (mg/L) in the presence of processing aids (pecinase and protease) in effort to aid in the hydrolysis of ellagitannins.

FIG. 9 is a quantification of gallic acid (mg/L) during the oral, gastric, and intestinal phases of an in vitro digestion model of sumac after 1 minute of oral incubation with tannase prior to the digestion

FIG. 10 is a quantification of gallic acid (mg/L) during the oral, gastric, and intestinal phases of an in vitro digestion model of sumac after 3 minute of oral incubation with tannase prior to the digestion.

FIG. 11 is a HPLC chromatogram of sumac polyphenolics before tannase addition showing a diversity of polyphenolics and hydrolyzable gallotannins.

FIG. 12 is a HPLC chromatogram of sumac polyphenolics after tannase addition showing the break-down of large tannins into free gallic acid.

FIGS. 13A-13D depict a correlation of pharmacokinetics of polyphenolic metabolites with EMI or plasma biomarkers in lean participants.

FIGS. 14A-14D depict a correlation of pharmacokinetics of polyphenolic metabolites with plasma biomarkers in obese participants.

FIGS. 15A-15D show the polyphenolic production by L. plantarum. HPLC chromatograms of compounds present in L. plantarum cultures incubated with 0.5 mM gallotannins for 24 hours (A and B) and 1.5 mM gallic acid for 2 hours (C and D), respectively.

FIGS. 16A-16B show HPLC chromatogram of compounds present in gallotannin extract and experimental design of the gnotobiotic mouse study. (FIG. 16A) HPLC chromatogram of compounds present in tannin extract at 280 nm. (FIG. 16B) Experimental design of the gnotobiotic mouse study.

FIGS. 17A-171 depict body weight and adiposity of HFD-fed gnotobiotic mice. Changes of body weight of (FIG. 17A) both genders, (FIG. 17B) female, and (FIG. 17C) male in 6 weeks. Epididymal WAT (eWAT) fat mass of (FIG. 17D) both genders, (FIG. 17E) female, and (FIG. 17F) male after 6 weeks. Interscapular BAT (iBAT) fat mass of (FIG. 17G) both genders, (FIG. 17H) female, and (FIG. 17I) male after 6 weeks. n=7. Values are expressed as mean±SEM.

FIGS. 18A-18F depict plasma levels of metabolic hormones and inflammatory cytokines. Fasting plasma levels of (FIG. 18A) glucose, (FIG. 18B) insulin, and (FIG. 18C) HOMA-IR. Adipokines include (FIG. 18D) TNF-α, (FIG. 18E) MCP-1, and (FIG. 18F) leptin after 4 weeks of HFD feeding. n=7. Values are expressed as mean±SEM. Different letters designate significant differences (p<0.05).

FIGS. 19A-19E depict GT and GT with L. plantarum colonization modulated the expressions of molecules involved in lipid metabolism and reduced lipid size in eWAT. Relative mRNA expressions of (FIG. 19A) fat oxidative and lipolytic genes including CPT1, perilipin 1, and HSL, and (FIG. 19B) thermogenic genes including Tmem26, Tbx1, PGC-1α, and Cox2. Protein expressions of (FIG. 19C) FAS, PPARγ, C/EBPα, and β-actin as determined by Western blot. (FIG. 19D) The band intensity in Western blot was determined using ImageJ software. (FIG. 19E) Representative H&E staining for eWAT sections. Values are expressed as mean±SEM. Different letters designate significant differences (p<0.05).

FIGS. 20A-20D depict GT and GT with L. plantarum colonization modulated lipid metabolism and enhanced thermogenesis in iBAT. Relative mRNA expressions of (FIG. 20A) thermogenic genes including Tmem26, Tbx1, PGC-1α, Cox2, PRDM16, and Cox7a1. Protein expressions of (FIG. 20B) p-AMPK, t-AMPK, SIRT1, UCP1, and β-actin as determined by Western blot. (FIG. 20C) The band intensity in Western blot was determined using ImageJ software. (FIG. 20D) Representative H&E staining for iBAT sections. Values are expressed as mean±SEM. Different letters designate significant differences (p<0.05).

FIG. 21 is a schematic diagram of a proposed mechanism for GT with L. plantarum colonization in reducing obesity in gnotobiotic mice. Microbial GT metabolites produced by L. plantarum modulate lipid metabolism by suppressing lipid accumulation through inhibiting the expressions of C/EBPα, PPARγ, and FAS in white adipocytes, as well as promoting thermogenesis through the AMPK-UCP1/SIRT1 axis in brown adipocytes. Adipose tissue secretes lower levels of pro-inflammatory cytokines including TNFα, MCP-1, and leptin. Consequently, the microbial GT metabolites reduce inflammation and improve adipose tissue function and insulin sensitivity in HFD-fed gnotobiotic mice.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure and to be encompassed by the claims herein.

All publications and patents cited in this specification are cited to disclose and describe the methods and/or materials in connection with which the publications are cited. All such publications and patents are herein incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of organic chemistry, pharmacology, and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

The terms “subject” or “patient”, as used herein, refer to any organism to which the active agents and compositions may be administered, e.g. for experimental, therapeutic, diagnostic, and/or prophylactic purposes. Typical subjects include animals (e.g. mammals such as mice, rats, rabbits, non-human primates, and humans).

The terms “sufficient” and “effective”, as used interchangeably herein, refer to an amount (e.g. mass, volume, dosage, concentration, and/or time period) needed to achieve one or more desired result(s).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, “improvement,” “improve,” “improving,” and the like refer to a change in a measurable property such as, for example, a blood level of a tannin metabolite. When assessing improvement, in one aspect, the measurable property can be evaluated by any appropriate means in a subject prior to consuming the tablets, formulations, and/or compositions disclosed herein and one or more times by the same means after consuming the tablets, formulations, and/or compositions disclosed herein, and the values compared. If it is desirable for the value to increase, for example, and the value is shown to increase upon measurement, then improvement has been sufficiently demonstrated. In some aspects, it is desired to know the magnitude of the improvement (e.g., if a property improves by 30% or the like) and this can be calculated by one of ordinary skill in the art after obtaining the initial value prior to administration and a final value after administration.

The term “tannin,” as used herein, refers to astringent, complex phenolic substances of plant origin. Tannins can include phenylpropanoid compounds often condensed to polymers of variable length. Tannins can include hydroxy benzoic acid polymers (namely gallic acid) around a central polyol core, usually glucose. Tannins can include hexahydroxy-diphenic acid and galloylated moieties around a central or multiple polyols, usually glucose. Tannins and are widely distributed secondary metabolites in plants, and play a prominent role in general defense strategies of plants, as well as contributing to food quality. Tannins can have a molecular weight of about 500 g/mol to about 3,000 g/mol or even up to about 20,000 g/mol. The terms ‘hydrolyzable’ and ‘condensed’ tannins are used to distinguish between the two important classes of vegetable tannins, namely gallic acid-derived or ellagic acid-derived versus flavan-3,4-diol-derived tannins, respectively.

Abbreviations: AMPK, AMP-activated protein kinase; ATCC, American Type Culture Collection; BAT, brown adipose tissue; C/EBPα, CCAAT/enhancer binding protein a; Cox2, cyclooxygenase 2; Cox7a1, cytochrome c oxidase 7a1; CPT1, carnitine palmitoyltransferase I; EGCG, epigallolcatechin gallate; eWAT, epididymal white adipose tissue; FAS, fatty acid synthase; GA, gallic acid; GAE, gallic acid equivalent; GF, germ-free; GT, gallotannins; H&E staining, hematoxylin and eosin staining; HbA1c, hemoglobin Mc; HFD, high-fat diet; HOMA-IR, Homeostasis Model Assessment of Insulin Resistance; HPLC-MS, high-performance liquid chromatography-mass spectrometry; HPLC-PDA, high-performance liquid chromatography-photodiode array detector; HSL, hormone-sensitive lipase; iBAT, interscapular brown adipose tissue; IL-8, interleukin 8; LC-ESI-MS, liquid chromatography-electrospray ionization-tandem mass spectrometry; LDL, low-density lipoprotein; MCP-1, monocyte chemoattractant protein-1; OD, optical density; PAI-1, plasminogen activator inhibitor 1; p-AMPKα1, phosphorylated AMPKα1; PBS, phosphate-buffered saline; PG, pyrogallol; PGC1α, peroxisome proliferator-activated receptor γ coactivator 1α; PPARγ, peroxisome proliferator-activated receptor γ; PRDM16, PR domain containing 16; rRNA, ribosomal RNA; SEM, standard error of the mean; SIRT1, Sirtuin1; t-AMPKα1, total-AMPKα1; Tbx1, T-box transcription factor 1; Tmem26, transmembrane protein 26; TNF-α, tumor necrosis factor α; UCP1, uncoupling protein1; VLDL, very low-density lipoprotein; WAT, white adipose tissue.

Formulations

Applicants have found that certain combinations of tannin-specific probiotic and an acid-tolerant tannase can improve the ability of a subject to process dietary tannins. The formulations can include pharmaceutical and nutraceutical formulations capable if improving the ability of a subject to process dietary tannins. Applicants have found that subjects vary greatly in the ability to process dietary tannins, inhibiting some of the expected benefits of a plant-based diet. Applicants have developed the formulations and methods described herein to overcome these problems by improving the ability of the subject to process the dietary tannins.

The formulations and methods can improve the ability to process dietary tannins in the subject, e.g. by at least 30%, 35%, 40%, 45%, 50%, 55%, 60%, 65%, 70%, 75%, 80%, 85%, 90%, 95%, 99%, at least 100%, or more, or a combination of any of the foregoing values, or a range encompassing any of the foregoing values. Examples of improvements can include one or more of improving the subject's ability to hydrolyze dietary tannins, improving the subject's ability to absorb dietary tannins, improving the subject's ability to metabolize dietary tannins, an increase in blood level of a tannin metabolite in the subject, an increase in a urine level of a tannin metabolite in the subject, an increase in a fecal tannase activity in the subject, or a combination thereof.

The pharmaceutical or nutraceutical formulations can include an effective amount of a tannin-specific probiotic and an acid-tolerant tannase. For example, the effective amount is effective to improve the ability to of the subject to process dietary tannins as compared to the otherwise same subject consuming the otherwise same amount of dietary tannins except without the pharmaceutical or nutraceutical formulation.

The formulations include tannin-specific probiotics. Tannin-specific probiotics aid in the digestion or metabolism of dietary tannins. Examples of tannin-specific probiotics include Lactobacillus plantarum, Lactococcus lactis, Enterococcus faecium, and those that encode for 3,4,5-trihydroxybenzoate carboxylase (e.g. Lactobacillus plantarum, Enterobacter aerogenes, Streptococcus gallolyticus, Eubacterium oxidoreducens).

In one aspect, from about 1 to about 10 strains of tannin-specific probiotic bacteria are included in the formulations, or from about 2 to about 6 strains, or from about 2 to about 4 strains, or about 1, 2, 3, 4, 5, 6, 7, 8, 9, or about 1 strains, or a combination of any of the foregoing values, or a range encompassing any of the foregoing values.

In another aspect, each of the strains is present in the composition in a proportion of from about 0.1% to about 99.9%, or from about 1% to about 99%, or from about 10% to about 90%, or at about 0.1, 1, 10, 20, 30, 40, 50, 60, 70, 80, 90, 99, or about 99%, or a combination of any of the foregoing values, or a range encompassing any of the foregoing values.

In one aspect, from about one million to about one trillion colony forming units (CFUs) of each of the tannin-specific probiotic bacteria are included in the formulations. In a further aspect, about 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or about 1×10¹² CFUs of each of the tannin-specific probiotic bacteria are included in the formulations, or a combination of any of the foregoing values, or a range encompassing any of the foregoing values. In one aspect, the same number of CFUs of each strain of tannin-specific probiotic bacteria is included in the formulations. In an alternative aspect, different numbers of CFUs of each strain of tannin-specific probiotic bacteria are included in the formulations.

In one aspect, the tannin-specific probiotic bacteria are provided in a freeze-dried or dormant form. In a further aspect, when the bacteria are freeze-dried or dormant, no additional nutrients are required in the formulations to ensure viability of the bacteria during storage and prior to release. In an alternative aspect, one or more nutrients, minerals, or vitamins can be included in the formulations.

In some aspects, instead of or in addition to the strains of tannin-specific probiotic bacteria as discussed above, the formulations disclosed herein include an isolate or extract from a bacterial culture as disclosed herein. In one aspect, the cells are centrifuged and the isolate or extract is taken from the supernatant of the culture. In another aspect, the cells are filtered such as, for example, by a 0.2 μm filter, and the filtrate forms the isolate or extract. In one aspect, the isolate or extract includes whole cells, lysed cells, or a combination thereof, in addition to some portion of the culture medium. In another aspect, the isolate or extract is further processed such as, for example, by distillation, solvent extraction, or another method prior to inclusion in the formulations.

The formulations include one or more acid-tolerant tannases. The acid-tolerant tannase can tolerate the acidic environments of the stomach and provide immediate benefits by generating tannin metabolites from the dietary tannins or from hydrolyzable tannins provided as part of the formulation. Examples of suitable acid-tolerant tannases can include those sourced from members of the Ascochyta, Aspergillus, Penicillium, Fusarium, Trichoderma, Bacillus, Corynebacterium, Lactobacillus, Streptococcus, or Klebsiella genera.

In one aspect, one unit (U) of tannase activity can be defined as the amount of enzyme that produces 1 μmol of gallic acid per minute under assay conditions as follows: 1% (w/v) methyl gallate is provided as substrate in 100 mM sodium acetate buffer, pH 4.5, with the reaction carried out at 40° C. and gallic acid produced being quantified using methanolic rhodanine as disclosed herein. In one aspect, the formulations disclosed herein contain sufficient quantities of tannase enzyme to achieve from about 1000 to about 25,000 U of tannase activity, or about 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 21,000, 22,000, 23,000, 24,000, or about 25,000 U of tannase activity, or a combination of any of the foregoing values, or a range encompassing any of the foregoing values. In another aspect, the formulations can include from about 5 to about 1000 ppm of a tannase enzyme, or about 5, 10, 25, 50, 75, 100, 150, 200, 250, 30, 350, 400, 450, 500, 550, 600, 650, 700, 750, 800, 850, 900, 950, or about 1000 ppm of a tannase enzyme, or a combination of any of the foregoing values, or a range encompassing any of the foregoing values.

In some aspects, applicants have found a benefit of including hydrolyzable tannins in the formations. The formulations can include hydrolyzable tannins such as those found in grape seed, amla (Indian gooseberry), sumac, berries, pomegranate, or mango. In one aspect, the pharmaceutical and/or nutraceutical formulations disclosed herein include at least one source of hydrolyzable tannins such as, for example, grapes, grape seed, amla, sumac, berries, pomegranate, nuts, mango, or extracts thereof. In a further aspect, the disclosed formulations can include hydrolyzable tannins fruits, vegetables, nuts, and botanicals, including, but not limited to, mango, amla, sumac, raspberries, blackberries, blueberries, strawberries, pomegranate, cloudberry, dates, grapefruit, banana, quince, sea buckthorn, apple, grapes, grape seeds, olive, currants, persimmon, gooseberry, cherry, kiwi, avocado, sumac, tea (such as green, oolong, white, black), sage, marjoram, oregano, cloves, chicory, oak, chamomile, peppermint, chestnut, soybeans, walnuts, pecans, walnut, lentils, broad beans, hazelnut, pistachio, and almond.

In one aspect, the source of hydrolyzable tannins is provided separately from the tannin-specific probiotic strain and the acid-tolerant tannase. In some aspects, the at least one source of hydrolyzable tannins is a food or beverage such as, for example, fresh mango, frozen mango, mango pulp, or a combination thereof. In another aspect, the at least one source of hydrolyzable tannins can be sumac tea. In another aspect, when the source of hydrolyzable tannins is provided separately from the other disclosed components, it can be provided prior to, concurrently with, or after administration of the tannin-specific probiotic strain and acid-tolerant tannase.

In an alternative aspect, the at least one source of hydrolyzable tannins is provided in a single dosage form with the tannin-specific probiotic strain and acid-tolerant tannase.

Applicants have found that, in some aspects, providing the formulations having components in certain ratios provide benefits in improving the effectiveness of the formulations. For examples, in some aspects a ratio (weight/weight) of tannin-specific probiotic to the acid-tolerant tannase is from about 1:1000 to about 100:1, about 1:1000 to about 1:10, about 1:1 to about 1:10, about 1:10 to about 1:40, ora combination of any of the foregoing values or a range encompassing any of the foregoing values. In some aspects, a ratio of the hydrolyzable tannins or source of hydrolyzable tannins to the acid-tolerant tannase is from about 1:1000 to about 100:1, about 1:1000 to about 1:10, about 1:1 to about 1:10, about 1:10 to about 1:40, or a combination of any of the foregoing values or a range encompassing any of the foregoing values.

The formulations can be in a variety of dosage forms such as foods, powders, capsules, and tablets described in detail below. In some aspects, the dosage is a multi-layered tablet. The multi-layered tablet provides the benefit of providing partially hydrolyzed tannins to the tannin-specific probiotic upon consumption. The multi-layered tablet can include (i) a core containing the tannin-specific probiotic; and (ii) a first layer surrounding the core, wherein the first layer contains the acid-tolerant tannase. In some aspects, the core further includes a prebiotic such as inulin and soluble fibers. In another aspect, the prebiotic can be selected from fructans such as, for example, fructooligosaccharides and inulins, galactans such as, for example, galactooligosaccharides, resistant starch, pectin, β-glucans, xylooligosaccharides, mucopolysaccharides, isomaltooligosaccharides, araganogalactans, cellulose ethers, water-soluble hemicellulose, alginates, agar, carrageenan, psyllium, guar gum, gum tragacanth, gum karaya, gum ghatti, gum acacia, gum arabic, combinations thereof, partially hydrolyzed products thereof, and the like. In some aspects, the first layer further includes a hydrolyzable tannin such as those described elsewhere herein. The multi-layered tablet can further include additional layers such as an outer controlled-release coating.

In some aspects a ratio (weight/weight) of tannin-specific probiotic to the acid-tolerant tannase is from about 1:1000 to about 100:1, about 1:1000 to about 1:10, about 1:1 to about 1:10, about 1:10 to about 1:40, or a combination of any of the foregoing values or a range encompassing any of the foregoing values. In some aspects, a ratio of the hydrolyzable tannins or source of hydrolyzable tannins in the multi-layer tablet to the acid-tolerant tannase is from about 1:1000 to about 100:1, about 1:1000 to about 1:10, about 1:1 to about 1:10, about 1:10 to about 1:40, or a combination of any of the foregoing values or a range encompassing any of the foregoing values.

In one aspect, when the formulations contain both a prebiotic and probiotic bacteria, the formulations contain a ratio of from about 1:1000 to about 1:10 of probiotic bacteria to prebiotic material by weight, or of about 1:1000, 1:750, 1:500, 1:250, 1:100, 1:75, 1:50, 1:25, or about 1:10 by weight, or a combination of any of the foregoing values, or a range encompassing any of the foregoing values. In a further aspect, the probiotic bacteria have been cultured with the prebiotic and the entire composition is included in raw or freeze-dried form in the formulations disclosed herein. In an alternative aspect, the probiotic bacteria and prebiotic are added to the formulations separately.

The active agents can be prepared in a variety of oral dosage forms. Suitable oral dosage forms include tablets, capsules, solutions, suspensions, syrups, lozenges, and dry powders. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations are prepared using acceptable carriers. As generally used herein “carrier” includes, but is not limited to, lipids, phospholipids, salts, emulsifiers, excipients, diluents, preservatives, binders, lubricants, disintegrators, swelling agents, fillers, stabilizers, and combinations thereof. Polymers used in the dosage form include hydrophobic or hydrophilic polymers and pH dependent or independent polymers. Preferred hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. Carrier also includes all components of the coating composition, which may include plasticizers, pigments, colorants, stabilizing agents, and glidants.

Formulations can be prepared using one or more excipients, including diluents, preservatives, binders, lubricants, disintegrators, swelling agents, fillers, stabilizers, and combinations thereof.

Controlled release dosage formulations can be prepared as described in standard references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and controlled release dosage forms of tablets, capsules, and granules. These references provide information on carriers, materials, equipment and process for preparing tablets and capsules and controlled release dosage forms of tablets, capsules, and granules.

The active agents may be coated, for example to control release once the particles have passed through the acidic environment of the stomach. Examples of suitable coating materials include, but are not limited to, modified starch or cellulose polymers such as cellulose acetate phthalate, hydroxypropyl cellulose, hydroxypropyl methylcellulose, hydroxypropyl methylcellulose phthalate and hydroxypropyl methylcellulose acetate succinate; lipids such as stearic acid, phospholipids, oils, and the like; cosolvents such as ethanol, glycerin, glycols and water; polyvinyl acetate phthalate, acrylic acid polymers and copolymers, and methacrylic resins that are commercially available under the trade name EUDRAGIT® (Roth Pharma, Westerstadt, Germany), zein, shellac, and other polysaccharides. Additionally, the coating material may contain conventional carriers such as plasticizers, pigments, colorants, glidants, stabilization agents, pore formers and surfactants.

Coatings may be formed with a different ratio of water soluble polymer, water insoluble polymers and/or pH dependent polymers, with or without water insoluble/water soluble non polymeric excipient, to produce the desired release profile. The coating is either performed on dosage form (matrix or simple) which includes, but not limited to, tablets (compressed with or without coated beads), capsules (with or without coated beads), beads, particle compositions, powders, liquids, oils, gels, emulsions, micelles, or liposomes

Optional carriers include, but are not limited to, lipids, phospholipids, salts, emulsifiers, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, VEEGUM® magnesium aluminum silicate, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulfite; cysteine and its derivatives; citric acid; propyl gallate, and butylated hydroxyanisole (BHA).

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid, and hydrogenated vegetable oils.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

Dry Powder Formulations

Dry powder formulations are finely divided solid formulations containing one or more active agents, which are suitable for oral administration. Dry powder formulations can be taken independently or can be added, for instance, to a liquid or food product for ingestion. Dry powder formulations include one or more active agents. Such dry powder formulations can be administered orally to a patient containing one or more active agents. The active agents can be in combination with a pharmaceutically acceptable carrier. In one aspect, the pharmaceutical or nutraceutical composition disclosed herein is provided as a dry powder formulation.

The carrier may include a bulking agent, such as carbohydrates (including monosaccharides, polysaccharides, and cyclodextrins), polypeptides, amino acids, and combinations thereof. Suitable bulking agents include dietary fiber, fructose, galactose, glucose, lactitol, lactose, maltitol, maltose, maltodextrin, mannitol, starches, sucrose, trehalose, xylitol, hydrates thereof, and combinations thereof. The pharmaceutical carrier may include any of those previously stated.

In any of the above aspects, also disclosed is a method for improving an ability to process dietary tannins in a subject in need thereof, the method including administering the pharmaceutical or nutraceutical composition disclosed herein or the multi-layer tablet disclosed herein, to the subject.

### Aspects

The following listing of exemplary aspects supports and is supported by the disclosure provided herein.

Aspect 1. A pharmaceutical or nutraceutical formulation for improving an ability to process dietary tannins in a subject in need thereof, the formulation comprising an effective amount of a tannin-specific probiotic strain and an acid-tolerant tannase.

Aspect 2. The pharmaceutical or nutraceutical formulation of aspect 1, wherein the formulation increases intestinal free gallic acid concentration by at least 50%.

Aspect 3. The pharmaceutical or nutraceutical formulation of aspect 1, wherein the formulation increases intestinal free gallic acid concentration by at least 75%.

Aspect 4. The pharmaceutical or nutraceutical formulation of aspect 1, wherein the formulation increases intestinal free gallic acid concentration by at least 100%.

Aspect 5. The pharmaceutical or nutraceutical formulation of any of aspects 2-4, wherein intestinal free gallic acid concentration increases between about 2 hours and about 4 hours of administering the formulation to the subject.

Aspect 6. The pharmaceutical or nutraceutical formulation of any of aspects 1-5, wherein the tannin-specific probiotic strain comprises Lactobacillus plantarum, Lactococcus lactis, Enterococcus faecium, Enterobacter aerogenes, Streptococcus gallolyticus, Eubacterium oxidoreducens, or a combination thereof.

Aspect 7. The pharmaceutical or nutraceutical formulation of any of aspects 1-6, wherein the acid-tolerant tannase is sourced from an Ascochyta species, an Aspergillus species, a Penicillium species, a Fusarium species, a Trichoderma species, a Bacillus species, a Corynebacterium species, a Lactobacillus species, a Streptococcus species, a Klebsiella species, or a combination thereof.

Aspect 8. The pharmaceutical or nutraceutical formulation of aspect 7, wherein the ratio (w/w) of tannin-specific probiotic strain to the acid-tolerant tannase is from about 1:1000 to about 100:1.

Aspect 9. The pharmaceutical or nutraceutical formulation of aspect 7, wherein the ratio (w/w) of tannin-specific probiotic strain to the acid-tolerant tannase is from about 1:1000 to about 1:10.

Aspect 10. The pharmaceutical or nutraceutical formulation of aspect 7, wherein the ratio (w/w) of tannin-specific probiotic strain to the acid-tolerant tannase is from about 1:1 to about 1:10.

Aspect 11. The pharmaceutical or nutraceutical formulation of any of aspects 1-10, further comprising at least one source of hydrolyzable tannins.

Aspect 12. The pharmaceutical or nutraceutical formulation of aspect 11, wherein the at least one source of hydrolyzable tannins comprises grapes, grape seed, amla, sumac, berries, pomegranate, nuts, mango, or extracts thereof.

Aspect 13. The pharmaceutical or nutraceutical formulation of aspect 12, wherein the ratio (w/w) of the source of hydrolyzable tannin to the acid-tolerant tannase is from about 1:1000 to about 100:1.

Aspect 14. The pharmaceutical or nutraceutical formulation of aspect 12, wherein the ratio (w/w) of the source of hydrolyzable tannin to the acid-tolerant tannase is from about 1:1000 to about 1:10.

Aspect 15. The pharmaceutical or nutraceutical formulation of aspect 12, wherein the ratio (w/w) of the source of hydrolyzable tannin to the acid-tolerant tannase is from about 1:1 to about 1:10.

Aspect 16. The pharmaceutical or nutraceutical formulation of any of aspects 1-15, wherein improving the ability to process dietary tannins comprises improving the subject's ability to hydrolyze dietary tannins, improving the subject's ability to absorb dietary tannins, improving the subject's ability to metabolize dietary tannins, increasing a urine level of a tannin metabolite in the subject, increasing a fecal level of a tannin metabolite in the subject, increasing a blood level of a tannin metabolite in the subject, or a combination thereof.

Aspect 17. The pharmaceutical or nutraceutical formulation of aspect 16, wherein improving the ability to process dietary tannins comprises an improvement of at least 30%.

Aspect 18. The pharmaceutical or nutraceutical formulation of aspect 16, wherein improving the ability to process dietary tannins comprises an improvement of at least 50%.

Aspect 19. The pharmaceutical or nutraceutical formulation of aspect 16, wherein improving the ability to process dietary tannins comprises an improvement of at least 100%.

Aspect 20. The pharmaceutical or nutraceutical formulation of any of aspects 11-19, wherein the at least one source of hydrolyzable tannins is provided separately from the tannin-specific probiotic strain and the acid-tolerant tannase.

Aspect 21. The pharmaceutical or nutraceutical formulation of aspect 20, wherein the at least one source of hydrolyzable tannins comprises a food or beverage.

Aspect 22. The pharmaceutical or nutraceutical formulation of aspect 21, wherein the at least one source of hydrolyzable tannins comprises fresh mango, frozen mango, mango pulp, or a combination thereof.

Aspect 23. The pharmaceutical or nutraceutical formulation of aspect 21, wherein the at least one source of hydrolyzable tannins comprises sumac tea.

Aspect 24. The pharmaceutical or nutraceutical formulation of any of aspects 20-24, wherein the at least one source of hydrolyzable tannins is consumed prior to administration of the tannin-specific probiotic strain and acid-tolerant tannase.

Aspect 25. The pharmaceutical or nutraceutical formulation of any of aspects 20-24, wherein the at least one source of hydrolyzable tannins is consumed concurrently with administration of the tannin-specific probiotic strain and acid-tolerant tannase.

Aspect 26. The pharmaceutical or nutraceutical formulation of any of aspects 20-24, wherein the at least one source of hydrolyzable tannins is consumed after administration of the tannin-specific probiotic strain and acid-tolerant tannase.

Aspect 27. The pharmaceutical or nutraceutical formulation of any of aspects 11-19, wherein the at least one source of hydrolyzable tannins is provided in a single dosage form with the tannin-specific probiotic strain and the acid-tolerant tannase.

Aspect 28. The pharmaceutical or nutraceutical formulation of any of aspects 1-19 or 27, wherein the formulation comprises a powder formulation.

Aspect 29. The pharmaceutical or nutraceutical formulation of any of aspects 1-19 or 27, wherein the formulation comprises a tablet or capsule.

Aspect 30. The pharmaceutical or nutraceutical formulation of any of aspects 1-29, further comprising a prebiotic.

Aspect 31. The pharmaceutical or nutraceutical formulation of aspect 30, wherein the prebiotic comprises a fructooligosaccharide, inulin, a galactooligosaccharide, resistant starch, pectin, a β-glucan, a xylooligosaccharide, a mucopolysaccharide, an isomaltooligosaccharide, an araganogalactan, a cellulose ether, a water-soluble hemicellulose, an alginate, agar, carrageenan, psyllium, guar gum, gum tragacanth, gum karaya, gum ghatti, gum acacia, gum arabic, a combination thereof, or a partially-hydrolyzed product thereof.

Aspect 32. The pharmaceutical or nutraceutical formulation of aspect 30, wherein the prebiotic comprises inulin.

Aspect 33. A multi-layer tablet comprising: (a) a core comprising a tannin-specific probiotic strain; and (b) a first layer surrounding the core, wherein the first layer comprises an acid-tolerant tannase.

Aspect 34. The multi-layer tablet of aspect 33, the core further comprising a prebiotic.

Aspect 35. The multi-layer tablet of aspect 33, the first layer further comprising a hydrolyzable tannin.

Aspect 36. The multi-layer tablet of any of aspects 33-35, further comprising an outer layer surrounding the first layer, wherein the outer layer comprises a controlled-release coating.

Aspect 37. The multi-layer tablet of any of aspects 33-36, wherein the ratio (w/w) of tannin-specific probiotic strain to the acid-tolerant tannase is from about 1:1000 to about 100:1.

Aspect 38. The multi-layer tablet of any of aspects 33-36, wherein the ratio (w/w) of tannin-specific probiotic strain to the acid-tolerant tannase is from about 1:1000 to about 1:10.

Aspect 39. The multi-layer tablet of any of aspects 33-36, wherein the ratio (w/w) of tannin-specific probiotic strain to the acid-tolerant tannase is from about 1:1 to about 1:10.

Aspect 40. The multi-layer tablet of any of aspects 35-39, wherein the ratio (w/w) of the hydrolyzable tannin to the acid-tolerant tannase is from about 1:1000 to about 100:1.

Aspect 41. The multi-layer tablet of any of aspects 35-39, wherein the ratio (w/w) of the hydrolyzable tannin to the acid-tolerant tannase is from about 1:1000 to about 1:10.

Aspect 42. The multi-layer tablet of any of aspects 35-39, wherein the ratio (w/w) of the hydrolyzable tannin to the acid-tolerant tannase is from about 1:1 to about 1:10.

Aspect 43. A method for improving an ability to process dietary tannins in a subject in need thereof, the method comprising administering the pharmaceutical or nutraceutical composition of any of aspects 1-32 or the multi-layer tablet of any of aspects 33-42 to the subject.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1: Gallotannin and Mango Gallotannin Hydrolysis

Gallic acid glycosides and other gallated species are reservoirs of bioactive gallic acid, and have the potential to be hydrolyzed to free gallic acid through both enzymatic and non-enzymatic hydrolysis. Gallic acid is reported to have both anti-cancer and anti-inflammatory properties, and quantification of its total gallic acid content in gallic acid polymers is paramount. Therefore, a new method to rapidly quantify the total gallic acid content of galloyl glycosides using tannase as a hydrolysis aid was developed, and after 1 h of incubation with 20 U/mL of tannase monogalloyl glucose and large gallotannins were completely hydrolyzed to free gallic acid. This new method will allow for rapid quantification pro-gallic acid content in the many foods that contain gallic acid glycosides. In addition, characterization of hydrolysis of pentagalloyl glucose led to the development of two tetragalloyl glucoses, six trigalloyl glucoses, and five digalloyl glucoses each hypothesized to have unique stereochemistry.

The link between the consumption of fruits and vegetables and the prevention of cancer and cardiovascular diseases has been extensively reviewed. (Bradbury, Appleby, & Key, 2014; Wang et al, 2014), and has largely been attributed to their polyphenols, a class of phytochemicals found in higher plants that have a polyaromatic core attached with multiple hydroxyl groups. As a result, understanding the mechanisms behind polyphenols preventative effects is currently of great scientific inquiry. Due to the diversity in chemical structures within the polyphenol class, it is important to individually investigate each different compound to understand underlying mechanisms. Tannins are a class of polyphenols found in a wide array of fruits, vegetables, and legumes that have been used for centuries as preservation agents in the process of turning hides into leather, and are additionally known for their ability to form complexes with protein and cause astringency in foods (Serrano et al., 2009). Tannins are categorized into two subtypes, condensed and hydrolyzable. Condensed tannins are oligomers of flavan-3-ols that are linked carbon to carbon via an interflavan bond. The hydrolyzable tannins are further divided into gallotannins and ellagitannins, while complex tannins are a further sub-class that contain both gallic acid and ellagic acid upon hydrolysis. Gallotannins are polymers of gallic acid esterified to a polyol core, and ellagitannins are made from both gallic acid and hexahydroxydephenic acid moieties esterified to a polyol. In contrast to condensed tannins, hydrolyzable tannins are polymerized through esterification of the monomeric units, and these ester bonds are capable of being hydrolyzed through both enzymatic and non-enzymatic means (Krook and Hagerman, 2012; Rodriguez et al., 2008). Historically, tannins have been difficult to analyze due to poor separation even with high performance liquid chromatography, however, some successful attempts have recently been made (Newsome, Li, & van Breeman, 2016).

Tannase (tannin acyl hydrolase) is an enzyme capable of hydrolyzing ester and depside bonds of hydrolyzable tannins (Belmares et al., 2004). Tannase is produced by several genera of fungi including but not limited to Aspergillus, Penicillium, Fusarium, Trichoderma and bacteria, Lactobacillus spp and Streptococcus gallolyticus, and commercially is used to prevent creaming in instant tea, clarification of beer and fruit juice, and to produce acorn wine and coffee flavored beverages (Aguilar et al., 2007; Yao, Guo, Ren, & Liu, 2014). Recent interest has been given to the tannase produced in the colons of individuals and its potential impacts on the digestion of foods, mainly through the release of gallic acid, which has been shown to possess anti-cancer and anti-inflammatory properties (Kaur et al., 2009; Kawada et al., 2001). However, inter-individual differences in tannase activity will affect the rate at which gallic acid is hydrolyzed and the efficiency of hydrolysis in vivo is unknown. The production of gallic acid by hydrolysis from tannase has been monitored in numerous studies; however, the characterization of the intermediates of tannase hydrolysis, specifically the hydrolysis of pentagalloyl glucose, has not yet been investigated.

In these studies, the enzymatic hydrolysis with tannase was performed on monogalloyl glucose, pentagalloyl glucose, and a gallotannin isolate extracted from mango, and hydrolysates were characterized and quantified over time. An application for tannase was additionally investigated for use as a tool to rapidly analyze total gallic acid content of gallotannins. Mangoes were sourced from Mexico through Frontera produce, and were allowed to ripen at room temperature. Fruits were manually peeled, deseeded, and vacuum sealed under good manufacturing practices. Mango pulp was held at −20° C. until used for experiments. Polyphenol extracts from mango were prepared from 1 kg of mango pulp extracted with 2 L of a 1:1 ratio of acetone and methanol in triplicate. Solvents were pooled, evaporated under vacuum at 45° C., and brought up to a known volume of water acidified with 0.1 M HCl. An isolate consisting of only gallotannins was prepared from Ataulfo mango pulp and isolated as previously described by Hagerman (2011). Briefly, 500 mL of reagent alcohol was added to 500 mL of the polyphenol mango extract, and loaded on to a column filled with 20 g of Sephadex LH-20 that was previously conditioned with 20 column volumes of reagent alcohol. Once the sample was loaded tannins were eluted using 80% acetone in water acidified with 0.1% formic acid. The eluted gallotannin isolate was evaporated under vacuum, and stored at −20° C. until use.

Standards solutions of monogalloyl glucose, pentagalloyl glucose, and a gallotannin isolate were incubated with tannase at 10⁻³ U/mL to characterize the by-products and relative rates of galloyl derivative hydrolysis. 250 μL of 200 ppm monogalloyl glucose or pentagalloyl glucose were incubated with 650 μL of buffer set to the enzyme's optimum conditions (pH 5.5, 0.1 M citric acid buffer, 30° C.), and 100 μL of tannase 10⁻³ U/mL for final standard concentrations of 50 ppm. For the gallotannin isolate, 250 μL of a 969±31 ppm gallic acid equivalents (GAE) as determined by the Folin-Ciocalteu method was used (Singleton et al, 1965). Experiments were performed in a static water bath and prepared in triplicate for each time point at 0, 0.016, 0.033, 0.050, 0.083, 0.167, 0.5, 1, 1.5, 2, 3, 4 h. To end enzymatic activity, solutions were immediately diluted with 1000 μL 0.1% formic acid MeOH. Lastly, samples were filtered with a 0.45 μm syringe filter prior to LC-MS analysis.

200 uL of 641±16 and 2051±46 ppm GAE gallotannin isolate was aliquotted into test tubes and made up to 1 mL with 20 U/mL tannase hydrated in pH 5.5, 0.1 M citrate buffer. Samples were incubated for 0, 0.16 (10 min), 0.5, 1, 2, 4, 6, 12, and 24 h at 35° C. in a static water bath. After each time point had elapsed, 1 mL of 0.1% formic acid MeOH was added to each test tube. 50 and 100 ppm monogalloyl glucose was also treated with tannase under the same conditions. The total pro-gallic acid content of the gallotannin isolate and monogalloyl glucose were quantified by the colorimetric rhodanine assay modified from Inoue & Hagerman (1988). 600 μL of 0.667% rhodanine in methanol was added to 400 μL of hydrolyzed tannin fraction sample. After 10 min, 400 μL of 0.5 M NaOH was added to develop color for 20 min until bringing the final volume to 10 mL with deionized water. Absorbance was measured at 520 nm.

Galloyl derivatives were characterized and quantified by use of LC-MS on a Thermo Finnigan HPLC. Separations were in reversed-phase using a Finnigan Surveyor HPLC coupled to a Surveyor PDA detector and gradient separations were performed using a Phenomenex Kinetex™ (Bannockburn, II) C₁₈ column, (150×4.6 mm, 2.6 μm) at room temperature. Injections were made into the column by use of a 50 μL sample loop. For separation of gallic acid and galloyl glycosides mobile phase A was 0.1% formic acid in water and mobile phase B was 0.1% formic acid in methanol run at 0.45 mL/min. A gradient was run of 0% Phase B for 2 min and changed to 10% Phase B in 4 min, 10% Phase B was held to 10 min, 10 to 40% Phase B in 25 min, and 40% to 65% Phase B in 35 min, 65% to 85% Phase B in 41 min, 85% was held to 49 min before returning to initial conditions. The electrospray interface worked in negative ionization mode. Source and capillary temperatures were set at 325° C., source voltage was 4.0 kV, capillary voltage was set at −47 V, and collision energy for MS/MS analysis was set at 35 eV. The instrument operated with sheath gas and auxiliary gas (N₂) flow rates set at 10 units/min and 5 units/min, respectively. The instrument was tuned specifically for pentagalloyl glucose. Gallic acid, mono galloyl glucose were quantified at 280 nm with their respective standards. Digalloyl glucoses, and trigalloyl glucoses were quantified at 280 nm and reported as equivalents of monogalloyl glucose. Tetragalloyl glucose and higher were quantified and reported as pentagalloyl glucose equivalents.

Tannase sourced from Aspergillus oryzae was incubated with standard solutions of monogalloyl glucose, pentagalloyl glucose, and a gallotannin isolate sourced from mango in an effort to characterize and quantify the intermediates formed during hydrolysis. Prior investigations on tannase hydrolysis have focused primarily on the substrates: methyl gallate, propyl gallate, epigallocatechin-gallate, and tannic acid, and specifically only on the rate gallic acid production (Battestin, Macedo, and De Freitas, 2008; Chang et al., 2006). Characterization of the different galloyl derivatives that can be formed during tannase hydrolysis is critical as they are likely to have different bio efficacies.

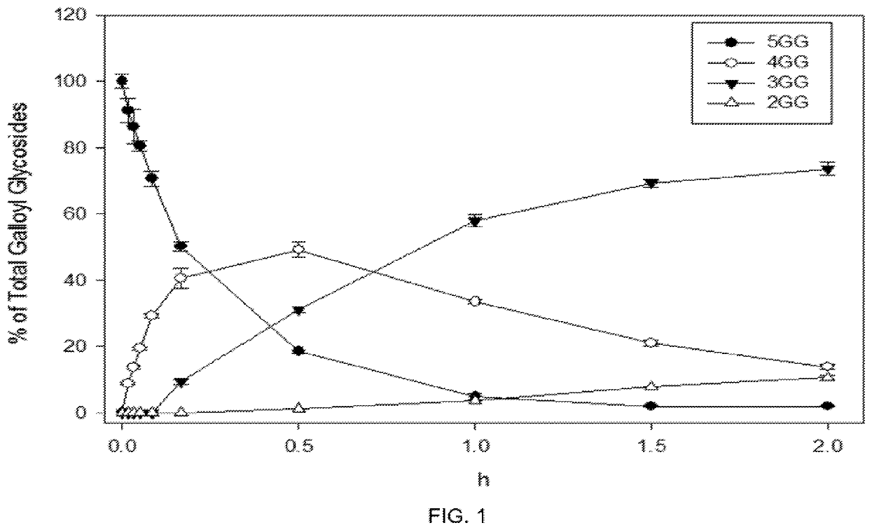

Hydrolysis of pentagalloyl glucose and other galloyl glycosides with tannase is unique compared to most other enzymatic reactions as additional substrate is still being generated upon hydrolysis, similar to the hydrolysis of glucose from maltodextrins with amylases. In this study, after 2 h incubation of pentagalloyl glucose with tannase at 10⁻³ U/mL, 97.2±0.27% of all the pentagalloyl glucose had been hydrolyzed gallic acid to create smaller galloyl glycosides (FIG. 1). In 2 h, two tetragalloyl glucoses, six trigalloyl glucoses, and five digalloyl glucoses were characterized from m/z previously reported by (Berardini, Carle, & Schieber, 2004). Specifically, tetragallyol glucose was characterized from a parent ion at m/z 787 and fragment ions at m/z 635, 465 corresponding to the losses of galloyl moieties. Trigalloyl glucose at m/z 635 and fragments at m/z 483, 422, and 313, and digalloyl glucose was characterized from a parent ion at m/z 483 and fragment ions at m/z 331, 271, and 169.

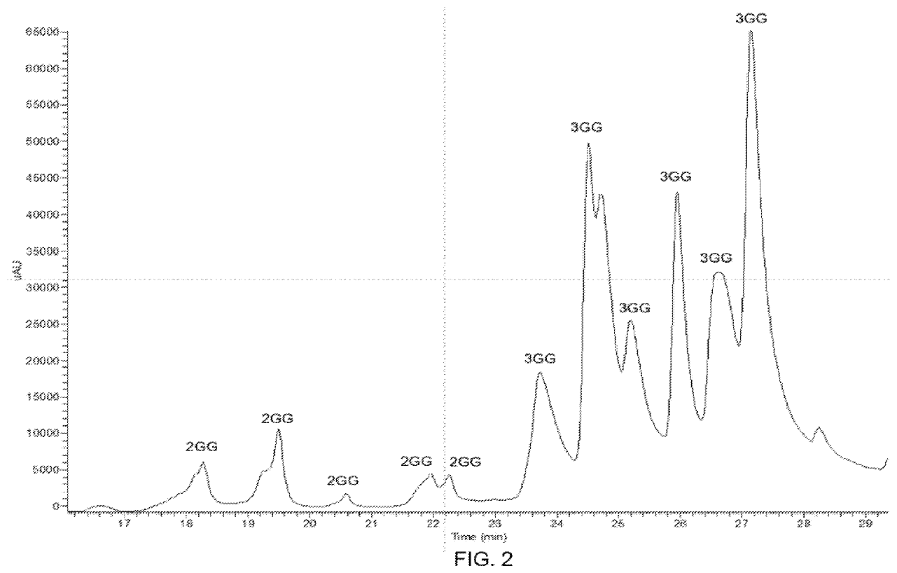

At 0.5 h, the combined tetragalloyl glucoses reached a maximum concentration of 49.2±2.22% of the total galloyl glycosides content, but at 2 h decreased to 13.8±0.68%. Additionally at 2 h, digalloyl glucoses and trigalloylglucses were still being generated making up 10.4±0.65 and 73.7±1.92% of the total galloyl glycosides content, respectively. The six trigalloyl glucoses and five digalloyl glucoses each had distinct retention times (FIG. 2), and from this are hypothesized to each have unique stereochemistry. This is in contrast to the synthesis of pentagalloyl glucose which follows a specific enzymatic pathway where condensation reactions add additional galloyl groups in a stepwise manner (Niemetz & Gross, 2005). The presence of distinct di and trigalloyl glycosides shows tannase hydrolysis can occur at any position. Digalloyl and trigalloylglucoses can also be found in plants that contain gallotannins as they are necessary for the formation of larger tannins, however, they are only found in two possible positions, 1,2,6-trigalloylglucose and 1,3,6-trigalloyl glucose (Gross et al., 1993). The different rates of tannase hydrolysis per person could impact the amount of these different digalloyl and trigalloyl in the lumen which has potential to affect their bioefficacy.

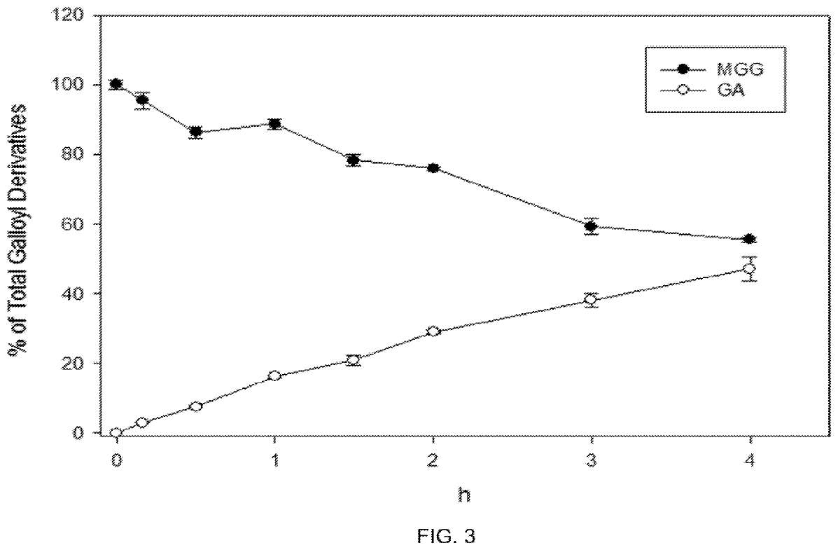

On a molar basis 45.5±0.53% of the total pro-gallic acid content was generated from hydrolysis of pentagalloyl glucose following 2 h of incubation. When an equivalent amount monogalloyl glucose was incubated with the same duration and tannase activity only 28.9±0.26% was generated, a significant (p<0.05) difference (FIG. 3). It took a total of 4 h to reach the same amount of generated gallic acid. At the 20 mg/L investigated here there is more monogalloyl glucose on a molar level than pentagalloyl glucose, but when compared it still took longer to generate the same moles of gallic acid. This difference could be due to different enzymatic activities of different galloyl-glucose bonds. Wu et al. have previously demonstrated that tannase has different activities for different substrates (2015).

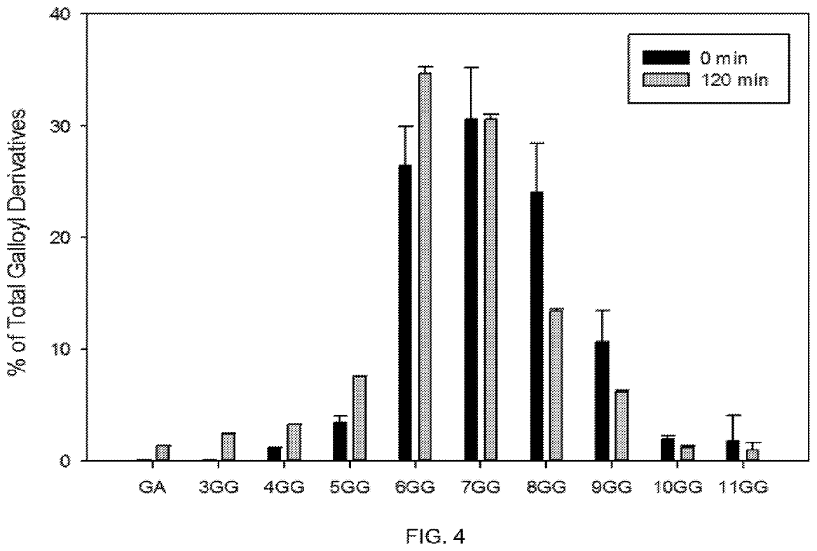

A mango gallotannin isolate containing tannins ranging in degree of polymerization from pentagalloyl glucose to undecagalloyl glucose was additionally incubated with tannase at 10⁻³ U/mL (FIG. 4). After 2 h there was still quantifiable amount of all gallotannins, and significant increases in gallic acid, trigalloyl glucoses, tetra, galloyl glucoses, and pentagalloyl glucose were observed. Additionally, no digallic acid or trigallic acid were characterized whose presence would indicate direct hydrolysis of galloyl-glucose bonds of the larger gallotannins instead of just the depside bonds connecting to gallic acids together. Ren et al. (2013) have previously described the structure and binding site of galloyl in tannase produced by Lactobacillus plantarum, and found that there was only one binding site for both depside and esterase activities, and that only the single galloyl moiety enters the binding site, which would explain the lack of digallic acid and trigallic acid.

An improved method for analyzing the total gallic acid content of galloyl glycosides from hydrolysis with tannase was evaluated using monogalloyl glucose and a gallotannin isolate. Inoue & Hagerman (1988) found that rhodanine (2-thioxo-4-thiazolidinone) forms a red complex with free gallic acid whose absorbance at 520 nm linearly correlates to the concentration of free gallic acid, and measured the total pro-gallic acid content from gallotannins present in leaf samples by digesting in an excess of sulfuric acid for 26 h. Nakamura et al. (2003) utilized 2500 U/mL tannase to completely hydrolyze tannic acid into gallic acid, but quantified gallic acid using HPLC. It was found here that a minimal amount of tannase (20 U/mL) can hydrolyze gallates and galloyl glycosides into free gallic acid as to completion more rapidly than acid hydrolysis. After 1 h, there was no significant difference (p<0.05) in the concentration of free gallic acid post enzymatic hydrolysis for both concentrations of gallotannin isolate in comparison to later time points (Table 1). This indicated that complete hydrolysis of mango gallotannins was achieved, which was also confirmed by LC-MS analysis by the lack of galloyl ester hydrolysis product ions detected (data not shown). Complete hydrolysis of gallotannins to gallic acid is imperative to prevent underestimation of total pro-gallic acid content as rhodanine does not form a complex with gallic acid esters, including monogalloyl glucose (Inoue & Hagerman, 1988). Although not significantly different, the concentration of free gallic acid after 24 h was slightly lower. Gallic acid irreversibly degrades in basic conditions and hydrolysis for this method was conducted in a pH 5.5 buffer, the optimal pH of the commercial tannase (Friedman & Jurgens, 2000). However, gallic acid was shown to be stable at the conditions of the method as 50 and 100 ppm gallic acid in pH 5.5 buffer did not show any indication of degradation after 24 h.

TABLE 1
Stability of Gallic Acid at pH 5.5 over 24 h, and Hydrolysis of Monogalloyl glucose and a Gallotannin Isolate with 20
U/mL of tannase at pH 5.5 over 24.¹

| | Gallic Acid | | Monogalloyl Glucose | | Gallotannin Isolate | |
|---|---|---|---|---|---|---|
| Time | 50 | 100 | 50 | 100 | 600 | 2000 |
| (h) | (mg/L) | (mg/L) | (mg/L) | (mg/L) | (mg GAE/L) | (mg GAE/L) |
| 0 | 48.2 ± 1.28 ^{a} | 107 ± 3.07 ^{a} | 1.26 ± 0.13 ^{a} | 1.76 ± 0.25 ^{a} | 28.3 ± 0.00 ^{a} | 81.8 ± 6.29 ^{a} |
| 1 | 50.4 ± 0.45 ^{b} | 102 ± 2.43 ^{b} | 19.9 ± 0.13 ^{b} | 39.7 ± 0.50 ^{b} | 522 ± 11.3 ^{b} | 1680 ± 35.1 ^{b} |
| 2 | 51.2 ± 0.62 ^{b} | 111 ± 1.63 ^{b} | 20.2 ± 0.88 ^{b} | 42.5 ± 1.01 ^{b} | 516 ± 20.6 ^{b} | 1840 ± 38.2 ^{b} |
| 12 | 47.2 ± 0.87 ^{b} | 97.9 ± 1.38 ^{b} | 20.1 ± 0.25 ^{b} | 39.2 ± 1.89 ^{b} | 525 ± 19.1 ^{b} | 1680 ± 28.8 ^{b} |
| 24 | 49.4 ± 0.37 ^{b} | 108 ± 1.11 ^{b} | 19.9 ± 0.87 ^{b} | 35.7 ± 3.14 ^{b} | 471 ± 14.4 ^{b} | 1580 ± 68.0 ^{b} |

¹ Different letters within the same column signify a significant difference.

Monogalloyl glucose is the precursor for the synthesis of gallotannins in higher plants and therefore can be naturally present in gallotannin containing plants (Niemetz & Gross, 2005). As previously discussed, monogalloyl glucose is hydrolyzed by tannase at a slower rate than gallotannins. For this reason and for the first time the ability of 20 U/mL of tannase to completely hydrolyze monogalloyl glucose was assessed. 50 and 100 ppm solutions of monogalloyl glucose were hydrolyzed with 20 U/mL Tannase and after 1 h there were no significant differences (p<0.05) between concentrations of gallic acid produced at later time points for both concentrations. The complete hydrolysis of monogalloyl glucose was confirmed on HPLC with no detectable peaks of monogalloyl glucose. The ability of tannase to completely hydrolyze high concentrations of gallotannins and monogalloyl glucose within 1 h and the stability of gallic acid in the conditions of the method demonstrates the reliability of tannase hydrolysis to accurately and rapidly determine the pro-gallic acid content of galloyl glycosides.

Other than mango, gallic acid and gallic acid derivatives are naturally present in a variety of foods, beverages, and herbs/spices including galloylated flavan-3-ols in green tea, gallotannins in chickpea, cow peas, persimmons, star fruit, pecans, and sumac, and galloylated ellagitannins in berries (Hager et al., 2008; Lu et al., 2009; Serrano et al., 2009). Gallic acid has also been shown to be bioavailable in humans and potentially have anti-mutagenic, anti-inflammatory, and neuroprotective properties (Shahrzad et al., 2001). More emphasis has been put on understanding the possible biological benefits and metabolism of polyphenols and their resulting catabolites (Scalbert et al., 2002). For example, 4-O-methyl-gallic acid-3-O-sulfate and pyrogallol have been identified in human plasma and urine and are believed to be metabolites derived from gallic acid (Pimpao et al., 2014). Gallic acid derived metabolites do not result from solely free gallic acid but also larger galloylated polyphenols such as (−)-epigallocatechin-3-O-gallate (Van der Pijl et al., 2015) and gallotannins. The method presented here can help determine the quantity of both free and esterified gallic acid in a food substance that can be potentially absorbed or metabolized post-consumption.

In summary, a new method for the measurement of pro-gallic acid content from enzymatic hydrolysis of gallic acid glycosides with tannase was developed and presented herein. Additionally, the intermediates from hydrolysis were evaluated and when pentagalloyl glucose was hydrolyzed two tetragalloyl glucoses, six trigalloyl glucoses, and five digalloyl glucoses each hypothesized to have unique stereochemistry. Galloyl glycosides and other gallated species are found in many foods stuffs and have the potential to be consumed by a large population. This new method will allow for efficient and rapid analysis of the pro-gallic acid content of foods that contain gallic acid glycosides.

### References for Example 1

1. Aguilar, C. N., Rodriguez, R., Gutierrez-Sánchez, G., Augur, C., Favela-Torres, E.,
Prado-Barragan, L. A., Ramirez-Coronel, A., & Contreras-Esquivel, J. C. (2007). Microbial tannases: advances and perspectives. Applied Microbiology and Biotechnology, 76(1), 47-59.
2. Battestin, V., Macedo, G. A., & De Freitas, V. A. P. (2008). Hydrolysis of epigallocatechin gallate using a tannase from Paecilomyces variotii. Food Chemistry, 108(1), 228-233.
3. Belmares, R., Contreras-Esquivel, J. C., Rodriguez-Herrera, R., Coronel, A. R. r., & Aguilar, C. N. (2004). Microbial production of tannase: an enzyme with potential use in food industry. LVVT—Food Science and Technology, 37(8), 857-864.
4. Berardini, N., Carle, R., & Schieber, A. (2004). Characterization of gallotannins and benzophenone derivatives from mango (Mangifera indica L. cv. ‘Tommy Atkins’) peels, pulp and kernels by high-performance liquid chromatography/electrospray ionization mass spectrometry. Rapid Communications in Mass Spectrometry, 18(19), 2208-2216.
5. Bradbury, K. E., Appleby, P. N., & Key, T. J. (2014). Fruit, vegetable, and fiber intake in relation to cancer risk: findings from the European Prospective Investigation into Cancer and Nutrition (EPIC). The American Journal of Clinical Nutrition, 100(Supplement 1), 394S-398S.
6. Chang, F.-S., Chen, P.-C., Chen, R. L. C., Lu, F.-M., & Cheng, T.-J. (2006). Real-time assay of immobilized tannase with a stopped-flow conductometric device. Bioelectrochemistry, 69(1), 113-116.
7. Friedman, M., & Jurgens, H. S. (2000). Effect of pH on the stability of plant phenolic compounds. Journal of Agriculture and Food Chemistry, 48, 2101-2110.
8. Hager, T. J., Howard, L. R., Liyanage, R., Lay, J. O., Prior, R. L. (2008). Ellagitannin composition of blackberry as determined by HPLC-ESI-MS and MALDI-TOF-MS. Journal of Agriculture and Food Chemistry, 56, 661-669.
9. Hagerman, A. E. (2011). The Tannin Handbook. In, vol. 2016).
10. Inoue, K. H., & Hagerman, A. E. (1988). Determination of gallotannin with rhodanine. Analytical Biochemistry, 169, 363-369.
11. Kaur, M., Velmurugan, B., Rajamanickam, S., Agarwal, R., & Agarwal, C. (2009). Gallic Acid, an Active Constituent of Grape Seed Extract, Exhibits Anti-proliferative, Pro-apoptotic and Anti-tumorigenic Effects Against Prostate Carcinoma Xenograft Growth in Nude Mice. Pharmaceutical Research, 26(9), 2133-2140.
12. Kawada, M., Ohno, Y., Ri, Y., Ikoma, T., Yuugetu, H., Asai, T., Watanabe, M., Yasuda, N., Akao, S., Takemura, G., Minatoguchi, S., Gotoh, K., Fujiwara, H., & Fukuda, K. (2001). Anti-tumor effect of gallic acid on LL-2 lung cancer cells transplanted in mice. Anti-Cancer Drugs, 12(10), 847-852.
13. Krook, M. A., & Hagerman, A. E. (2012). Stability of polyphenols epigallocatechin gallate and pentagalloyl glucose in a simulated digestive system. Food Research International, 49(1), 112-116.
14. Lu, M-J., Chu, S-C., Yan, L., & Chen, C. (2009). Effect of tannase treatment on protein-tannin aggregation and sensory attributes of green tea infusion. LVVT—Food Science and Technology, 42(1), 338-342.
15. Nakamura, Y., Tsuji, S., Tonogai, Y. (2003). Method for analysis of tannic acid and its metabolites in biological samples: application to tannic acid metabolism in the rat. Journal of Agriculture and Food Chemistry, 51, 331-339.
16. Newsome, A. G., Li, Y., & van Breemen, R. B. (2016). Improved Quantification of Free and Ester-Bound Gallic Acid in Foods and Beverages by UHPLC-MS/MS. Journal of Agricultural and Food Chemistry, 64(6), 1326-1334.
17. Niemetz, R., & Gross, G. G. (2005). Enzymology of gallotannin and ellagitannin biosynthesis. Phytochemistry, 66(17), 2001-2011.
18. Pimpao, R. C., Dew, T., Figueira, M. E., McDougall, G. J., Stewart, D., Ferreira, R. B., Santos, C. N., & Williamson, G. (2014). Urinary metabolite profiling identifies novel colonic metabolites and conjugates of phenolics in healthy volunteers. Molecular Nutrition and Food Research, 58, 1414-1425.
19. Rodriguez, H., Rivas, B. d. I., Gomez-Cordoves, C., & Munoz, R. (2008). Degradation of tannic acid by cell-free extracts of Lactobacillus plantarum. Food Chemistry, 107(2), 664-670.
20. Ren, B., Wu, M., Wang, Q., Peng, X., Wen, H., McKinstry, W. J., & Chen, Q. (2013). Crystal Structure of Tannase from Lactobacillus plantarum. Journal of Molecular Biology, 425(15), 2737-2751.
21. Scalbert, A., Morand, C., Manach, C., Remesy, C. (2002). Absorption and metabolism of polyphenols in the gut and impact on health. Biomedicine and Pharmacotherapy, 56(6), 276-282.
22. Serrano, J., Puupponen-Pimia, R., Dauer, A., Aura, A.-M., & Saura-Calixto, F. (2009). Tannins: Current knowledge of food sources, intake, bioavailability and biological effects. Molecular Nutrition & Food Research, 53(S2), S310-S329.
23. Shahrzad, S., Aoyagi, K., Winter, A., Koyama, A., & Bitsch, I. (2001). Pharmacokinetics of gallic acid and its relative bioavailability from tea in healthy humans. The Journal of Nutrition, 131(4), 1207-1210.
24. Singleton, V. L., & Rossi, J. A. (1965). Colorimetry of Total Phenolics with Phosphomolybdic-Phosphotungstic Acid Reagents. American Journal of Enology and Viticulture, 16(3), 144-158.
25. Van der Pijl, P. C., Foltz, M., Glube, N., D., Peters, S., & Duchateau, G. S. M. J. E. (2015). Pharmacokinetics of black tea-derived phenolic acids in plasma. Journal of Functional Foods, 17, 667-675.
26. Wang, X., Ouyang, Y., Liu, J., Zhu, M., Zhao, G., Bao, W., & Hu, F. B. (2014). Fruit and vegetable consumption and mortality from all causes, cardiovascular disease, and cancer: systematic review and dose-response meta-analysis of prospective cohort studies (Vol. 349).
27. Wu, M., Wang, Q., McKinstry, W. J., & Ren, B. (2015). Characterization of a tannin acyl hydrolase from Streptomyces sviceus with substrate preference for digalloyl ester bonds. Applied Microbiology and Biotechnology, 99(6), 2663-2672.
28. Yao, J., Guo, G. S., Ren, G. H., & Liu, Y. H. (2014). Production, characterization and applications of tannase. Journal of Molecular Catalysis B: Enzymatic, 101, 137-147.

### Example 2: Evaluation of the Hydrolysis of Hydrolyzable Tannins from Mango (Gallotannins), Raspberry (Ellagitannins), Blackberry (Ellagitannins) and Guava (Gallo- and Ellagitannins) by Tannase Addition

Fruits, vegetables, and botanical consumption is linked to a reduction in chronic diseases. Some of the polyphenolics such as tannins in these plants have capacity to alter or delay onset of these diseases. Tannins are polyphenolics that are secondary metabolites in plants (Hager et al. 2008) with no known principle function in plants metabolism, biosynthesis, or biodegradation but they have the ability to afford protection against herbivores or against pathogenic attack (Hangerman 2002). Tannins have a relatively high molecular weight compared to most polyphenolics, and have hydroxyls groups which are linked to phenols. The structure may induce interactions between tannins and proteins, minerals, or other macromolecules (Vazquez-Flores et al. 2012). Tannins in foods may also result in bitterness and/or astringency (Olivas-Aguire et al. 2014). Tannins are broadly classified into two distinct groups in condensed and hydrolyzable forms. Condensed tannins or proanthocyanins are flavon-3-ol polymers in linear or branched configurations. Hyrdrolyable tannins further divided into gallotannins and ellagitannins (Hangerman 2002). Gallotannins are polymers of gallic acid esterified to a glucose core (Oliva-Aguirre et al. 2014). Ellagitannins are polymers of ellagic acid in the form of hexahydroxydiphenic acid esterified by two hydroxyl groups to a glucose core. Ellagitannins are commonly found in pomegranates, muscadine grapes, pecans, and walnuts along with various small fruits such as strawberries, raspberries, blackberries, and guava. Ellagic acid has been shown to exhibit antioxidant, anti-mutagenic, and anti-microbial activities. Tannin hydrolysis with tannase can reduce bitterness and astringency in juices and prevent sediment formation (Srivastava, Kar 2009).

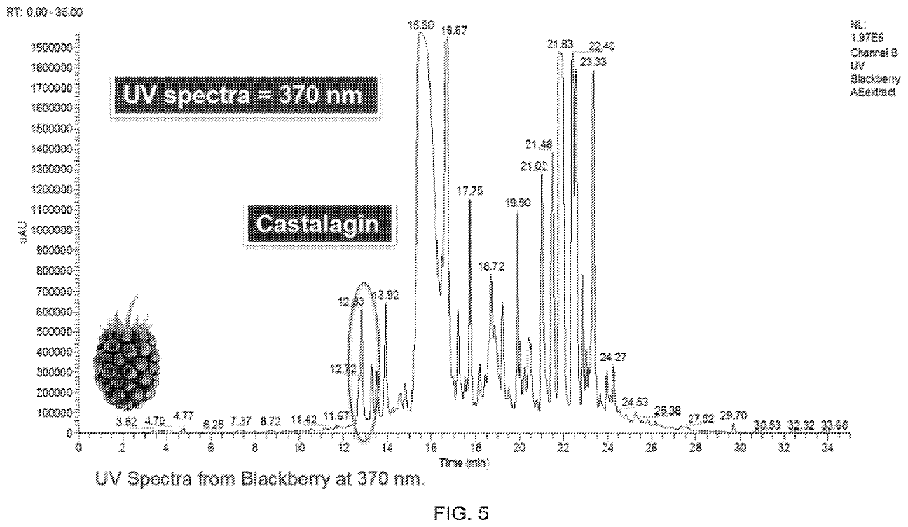

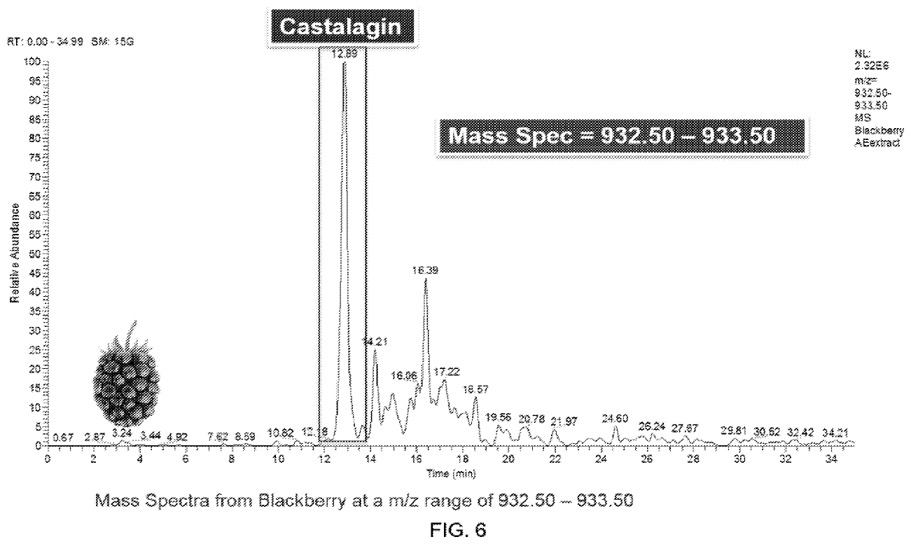

FIG. 5 shows an HPLC chromatogram of blackberry polyphenolics before tannase addition (20 U/mL) showing a diversity of polyphenolics and hydrolyzable tannins. Castalagin is an oligomeric ellagitannin naturally present in the fruit. FIG. 6 shows an HPLC chromatogram of blackberry polyphenolics after tannase addition (20 U/mL) showing a decrease in hydrolyzable tannins and an increase in castalagin, an oligomeric ellagitannin naturally present in the fruit.

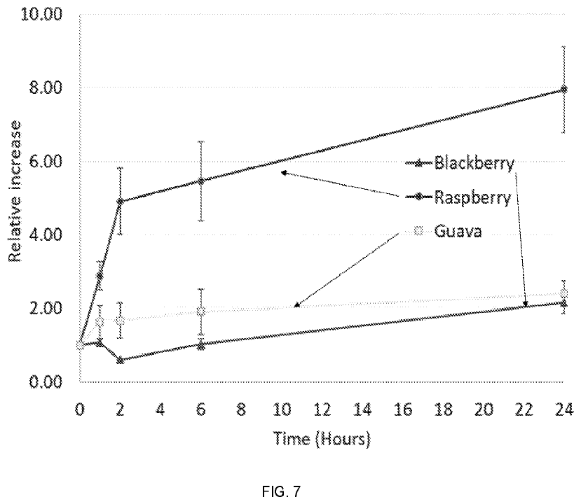

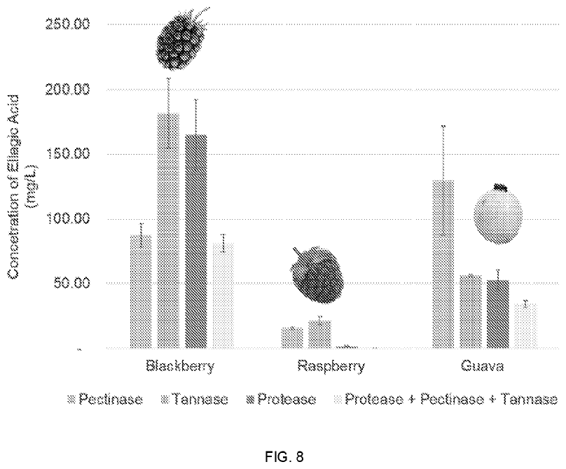

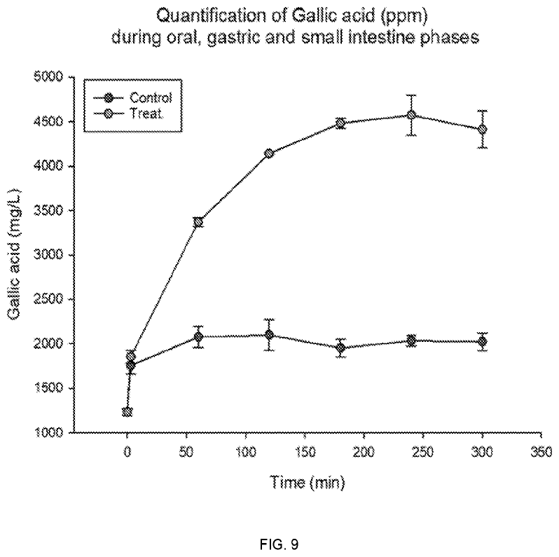

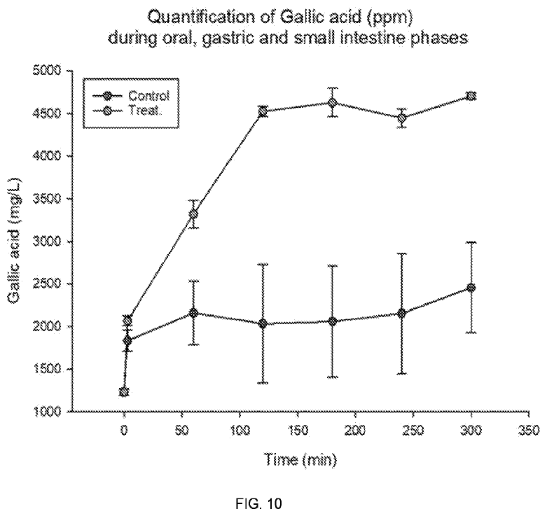

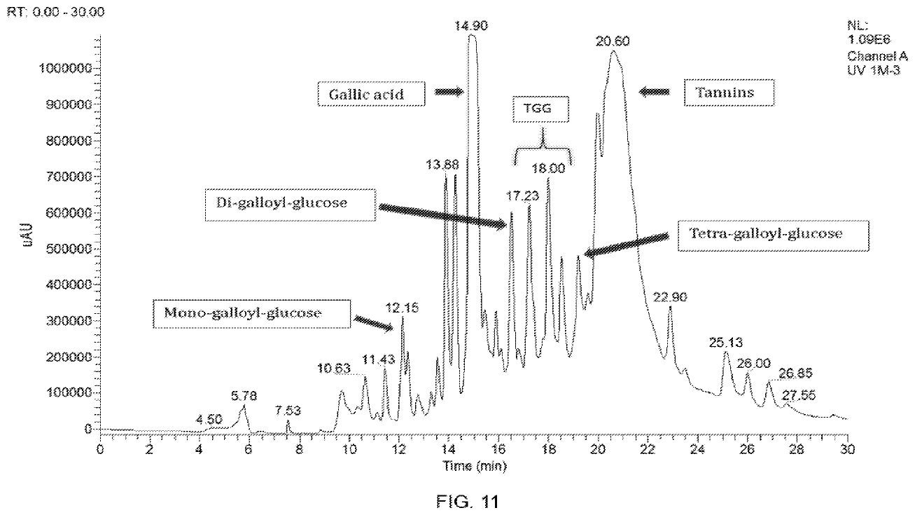

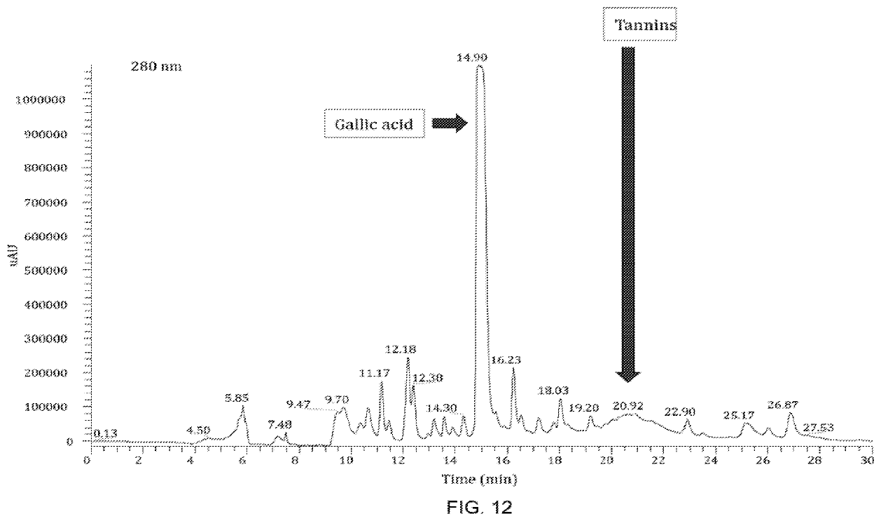

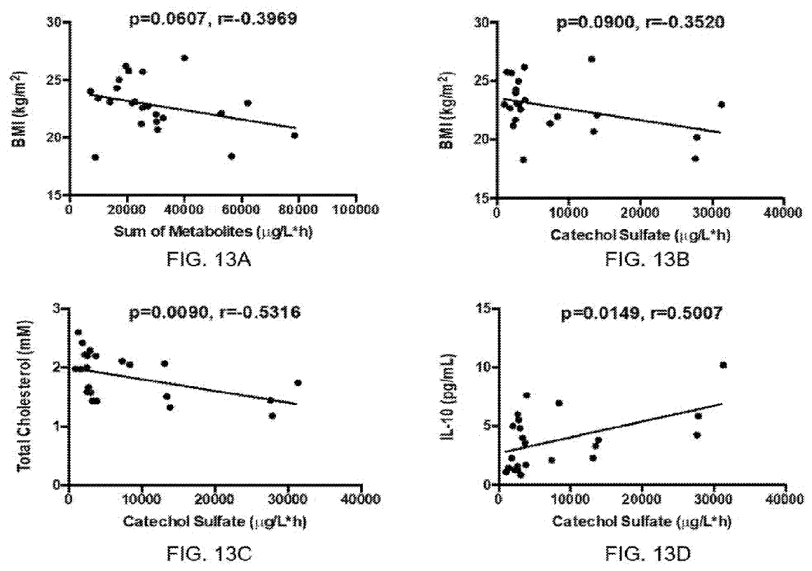

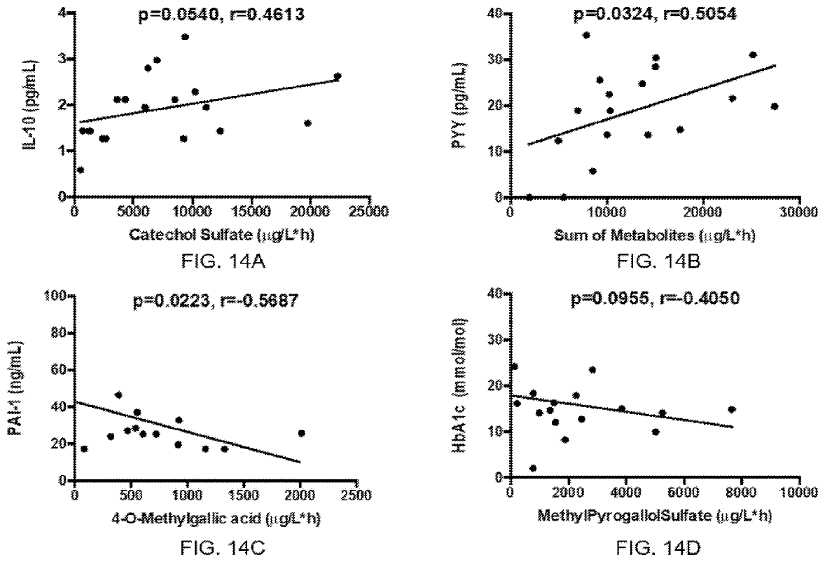

FIG. 7 shows the relative fold-increase in free ellagic acid for different fruits over 24 hrs in the presence of tannase (20 U/mL). The production of monometic ellagic acid is less pronounced than the production of oligomers from larger ellgitannin polymers. FIG. 8 is a bar graph of the hydrolysis of ellagitannins in different fruits over 4 hrs in the presence of tannase (20 U/mL) showing the concentration of free ellagic acid (mg/L) in the presence of processing aids (pecinase and protease) in effort to aid in the hydrolysis of ellagitannins.

### References for Example 2

1. Aguilar, Cristobal N.; Rodriguez, Raul; Gutierrez-Sanchez, Gerardo; Augur, Christopher; Favela-Torres, Ernesto; Prado-Barragan, Lilia A. et al. (2007): Microbial tannases: advances and perspectives. In Applied microbiology and biotechnology 76 (1), pp. 47-59. DOI: 10.1007/s00253-007-1000-2.
2. DuBois, Michel.; Gilles, K. A.; Hamilton, J. K.; Rebers, P. A.; Smith, Fred. (1956): Colorimetric Method for Determination of Sugars and Related Substances. In Anal. Chem. 28 (3), pp. 350-356. DOI: 10.1021/ac60111a017.
3. Gomori, G. (2010): Preparation of Buffers for Use in Enzyme Studies. In Roger L. Lundblad, F. Macdonald (Eds.): Handbook of biochemistry and molecular biology. 4th ed. Boca Raton, Fla.: CRC; London: Taylor & Francis [distributor], pp. 721-724.
4. Hager, Tiffany J.; Howard, Luke R.; Liyanage, Rohana; Lay, Jackson O.; Prior, Ronald L. (2008): Ellagitannin composition of blackberry as determined by HPLC-ESI-MS and MALDI-TOF-MS. In Journal of agricultural and food chemistry 56 (3), pp. 661-669. DOI: 10.1021/07199011
5. Hagerman, A. E. (2002): The Tannin Handbook, Biological Activity of Tannins. Miami University: Oxford, Ohio, USA. Available online at http://www.users.miamioh.edu/hagermae/.
6. Karamac, Magdalena; Kosinska, Agnieszka; Rybarczyk, Anna; Amarowicz, Ryszard (2007): Extranction and chomatographic separation of tannin fractions from tannin-rich plant material. In Polish Journal of Food and Nutrition Sciences 57 (4), pp. 471-474. Available online at http://journal.pan.olsztyn.pl/pdfy/2007/4/12_rozdzial.pdf, checked on Feb. 15, 2017.
7. Khanbabaee, K.; Ree, T. (2001): Tannins: Classification and Definition. In Nat. Prod. Rep. 18 (6), pp. 641-649. DOI: 10.1039/b1010611.
8. Komorsky, Sebojka; Novak, Ivana (2011): Determination of Ellagic Acid in Strawberries, Raspberries and Blackberries by Square-Wave Voltammetry. In International Journal of Electrochemical Science 6, pp. 4638-4647. Available online at http://www.electrochemsci.org/papers/vol6/6104638.pdf, checked on Feb. 7, 2017.
9. Lowry, Oliver H.; Rosebrough, Nira J.; Farr, A. Lewis; Randall, Rose J. (1951): PROTEIN MEASUREMENT WITH THE FOLIN PHENOL REAGENT. In J. Biol. Chem. 193 (1), pp. 265-275. Available online at http://www.jbc.org/content/193/1/265.full.pdf.
10. Nayeem, Naira; SMB, Asdaq (2016): Gallic Acid. A Promising Lead Molecule for Drug Development. In J App Pharm 08 (02). DOI: 10.4172/1920-4159.1000213.
11. Olivas-Aguirre, F.; Wall-Medrano, A.; Gonzalez-Aguilar, G.; Lopez-Diaz, Jose Alberto; Alvarez-Parrilla, Emilio; La Rosa, Laura A. de et al. (2014): Taninos hidrolizables; bioquimica, aspectos nutricionales y analiticos y efectos en la salud. In Nutricion hospitalaria 31 (1), pp. 55-66. DOI: 10.3305/nh.2015.31.1.7699.
12. Prinz, J. F.; Lucas, P. W. (2000): Saliva tannin interactions. In Journal of Oral Rehabilitation 27 (11), pp. 991-994. DOI: 10.1111/j.1365-2842.2000.00578.x.
13. Rout, S.; Banerjee, R. (2006): Production of tannase under mSSF and its application in fruit juice debittering. In CSIR 5 (3), pp. 346-350. Available online at http://hdl.handle.net/123456789/5594, checked on Feb. 28, 2017.
14. Sepulveda, Leonardo; Ascasio, Alberto; Rodriguez-Herrera, Raul; Aguilera-Carbo, Antonio; Aguilar Cristobal (2011): Ellagic acid: Biological properties and biotechnological development for production processes. In African Journal of Biotechnology 10 (22), pp. 4518-4523. Available online at http://www.academicjournals.org/AJB, checked on Feb. 7, 2017.
15. Srivastava, Anita; Kar, Rita (2009): Characterization And Application Of Tannase Produced By Aspergillus Niger ITCC 6514.07 On Pomegranate Rind. In Brazilian journal of microbiology: [publication of the Brazilian Society for Microbiology] 40 (4), pp. 782-789. DOI: 10.1590/S1517-83822009000400008.
16. Vázquez-Flores, Alma A.; Alvarez-Parrilla, Emilio; Lopez-Diaz, Jose Alberto; Wall-Medrano, Abraham; La Rosa, Laura A. de (2012): Taninos hidrolizables y condesados: naturaleza quimica, ventajas y desventajas de su consumo VI (2), pp. 84-93. Available online at https://www.researchgate.net/profile/Emilio_Alvarez-Parrilla/publication/264237320_Taninos_hidrolizables_bioquimica_aspectos_nutriciona les_y_analiticos_y_efectos_en_la_salud/links153f6b9a60cf22be01c4516e6.pdf, checked on 1 de Febrero 2017.

### Example 3: Evaluation of Tannase Hydrolysis of Sumac (Rhus coriaria) During Oral, Gastric and Intestinal Phases

Sumac is a fruit that is commonly dried and used as a condiment in the Middle East for its acidifying flavor or brewed into a tea used as a substitute for lemonade. The fruit has a high natural acidity and astringent flavor for its polyphenolic content. The gallotannins present in sumac can be hydrolyzed using the enzyme tannin-acyl-hydrolase (E.C. 3.1.1.20), also known as tannase. Studies evaluated the effect of enzymatic hydrolysis using tannin-acyl-hydrolase during in vitro gastrointestinal digestion using gallic acid as the marker for hydrolysis efficiency. During simulated oral, gastric and intestinal phases, the enzymatic hydrolysis performed by tannase degraded tannins of high molecular weight converting them into gallic acid and gallotannins of lower molecular weight. The concentration of gallic acid during enzymatic hydrolysis increased 282% when tested by the rhodanine assay and 256% gallic acid when analyzed by HPLC-MS analysis, showing good agreement between analysis methods. According to HPLC-MS analysis gallic acid and multiple gallotannins ranging in size from 1 to 9 gallic acid moieties were detected.

Sumac (Rhus coriaria L), belonging to the Anacardiaceae family, is widely used in all Middle Eastern countries, as a very popular condiment in food acidification (Brookie et al. 2018). Several components of this fruit are related to anticancer, antibacterial (Abu et al. 2014), and antiviral activity (Giusti 2014). These bioactive compounds include hydrolyzable tannins, anthocyanins, malic acid, flavonoids, terpene derivatives, and vitamins (Kossah 2010, Abu Reidah et al. 2014). Sumac consumption has been associated with anticancer properties, due to its high polyphenol content (Majd et al. 2017). Polyphenolic compounds are secondary metabolites of plants, fulfilling main functions within the metabolism of them and protecting from attack against predators and allelopathic interactions (Ricco et al. 2015). Among the polyphenols are tannins, which are subdivided into two large groups: condensed and hydrolyzable tannins. The condensed tannins are those that after hydrolysis release a proanthocyanidin; and the hydrolyzable tannins that, after an acid, basic or enzymatic hydrolysis, release gallic acid or ellagic acid (Camas 2016). Among the hydrolyzable tannins are the gallotannins, which are composed of a central glucose unit to which gallic acid units are esterified (Jourdes et al. 2013) and these in turn are linked by depside bonds (Olivas et al. 2015). Therefore, the enzyme tannase has been used to accelerate the hydrolysis process of these compounds to obtain, in a shorter time, units of free gallic acid. The enzyme tannase (EC 3.1.1.20) has been used in the degradation of tannins, catalyzing the hydrolysis of ester and depside bonds, without breaking the bonds between carbons, acting only on hydrolyzed tannins (Rodriguez et al. 2010). In this way, gallotannins are hydrolyzed, releasing gallic acid and glucose (Beniwal et al. 2013). This enzyme has a wide application in the food industry, especially in clarification of beers and fruit juices, in coffee beverages, and for the prevention of astringent flavors in wines, juices and instant tea. It is also used in the leather industry and the nutritive increase in livestock feed (Baik et al. 2015; Brahmbhatt and Modi 2015). The smaller a polyphenolic compound is, the greater the likelihood that it will be metabolized and absorbed (Talcott and Talcott 2009). Therefore, by obtaining more free gallic acid, there will be a greater absorption by the body, mainly in the portion of the small intestine. Gallic acid is an organic molecule that its molecular weight is 170.12 g/mol, which due to its biological activity (antioxidant capacity) is used in the pharmaceutical industry (PubChem 2004). This is because its hydroxyl groups donate electrons to neutralize free radicals, converting stable oxygen molecules; stopping the chain reactions caused by oxidation (Yilmazer 2018). There is an interest in this compound, due to its antioxidant properties and its possible beneficial implications for human health.

Crushed sumac was purchased from Cerez Pazari (Istanbul-Turkey). 200 g sumac and distilled water were added into a beaker and heated for 20 minutes at 35° C. Then it was filtered with mesh and filter paper using a vacuum pump. A final solution of 600 mL was obtained. Finally, the solution was stored at −20° C., prior to use in experiments

The enzyme tannin acyl hydrolase, EC 3.1.1.20 of bacterial origin of Aspergillus oryzae was used. 0.5 M citrate buffer was prepared at 5.5 pH, at a concentration of (1% tannase at −5,000 U/g). This solution was made immediately prior to its use. This solution was combined with a sumac dilution (33.333 g/L, 3.3%) and was incubated at 37° C. for 1 and 3 minutes during the oral phase (I), 60 minutes during gastric phase (II) and 60, 120, 180, 240 min during intestine phase (III). The enzymatic reaction was stopped by acidifying the samples (I and III) with methanol and placing them in boiling water for 30 seconds. This solution was analyzed by rhodanine assay and mass spectroscopy liquid chromatography (LC-MS).

In vitro digestion procedures were carried out based on methods reported by (Pinazo 2015) with some modifications. Solution-A, 210 mL (sumac 30×) was prepared and the pH was raised to 6.8 with the addition of 520 μL (6M NaOH). Three samples were taken to quantify the amount of gallic acid present before incubation with the enzyme tannase.
Oral phase. Solution-B (pH 6.7) was prepared, 30 mL of the solution-A was added 3 mL human saliva and incubated at 37° C. for 1 and 3 minutes with 100 μL (1% tannase). Three samples were taken, acidified and placed in boiling water for 30 seconds.
Gastric phase. Solution-C (pH 2.62) was prepared, 24 mL of solution-B was added 7 mL of gastric juice and incubated at 37° C. for 60 minutes. Three samples were taken from the continuous flow and placed in boiling water for 30 seconds.
Small intestine phase. Solution-D was prepared (pH 6.52), 29 mL of solution-C was added 12 mL (bile 12 mg/mL+pancreatin 2 mg/mL) dissolved in NaHCO₃ (0.1M). It was incubated al 37° C. for 60, 120, 180 y 240 minutes, respectively. Finally, three samples were taken, acidified and placed in boiling water for 30 seconds.

For the study a completely randomized design (CRD) was used, quantifying gallic acid and gallotannins, before and after enzymatic hydrolysis with separation of TUKEY stockings. The data was analyzed using the “Statistical Analysis System” (SAS version 9.4®).

The tannase enzyme during the oral phase degraded certain tannins from sumac in smaller molecules such as gallotannins and gallic acid. The content of gallic acid at minute one increased 50.46±2.50%, and at minute three, it increased 67.83±0.28% gallic acid, respectively (Table 2). However, there were no significant differences (P≤0.05) between treatments (with tannase) and controls (no tannase); due to incubation time, polyphenol-protein interaction and pH. The incubation times during this phase are relatively short (1 and 3 minutes). The tannase enzyme is incapable of breaking all the linkages between esters and m-depside bonds in such a short time period (Rodriguez et al. 2010; Chavez et al. 2016). So the enzymatic effectiveness at this concentration and activity was affected by incubation time (Barcena et al. 2013). Another factor to consider is the polyphenol-protein interaction. During digestion models is generally use bovine serum albumin (BSA) with α-amylase solution, however; during this study human saliva was used. Proline-rich salivary proteins (Prinz and Lucas 2000; Soares et al. 2018) and BSA (Ramirez 2012) have great affinity for binding tannins. Tannin-protein interaction is due to the union of hydrogen bonds through hydroxyl groups of phenolic compounds and carboxyl groups of protein bonds (Ramirez 2012). The pH is also considered a very relevant factor in the hydrolysis of tannins since it is related to the polyphenol-protein interaction and therefore in the enzymatic action. The tannases investigated by the Universidad Autonoma de Coahuila had a maximum activity between pH 4.3-6.5 using tannic acid as a substrate (Rodriguez et al. 2010).

In the gastric phase, incubated for 1 hr at pH 2.62±0.01 tannase continued to degrade the tannins of sumac into smaller molecules. The content of gallic acid at minute 61 increased 174.07±6.33% and at minute 63 it increased 169.46±9.74% gallic acid, respectively (Table 2).

In the intestinal phase, the pH was adjusted to 6.52±0.06 by the addition of sodium carbonate along with pancreatin and bile. During this phase the samples were evaluated during four hours and evaluations taken every hour. The intestinal phase produced the highest amount of free gallic acid, increasing 271 and 282% while the increase in gallic acid in the controls was 70% and 101% (Table 2). There are significant differences (10.05) between treatment, control, and blank (no tannase, no incubation time).

Several compounds of sumac extracts were specifically identified and characterized by HPLC-ESI-MS in negative mode. Free gallic acid was identified based on its molecular weight, fragmentation pattern and spectral pattern. In which an ion of m/z 205 was the most abundant, followed by an ion of m/z 169. That difference of 36 amu can be attributed to the link of two water molecules. In addition, MS2 showed a fragmentation pattern of 169 m/z and in some cases 125 m/z (Table 2). Mono-galloyl-glucose showed a predominant ion m/z 331 and an MS2 of 169 m/z, due to the loss of one molecule of glucose (180 g/mol) minus one molecule of water (18 g/mol) (Talcott and Talcott 2009). For higher molecular weight compounds such as penta-galloyl glucose, a similar ion of m/z 939 was found by further fragmentation to produce m/z 787 ions that corresponded to tetra-galloyl glucose. These fragments, created by inducing collisions with the parent compound, were in some way analogous to the effects of tannase hydrolytic enzymes, resulting in an indicator of the strength or weakness of chemical bonds present in gallotannins (Talcott and Talcott 2009). A second mass analysis was carried out, MS2, with the aim of knowing more specifically about the precursor ion (Ayala 2004), being more accurate in its characterization. Tannase generated numerous lower molecular weight gallotannin oligomers.

TABLE 2
Quantification of free gallic acid (mg/L) before and after enzymatic hydrolysis by rhodanine assay.

| | Blank¹ | Oral | Gastric | | | Intestinal (120-300) | |
|---|---|---|---|---|---|---|---|
| | Min. | | | | | | |
| Treat.² | 0³ | 1 & 3 | 60 | 120 | 180 | 240 | 300 |
| Control-1 min | 1,231 ± 42⁴ | 1,755 ± 119 | 2,075 ± 140 | 2,098 ± 214 | 1950, ± 121 | 2,034 ± 73 | 2,021 ± 122 |
| Control-3 min | 1,231 ± 42 | 1,834 ± 148 | 2,158 ± 455 | 2,034 ± 856 | 2,058 ± 803 | 2,153 ± 868 | 2,456 ± 650 |
| Tannase-1 min | 1,231 ± 42 | 1,853 ± 85 | 3,372 ± 60 | 4,139 ± 16 | 4,484 ± 66 | 4,575 ± 279 | 4,413 ± 259 |
| Tannase-3 min | 1,231 ± 42 | 2,066 ± 68 | 3,320 ± 198 | 4,528 ± 77 | 4,631 ± 204 | 4,448 ± 130 | 4,708 ± 47 |

¹Baseline (no tannase, no incubation time).
²Treatments (No tannase and with tannase for 1 or 3 minutes).
³Incubation time expressed in minutes.
⁴Mean and standard deviation.

The catalytic action of the enzyme tannase was successful, breaking down a majority of the tannins and increased the concentration of gallic acid by 248%. Some auto-hydrolysis also occurred in the control due to the non-enzymatic breakdown of the tannins at neutral pH. It is hypothesized that smaller polyphenolic compounds have a greater probability of absorption by the host before further oxidative or polymerization can occur in the gut. Mono-galloyl-glucose was also quantified after tannase treatment with its highest concentration in the gastric phase at 280 mg/L. This increase is because enzyme has as mechanism of action to release the ester bonds and bonds between gallic acids and glucose present in gallotannins (Kumar et al. 2006), but the last ester bond to the glucose may be more difficult to hydrolyze due to stearic hindrance at the active site of the enzyme

### References for Example 3

1. Abu Reidah I M, Ali Shtayeh M S, Jamous R M, Arráez Roman D, Segura Carretero A. 2014. HPLC-DAD-ESI-MS/MS screening of bioactive components from Rhus coriaria L. (Sumac) fruits. Elsevier.
2. Ayala C. 2004. Secuenciacion de proteinas por espectrometria de masas. Cuernavaca: UNAM.
3. BaikJH, Shin K-S, Park Y, Yu K-W, Suh H J, Choi H-S. 2015. Biotransformation of catechin and extraction of active polysaccharide from green tea leaves via simultaneous treatment with tannase and pectinase. J Sci Food Agric. 95(11):2337-2344. eng. doi:10.1002/jsfa.6955.
4. Bárcena J, Garcia C, Padilla C, Martinez E, Diez J. 2013. Caracterización cinética de la fosfatasa alcalina. [sin lugar].
5. Beniwal V, Kumar A, Sharma J, Chhokar V. 2013. Recent advances in industrial application of tannases: A review. Recent Pat Biotechnol. 7(3):228-233. eng.
6. Bermudez S. 2007. Stability of polyphenols in chokeberry (Aronia melanocarpa) subjected to in vitro gastric and pancreatic digestion. Food Chemistry. 102(3):865-874. doi:10.1016/j.foodchem.2006.06.025.
7. Brahmbhatt D, Modi H A. 2015. Comparative Studies on Methods of Tannas. International Journal for Research in Applied Science & Engineering Technology (IJRASET). 3.
8. Brookie K L, Best G I, Conner T S. 2018. Intake of Raw Fruits and Vegetables Is Associated With Better Mental Health Than Intake of Processed Fruits and Vegetables. Front Psychol. 9. eng. doi:10.3389/fpsyg.2018.00487.
9. Camas A. 2016. Utilización del método rodanina para determinar hidrólisis enzimática de compuestos del mango (Mangifera indica). [sin lugar]: Escuela Agricola Panamericana, Zamorano.
10. Chávez M, Buenrostro-Figueroa J, Rodriguez D, Zárate P, Rodriguez R, Rodriguez-Jasso M, Ruiz H, Aguilar C. 2016. Tannases. [sin lugar]: Elsevier. 471 p. p. 471-489.23 de sep. de 2016; [actualizado el 23 de sep. de 2016].
11. Giusti G. 2014. Rhus coriaria L. (Sommacco siciliano): studio della composizione dell'olio essenziale e dei volatili di piante spontanee raccolte in Sicilia. Stato dell'arte sulla composizione fitochimica, attivita biologica ed usi tradizionali della pianta. [sin lugar]: Universitá Di Pisa.
12. Hagerman A E, editor. 2002. Tannin chemistry. [sin lugar]: [sin editorial].
13. Jourdes M, Pouységu L, Deffieux D, Teissedre P-L, Quideau S. 2013. Hydrolyzable Tannins: Gallotannins and Ellagitannins. En: Ramawat K G, Merillon J M, editores. Natural products: Phytochemistry, botany and metabolism of alkaloids, phenolics and terpenes. Heidelberg, N.Y.: Springer. p. 1975-2010 (Springer reference).
14. Lassen A, Thorsen A V, Trolle E, Elsig M, Ovesen L. 2004. Successful strategies to increase the consumption of fruits and vegetables: Results from the Danish ‘6 a day’ Work-site Canteen Model Study. Public Health Nutrition. 7(2):263-270.
15. Majd N S, Coe S, Thondre S, Lightowler H. 2017. Determination of the antioxidant activity and polyphenol content of different types of <span class=“italic”>Rhus coriaria Linn</span>(sumac) from different regions. Proceedings of the Nutrition Society. 76(OCE4).
16. Mosele J, Macia A, Romero M, Motilva M J. 2016. Stability and metabolism of Arbutus unedo bioactive compounds (phenolics and antioxidants) under in vitro digestion and colonic fermentation. Food Chemistry. 201:120-130.
17. Negrete M. 2015. Hidrolisis enzimatica e identificación de galotaninos en el extracto de mango (Mangifera indica) [Pregrado]. [sin lugar]: Escuela Agricola Panamericana, Zamorano.
18. Olivas F, Wall A, Gonzalez G, Lopez J, Alvarez E, De la Rosa L, Ramoz A. 2015. Taninos hidrolizables; bioquimica, aspectos nutricionales y analiticos y efectos en la salud. Red de Revistas Cientificas de America Latina, el Caribe, Espana_{y} Portugal. 31(1):55-66.
19. Organización Mundial de la Salud. 2004. Estrategia Mundial sobre Regimen Alimentario, Actividad Física y Salud. [sin lugar]: [sin editorial].
20. Pinazo A. 2015. Evaluación in vitro de los cambios experimentados por las propiedades antioxidantes del fruto, hojas y fibra de caqui durante la digestion gastrointestinal. [sin lugar]:
21. Prinz J F, Lucas P W. 2000. Saliva tannin interactions. Journal of Oral Rehabilitation. 27(11):991-994. en.
22. PubChem. 2004. Gallic Acid. USA: U.S. National Library of Medicine; [actualizado 2018].
23. Ramirez C J. 2012. Factores fisicos y quimicos que intervienen en la interacción tanino-proteina y su relación con la sensación de astringencia. [sin lugar]: Universidad de Chile. es.
24. Ricco R, Agudelo I, Wagner M. 2015. https://www.researchgate.net/publication/288180597_Metodos_empleados_en_el_analisis_de_los_Polifenoles en un laboratorio de baja complejidad. [sin lugar]: Universidadde Buenos Aires.
25. Rodriguez L, Valdivia-Urdiales B, Contreras-Esquivel J, Rodriguez-Herrera R, Aguilar C. 2010. Quimica y biotecnologia de la tanasa. Revista Cientifica de la Universidad Autónoma de Coahuila. 2(4).
26. Rodriguez L V, Valdivia Urdiales B, Contreras Esquivel J C, Rodriguez Herrera R, Aguilar C N. 2010. Quimica y biotecnologia de la tanasa. Revista Cientifica de la Universidad Autónoma de Coahuila. 2(4).
27. Soares S, Garcia I, Ferrer-Galego R, Bras N, Brandao E, Silva M, Teixeira N, Fonseca F, Sousa S, Ferreira-da-Silva F, et al. 2018. Study of human salivary proline-rich proteins interaction with food tannins. Food Chemistry. 243:175-185.
28. Talcott S, Talcott S. 2009. Mass spectroscopic and HPLC characterization of mango (Mangifera Indica L.): Phytochemical Attributes Contribute to the Health-Promoting Benefits of Mangos. [sin lugar]: Universidad de Texas A&M.
29. Tanash R, Abdel-Dayem S, NOUR A. 2012. Optimization the hydrolysis process of tannic acid for gallic acid production by tannase of Aspergillus awamori using response surface methodology. [sin lugar]: [sin editorial].
30. Yilmazer A. 2018. Cancer cell lines involving cancer stem cell populations respond to oxidative stress. Biotechnology Reports. 17:24-30.

### Example 4: Increased Systemic Exposure to Tannase and Gallic-Acid Decarboxylase-Generated Metabolites is Associated with Increased Anti-Inflammatory Efficacy

Scope: Mangoes are a rich source of gallotannin-derived polyphenols that may exert anti-inflammatory effects relevant to obesity-related chronic diseases. This randomized human clinical study investigated the influence of daily mango supplementation for 6 weeks on inflammation and metabolic functions in lean and obese individuals.

Methods and results: Lean (n=12, BMI 18-25 kg/m²) and obese (n=9, BMI>30 kg/m²) participants, aged 18-65 years received daily 400 g of mango pulp for 6 weeks. Inflammatory cytokines, metabolic hormones and lipid profiles were examined in plasma before and after 6 weeks. In lean participants, systolic blood pressure was lowered by 4 mm Hg after 6 weeks. In obese participants, HbA1c and PAI-1 were reduced by 18% and 20%, respectively. Obese participants showed decreased plasma concentrations (AUC₀₋₈ₕ) of IL-8 and MCP-1. Correlation analysis indicates that the beneficial effects of mango supplementation on pro-inflammatory cytokines, PAI-1 and HbA1c are associated with systemic exposure to polyphenolic metabolites.

Conclusions: A linear correlation between microbial GT metabolites and anti-inflammatory efficacy indicates that increased plasma levels of these metabolites decrease pro-inflammatory cytokines and regulate metabolic hormones in obese participants potentially in a concentration-dependent manner.

### Example 5: Gallotannins and Lactobacillus plantarum WCFS1 Reduce Inflammation and Insulin Resistance and Increase Thermogenesis in High-Fat Diet-Fed Gnotobiotic Mice

Scope: Intestinal microbial metabolites from gallotannins (GT), including gallic acid (GA) and pyrogallol (PG), may possess potential anti-obesogenic properties. Lactobacillus plantarum (L. plantarum) found in the intestinal microbiome encodes for enzymatic activities that metabolize GT into GA and PG. Anti-obesogenic activities of orally administered GT in the presence or absence of L. plantarum was examined in gnotobiotic mice fed a high-fat diet (HFD).

Materials and Methods

2.1. Extract Preparation and Characterization

Tannic acid and GA were purchased from Sigma-Aldrich (St. Louis, Mo.). Sephadex LH-20 (Sigma-Aldrich, St. Louis, Mo.) was used to isolate GT and remove residual GA [27]. A 250 mL column was filled to 25% capacity with Sephadex-LH-20 and the resin was rehydrated with 100% ethanol. 1% tannic acid in 0.1% formic acid was loaded onto the column, washed with 1 column volume of 100% ethanol, and eluted with acetone and 0.1% formic acid (80:20). Acetone was evaporated under reduced pressure at 45° C. The resulting concentration of the tannic acid isolate was 11423 mg L⁻¹ gallic acid equivalent (GAE). To confirm the purity of GT, the extract was analyzed using a Thermo-Finnigan Surveyor high-performance liquid chromatography-photodiode array detector (HPLC-PDA) in tandem with a LCQ Deca XP Max ion trap spectrometer with an ESI source as previously described [28]. GTs and their derivatives were characterized at 280 nm.

2.2. Enzymatic Activities of L. plantarum WCFS1

L. plantarum WCFS1 was purchased from American Type Culture Collection (ATCC, Rockville, Md.) and routinely grown in MRS broth (Difco, Detroit, Mich.) in an anaerobic environment at 37° C. For characterizing tannase and decarboxylase activities of L. plantarum, bacteria were grown on MRS broth until early exponential phase (optical density at 600 nm [OD600]=0.3). Cultures were added to a fresh modified medium (6 g (NH₄)₂SO₄, 0.4 g MgSO₄7H₂O, 7 g KH₂PO₄, 0.02 g FeSO₄7H₂O, 3 g Casamino acids (Sigma-Aldrich, St. Louis, Mo.) in 1 L of water, pH: 5.5) [29]) supplemented with 0.5 mM GT or 1.5 mM GA. Cultures were continued to grow to mid-exponential phase (OD600=0.6), centrifuged at 4° C. for 10 minutes at 3000 rpm, and washed twice with phosphate-buffered saline (PBS) (pH 5.8). Afterwards, cultures were re-suspended in PBS supplemented with 0.5 mM GT or 1.5 mM GA. To investigate tannase activity of L. plantarum, aliquots of sample were removed at 0, 1, 6, 12, 24 hours after the addition of GT. As for the decarboxylase activity, aliquots of sample were removed at 0, 15, 30, 60, and 120 minutes after the addition of GA. Acidified methanol was added to samples. Samples were filtered and analyzed by high-performance liquid chromatography-mass spectrometry (HPLC-MS) [30]

2.3. Animal Study Design

GF C57BL/6J mice were maintained under GF conditions in a room with a 12-hour light-dark cycle and routinely monitored for GF status by standard microbiological methodologies [31]. All procedures were performed inside a sterile, flexible-film isolator, unless microorganisms were intentionally introduced. GF mice were randomly divided into three groups: non-colonized GF mice received a vehicle solution (GF-Con) or GT (GF-GT), and one group was colonized with L. plantarum and received GT (Lp-GT), for five weeks. After 1 week of acclimation with regular diet (D12450J, Research Diets, New Brunswick, N.J.), mice were orally gavaged with 100 μL of either saline or L. plantarum (10⁸ CFU/100 μL) for three consecutive days (Week 0-1: Day 1 to Day 3). Colonization of gnotobiotic mice was monitored by fecal 16S ribosomal RNA (rRNA) analysis [32]. After L. plantarum colonization, mice were gavaged with GT (1.6 mg/mouse/day) on alternating days for 5 weeks. After adaptation of animals to intestinal colonization and administration of GT, a HFD containing 60% kcal fat (D12492-1.5V, Research Diets, New Brunswick, N.J.) was given for the last 4 weeks of this study. Mice were sacrificed, and blood, tissues and feces were collected, weighed, and stored at −80° C. until further analysis. The animal use protocol was approved by the Institutional Animal Care and Use Committee of Texas A&M University (IACUC#2016-0087).

2.4. Inflammatory Cytokines and Metabolic Hormones

Diet-induced obesity is associated with low-grade systemic inflammation and insulin resistance [33]. In this study, the plasma levels of inflammatory cytokines, including tumor necrosis factor α (TNF-α) and MCP-1; and metabolic hormones, including insulin and leptin were determined by multiplex bead assay (Millipore, Billerica, Mass.). These experiments were performed on a Luminex L200 machine (Luminex, Austin, Tex.) and data were analyzed by Luminex ×PONENT software version 3.1. Differences of TNF-α, MCP-1, and leptin were compared between Week 2 and Week 6. Fasting blood glucose levels were determined using a Cayman glucose colorimetric assay kit (Cayman Chemical Company, Ann Arbor, Mich.). Homeostasis Model Assessment of Insulin Resistance (HOMA-IR) was calculated based on the following formula: fasting plasma glucose (mmol/L)×fasting plasma insulin (pU/mL)/99.95 [34].

2.5. Quantitative RT-PCR

Total RNA was isolated from adipose tissues using the mirVana™ miRNA Isolation Kit (Applied Biosciences, Foster City, Calif.) according to the manufacturer's protocol. The concentration of the extracted RNA was determined using the NanoDrop ND-1000 spectrophotometer (NanoDrop Technologies, Wilmington, Del.). Briefly, 1000 ng of RNA was used to synthesize cDNA using a Reverse Transcription Kit (Invitrogen, Grand Island, N.Y.). SYBR Green PCR Master Mix (Applied biosystems, Foster City, Calif.) was used for the qPCR analysis on the CFX384 Touch Real-Time PCR Detection System (Bio-Rad, Hercules, Calif.). Gene expression levels of carnitine palmitoyltransferase I (CPT1), perilipin 1, hormone-sensitive lipase (HSL), transmembrane protein 26 (Tmem26), T-box transcription factor 1 (Tbx1), peroxisome proliferator-activated receptor γ coactivator 1α (PGC1α), cyclooxygenase 2 (Cox2), PR domain containing 16 (PRDM16), and cytochrome c oxidase 7a1 (Cox7a1) were analyzed by qPCR, and data were normalized to β-actin as an endogenous control [11].

2.6. Western Blotting

Adipose tissues were homogenized and lysed in T-PER tissue protein extraction reagent (Pierce, Rockford, Ill.) containing 1% Halt protease and phosphatase inhibitor cocktail (Thermo Scientific), and centrifuged at 12,000 g for 15 minutes at 4° C. The layer below the fat was collected and centrifuged again [35]. Protein was then quantified by the Bradford assay (Invitrogen, Carlsbad, Calif.), loaded and run on a 4-12% sodium dodecyl-polyacrylamide gel and transferred to a PVDF membrane using the iBlot Dry Blotting system (Invitrogen, Carlsbad, Calif.). The membrane was blocked in 5% non-fat milk solution for 1 hour and probed with primary antibodies against phosphorylated AMPKα1 (p-AMPKα1), total-AMPKα1 (t-AMPKα1), CCAAT/enhancer binding protein a (C/EBPα), peroxisome proliferator-activated receptor y (PPARγ), fatty acid synthase (FAS), Sirtuin1 (SIRT1), uncoupling protein 1 (UCP1), and β-actin (Cell Signaling Technology, Danvers, Mass.) [11]. The band intensity in Western blot was determined using ImageJ software (National Institutes of Health, Bethesda, Md., USA; http://rsb.info.nih.gov/ij/).

2.7. Histological Analyses

Adipose tissues were dehydrated, embedded in paraffin, and sectioned at 5 μm of thickness. Hematoxylin and eosin (H&E) staining was performed as previously described [36]. Images of each section from each mouse were obtained with a Zeiss Axioplan 2 microscope (Carl Zeiss, Thornwood, N.Y.) fitted with an Axiocamhigh resolution digital camera and Axiovision 4.1 software using the same settings.

2.8. Statistical Analyses

The data were analyzed using GraphPad Prism 6 (GraphPad Software, Lo Jolla, Calif.). Results are presented as means±standard error of the mean (SEM). In this study, outliers were identified in female and male mice before pooling the data using the ROUT method in GraphPad Prism 6. p values were calculated using one-way ANOVA if data were normally distributed or the Kruskal-Wallis test if data were not normally distributed. A p value less than or equal to 0.05 indicates statistical significance between groups and is marked with different letters above the data.

Results

3.1. Tannase and Decarboxylase Activities of L. plantarum

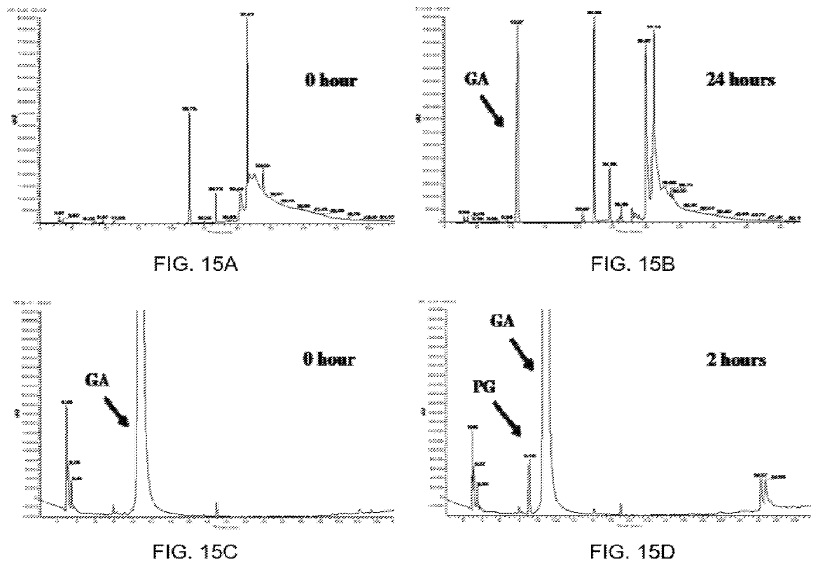

Activities of GT-metabolizing enzymes in L. plantarum cultures were assessed using HPLC-MS. GA, the product of microbial hydrolysis of GT by tannase produced by L. plantarum, was detected in L. plantarum cultures at 280 nm after 24 hours incubation with 0.5 mM GT (FIG. 15A, 15B). L. plantarum is known to produce gallate decarboxylase that catalyzes the decarboxylation of GA to produce PG [30]. In this study, PG was detected in L. plantarum cultures incubated with 1.5 mM GA for 2 hours at 280 nm (FIG. 15C, 15D).

3.2. Characterization of GT Extract

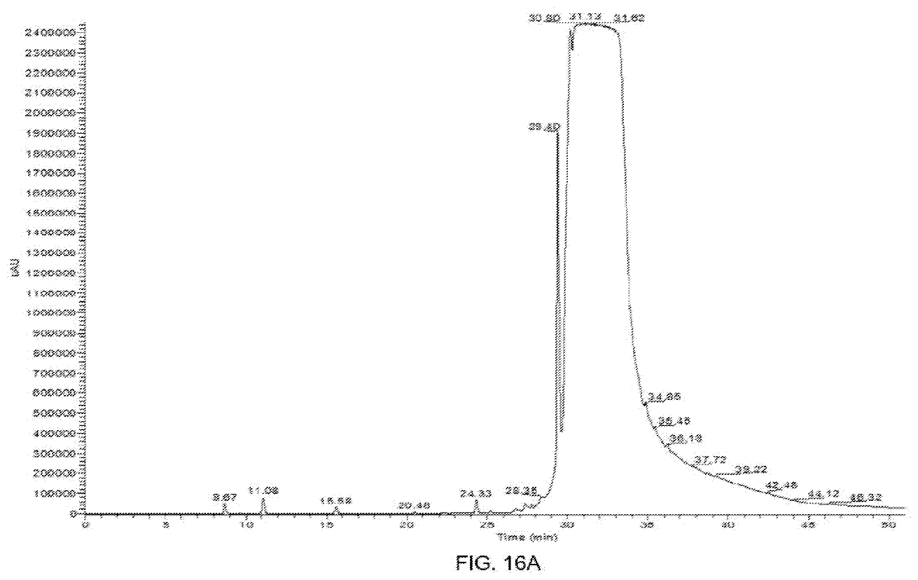

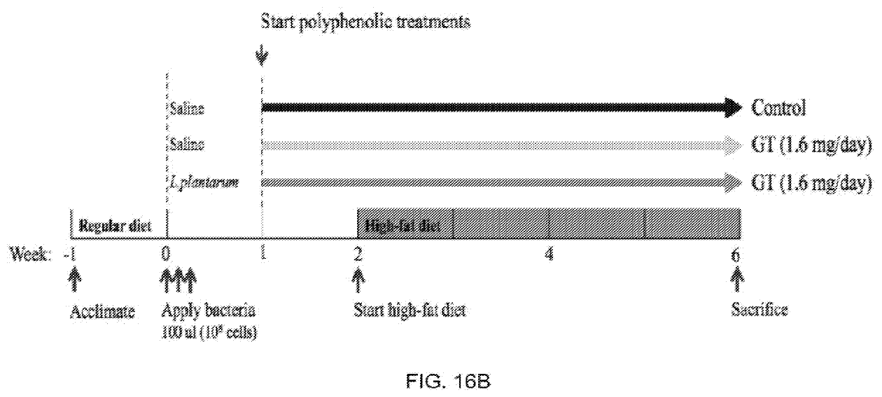

Using liquid chromatography-electrospray ionization-tandem mass spectrometry (LC-ESI-MS), GT with a degree of polymerization of 5 and greater were detected at 280 nm at a retention time of 25-55 minutes, and no GA or other small absorbable compounds were present in the GT extract (FIG. 16A).

3.3. L. plantarum Colonization Did not Affect Average Body Weight and Adiposity but Improved Metabolic Functions

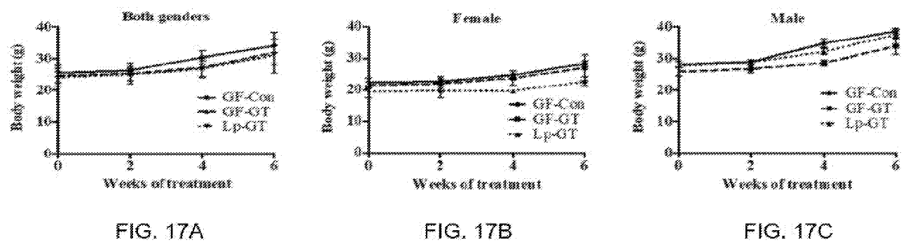

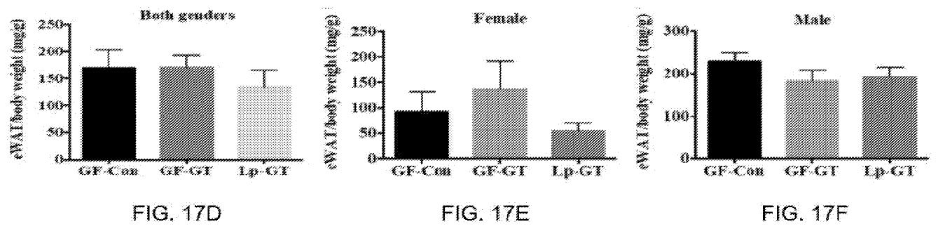

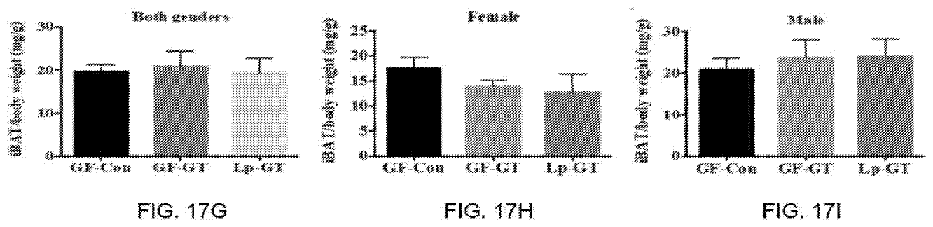

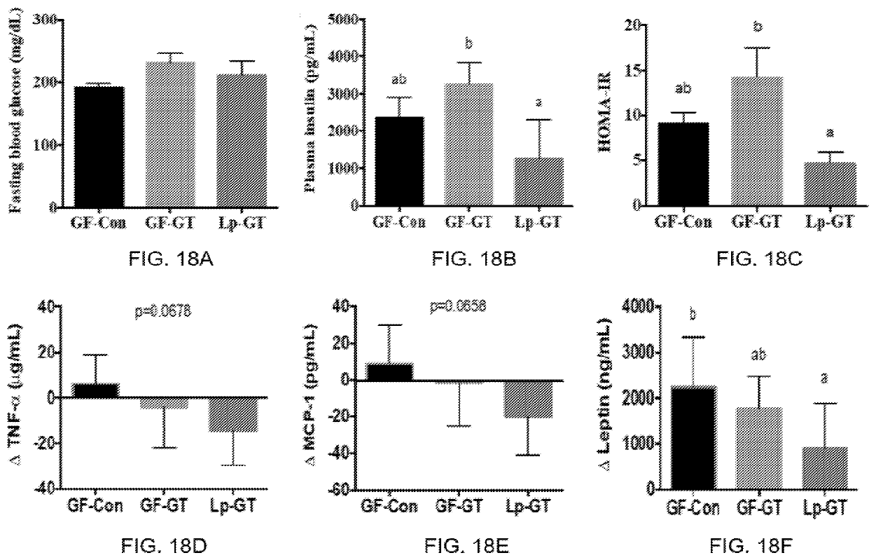

Overall, gnotobiotic mice in each study group (n=7, female=3, male=4, FIG. 16B) showed no significant difference in average body weight (FIG. 17A-17C) and fat mass including epididymal WAT (eWAT) (FIG. 17D-17F), and interscapular BAT (iBAT) (FIG. 17G-171) after 4 weeks of HFD feeding. Gender-specific physiological differences between female and male mice may impact the results of this study; therefore male and female mice were evaluated separately as well as in data pools within each treatment group. Fasting plasma glucose levels were similar in three groups (FIG. 18A). Mice colonized with L. plantarum had significantly lower level of insulin (p=0.0043) and HOMA-IR (p=0.0111) when compared to the GF group treated with GT (FIG. 18B, 18C). GF mice treated with GT experienced a non-significant decreased TNF-α, MCP-1, and leptin levels. Intestinal colonization with L. plantarum significantly alleviated the HFD-induced increases of TNF-α, MCP-1, and leptin by 337.63% (p=0.0183), 330.53% (p=0.0234), and 59.94% (p=0.0330) between Weeks 2 and 6, respectively (FIG. 18D-18F).

3.4. GT and GT with L. plantarum Colonization Modulated the Expressions of Molecules Involved in Lipid Metabolism and Reduced Lipid Size in eWAT

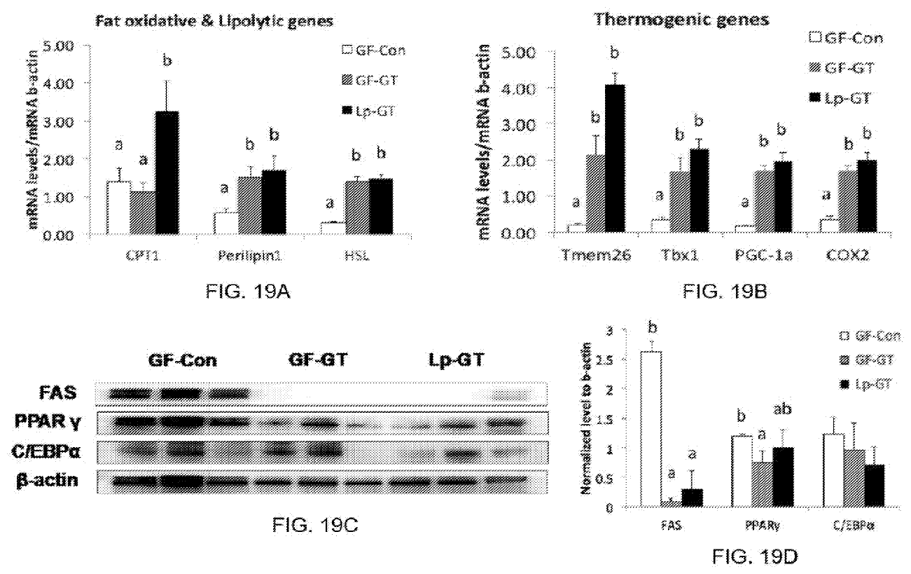

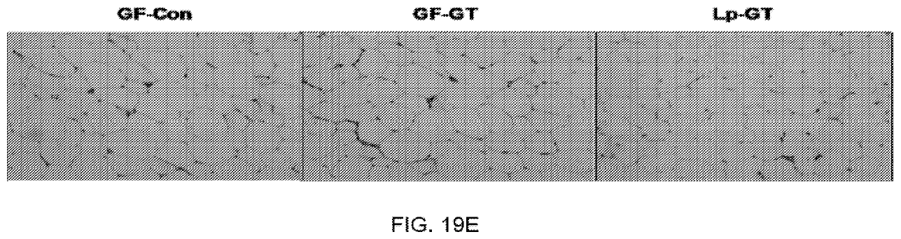

White adipose tissue has its classical role as a lipid storage organ, as well as other critical roles in endocrine function associated with a wide range of metabolic disorders [37]. In eWAT, both GF-GT and Lp-GT groups exhibited increased mRNA expressions of lipolytic (perilipin 1 and HSL) and thermogenic (Tmem26, Tbx1, PGC1α, Cox2) genes. In addition, Lp-GT group exhibited increased CPT1 mRNA expression compared to the GF groups (FIG. 19A, 19B). Lipid accumulation is highly regulated by key transcription factors (e.g., PPARγ and C/EBPα) and enzymes involved in fatty acid synthesis (e.g., FAS) [11, 38]. GT treatment down-regulated the protein expressions of FAS and PPARγ. Additionally, C/EBPa showed a trend towards decreased levels (FIG. 19C, 19D). Morphologically, Lp-GT group was characterized by more of the multi-locular lipid droplets in the eWAT than the control group as shown by H&E staining (FIG. 19E), and this suggests reduced lipid size in L. plantarum-colonized mice. GT treatment alone did not affect the size of lipid droplets (FIG. 19E).

3.5. GT and GT with L. plantarum Colonization Modulated Lipid Metabolism and Enhanced Thermogenesis in iBAT

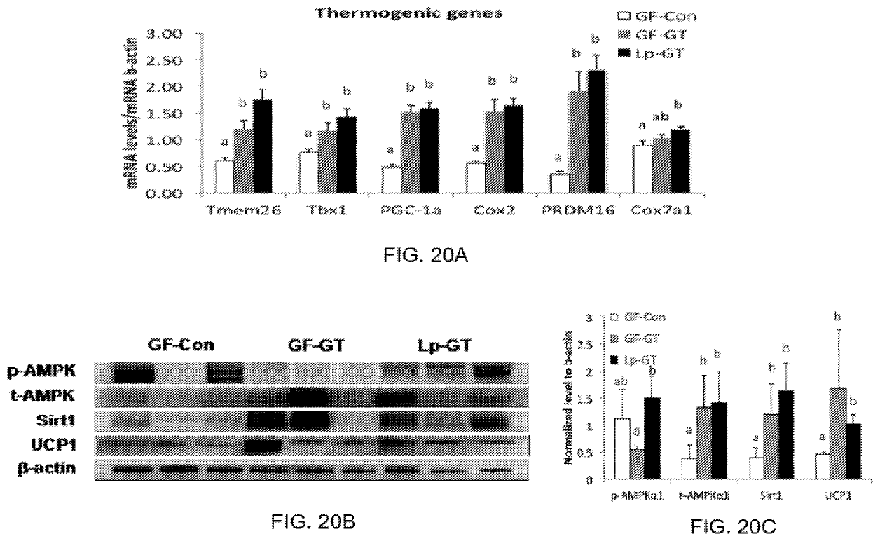

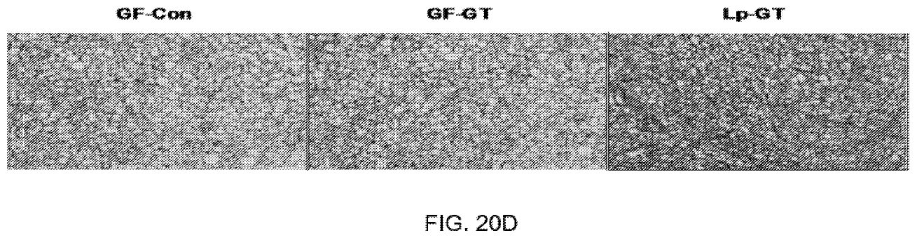

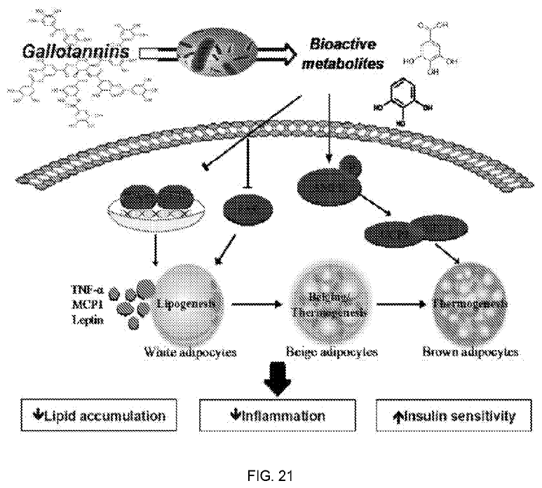

Brown adipose tissue is specialized in dissipating energy through thermogenesis and has been implied as relevant to the prevention and treatment of obesity [39]. In iBAT, the mRNA expressions of thermogenic genes (Tmem26, Tbx1, PGC1α, Cox2, PRDM16 and Cox7a1) were increased in both GF-GT and Lp-GT groups; Cox7a1 mRNA expression was further enhanced by L. plantarum colonization compared to the GT treatment alone, but not significantly so (FIG. 20A). The AMPK pathway plays a pivotal role in energy metabolism and is highly expressed in brown adipose tissue [40]. Previously, GT derivatives (e.g., PG) from mango polyphenolic extract have been shown to induce the browning of white adipocytes into beige adipocytes, which might be associated with the activation of the AMPK pathway [11]. Accumulating evidence suggests that some polyphenols (e.g., resveratrol and procyanidins) induce the formation of the brown-like adipocytes through the phosphorylation of AMPKα1 and enhancing the expressions of brown adipocyte markers such as UCP1, SIRT1, PGC1α, and PRDM16 [36, 41]. The protein expressions of UCP1, SIRT1, and t-AMPKα1 were up-regulated in the GT-treated groups while p-AM PKa1 were additionally up-regulated in the Lp-GT group (FIG. 20B, 20C), suggesting the activation of the AMPK pathway and enhanced thermogenesis in iBAT by L. plantarum colonization. The H&E staining further confirmed our hypothesis that GT in combination with L. plantarum induces thermogenesis and reduces lipid size in iBAT (FIG. 20D).

### 4. Discussion

Plant-based bioactive compounds from fruits and vegetables have been found to inhibit lipogenesis while promoting brown and beige adipocyte development and thermogenesis and are therefore considered novel nutritional intervention strategies in the prevention of obesity and its related chronic diseases. Mechanisms underlying the anti-obesogenic activities of some polyphenols, including epigallolcatechin gallate (EGCG) [42, 43], resveratrol [44], quercetin [45, 46], and curcumin [47-49] have been investigated in in vitro, in vivo, and human clinical studies. While anti-inflammatory and anti-cancer activities of polyphenols such as GTs and their derivatives (GA and PG) have been examined in breast cancer [50] and colitis [51, 52] models, investigation of these polyphenols in obesity seems to be limited. Mango polyphenolic extract (high in GT and GA) and a purified compound PG inhibit adipogenesis and reduce lipid accumulation and PG additionally promotes thermogenesis in 3T3-L1 adipocytes [11]. It has yet to be determined whether the beneficial effects are attributed to the parent compound GT or the production of microbial GT metabolites GA and PG by tannase and decarboxylase produced by gut microbiota (e.g. L. plantarum). Therefore, this study aimed to investigating whether the intestinal colonization with L. plantarum can improve the bioactivities of GT administered to GF mice.

In this study, GT non-significantly reduced HFD-induced inflammation at least in part through inhibiting fat synthesis in eWAT and promoting thermogenesis in iBAT. In eWAT, GT-treated mice exhibited lower expressions of lipid synthesis enzymes (FAS, PPARγ and C/EBPα), and higher expressions of molecules involved in lipolysis (perilipin1 and HSL) and thermogenesis (Tmem26, Tbx1, PGC-la, and Cox2) compared to GF mice treated with a vehicle solution. Similarly, in iBAT the expressions of thermogenic markers (SIRT1, UCP1, Tmem26, Tbx1, PGC-1α, Cox2, and PRDM16) were significantly higher in GT-treated groups. However, feeding GF mice with GT alone did not affect fasting blood glucose, insulin, and HOMA-IR levels compared to vehicle-treated mice. In this study, GT was administered in the form of tannic acid that contains GA oligomers of 5 and greater. These GA oligomers are not absorbable and are subject to hydrolysis, decarboxylation, and other reactions by intestinal microbial bacteria that yield absorbable GT metabolites [24, 25]. GT treatment alone without the addition of L. plantarum demonstrated beneficial effects on modulating inflammatory responses and adipose tissue functions. Previously, the polyphenols-lipid/protein binding activity was proposed as a possible mechanism of reduced obesity and inflammation after HFD feeding [53]. In this study, the binding of dietary polyphenols GT to lipids and proteins in the intestine may interfere with the bioactivity of enzymes involved in signaling transduction, leading to impaired macronutrient digestion, metabolism, and absorption [53]. These body weight- and fat-lowering effects might furthermore alleviate HFD-induced inflammation and adipose tissue dysfunction, which is in line with our findings.

In addition to the GT treatment, colonization with L. plantarum significantly improved biomarkers for inflammation and insulin resistance. HFD-induced insulin resistance and inflammation (TNF-α, MCP-1, and leptin) were lower in L. plantarum-colonized group compared to GF-GT group. No effect on average body weight, adiposity, and fasting blood glucose level was observed for either treatment groups possibly due to the short duration of the study. The expressions of CPT1 in eWAT, as well as p-AMPKα1 and Cox7a1 in iBAT were further increased after the colonization with L. plantarum. L. plantarum encodes for GT-metabolizing enzymes that yield absorbable bioactive metabolites, namely GA and PG in the gnotobiotic mouse model. Both eWAT and iBAT of L. plantarum-colonized mice were characterized by smaller, multi-locular lipid droplets. These results support the hypothesis that L. plantarum has the potential in reducing obesity-associated inflammation and insulin resistance, at least in part through GT-metabolizing activities that generate absorbable, bioactive microbial metabolites (FIG. 20).

Emerging evidence demonstrates the two-way relationship between dietary polyphenols and the composition of the intestinal microbiota where an increased intake of polyphenols may shape the composition of the intestinal microbiota by increasing species with the ability to metabolize polyphenols [54-57]. Many so-called probiotic species such as Streptococcus gallolyticus, Lonepinella koalarum, Bacillus licheniformis, and several Lactobacilli species fall into this category [58]. Daily consumption of GT-rich mango pulp for 6 weeks increases levels of tannase-producing bacteria (Lactoccoccus lactis) in healthy human subjects, and this increase was correlated with increased tannase enzyme activity in fecal samples. The production of a short-chain fatty acid, namely butyrate showed a trend towards increased levels after 6-week mango consumption [59]. This evidence suggests that the health-promoting effects of GT and L. plantarum may be at least in part based on prebiotic-probiotic interactions between GT and L. plantarum. Potentially, non-absorbable GTs mediate their anti-inflammatory and anti-obesogenic activities indirectly through increasing the abundance of L. plantarum. In support of this hypothesis, the supplementation (22 weeks) of green tea polyphenols combined with L. plantarum reduced body fat content and cholesterol accumulation, and additionally promoted the growth of Lactobacillus species in the intestine and attenuated HFD-induced inflammation in HFD-induced obese mice [60]. In addition, GT-induced increased abundance of L. plantarum may result in increased short-chain fatty acid production. In antibiotic-associated diarrhea patients, a significant increase was noted for the production of butyrate in fecal samples of patients receiving an L. plantarum-fermented fruit drink compared to patients receiving a placebo fruit drink [61]. Butyrate as a microbiota-induced fermentation product has shown anti-inflammatory and anti-obesogenic potential possibly due to its ability in enhancing intestinal barrier integrity and function [62].

In addition to enhancing probiotic activities, the production of bioactive GT metabolites via microbial degradation can be associated with decreased inflammation and risk of developing obesity-related metabolic disorders [13]. L. plantarum possesses tannase- and decarboxylase-producing activities in degrading large, unabsorbable GT into small, absorbable, bioactive compounds GA and PG that are easily distributed into tissues where they can act as anti-inflammatory and anti-obesogenic agents [11, 12, 19]. This may enhance the health-promoting effects derived from GT-rich food in reducing obesity and its related chronic diseases. Taken together, it remains to be investigated to what extent the beneficial effects of GT in combination with L. plantarum are attributed to the enhanced growth of GT-metabolizing bacteria or the increased systemic exposure to GT derivatives.

Findings in this study provide evidence for the beneficial role of probiotics in context with a polyphenol-rich diet. These findings need to be validated in future animal studies with larger animal numbers in each group and longer periods of time. Overall, it remains uncertain, to what extent the anti-obesogenic effects improving WAT and BAT functions are based on GT metabolites or the presence of the probiotic species L. plantarum. This study has the potential to link the biological activities of dietary polyphenols to gut microbial composition and provide novel insights into dietary recommendations that include probiotics into our diet to increase bioavailability and bioefficacy of dietary polyphenols. Future pharmacokinetic/pharmacodynamic analyses should characterize polyphenolic profiles in plasma and adipose tissue to understand the role of individual bioactive GT metabolites.

### 5. Conclusions

Overall, orally administered GT reduced HFD-induced inflammation and lipid accumulation in eWAT and promoted thermogenesis in iBAT. Colonization with L. plantarum further reduced adipose tissue expansion, inflammation, and insulin resistance. This study suggests that GT treatment exerts health benefits at least in part through the polyphenol-protein binding activity that lowers the macronutrient absorption and subsequently reduces inflammation and improves adipose tissue function. In addition, the potential role of prebiotic-probiotic interactions in the production of absorbable, bioactive microbial GT metabolites has been revealed. Enhanced bioavailability and bioefficacy of GT derivatives might be responsible for their anti-inflammatory and anti-obesogenic activities after the colonization with L. plantarum. Together, these findings have implications for a future human clinical trial in which subjects are supplemented with dietary GT with or without probiotics to investigate if the health-promoting effects of GT are attributed to GT itself or the production of microbial GT metabolites. This study has the potential in providing dietary guidelines and recommendations in terms of the bioefficacy of a polyphenol-rich diet in the presence of probiotics.

### References for Example 5

1. Surmi, B., Hasty, A., Macrophage infiltration into adipose tissue: initiation, propagation and remodeling. Future lipidology 2008, 3, 545-556.
2. McArdle, M. A., Finucane, O. M., Connaughton, R. M., McMorrow, A. M., Roche, H. M., Mechanisms of obesity-induced inflammation and insulin resistance: insights into the emerging role of nutritional strategies. Frontiers in endocrinology 2013, 4, 52.
3. Büther, M., Adipose tissue inflammation: a cause or consequence of obesity-related insulin resistance? Clinical science 2016, 130, 1603-1614.
4. Meydani, M., Hasan, S. T., Dietary polyphenols and obesity. Nutrients 2010, 2, 737-751.
5. Siriwardhana, N., Kalupahana, N. S., Cekanova, M., LeMieux, M., et al., Modulation of adipose tissue inflammation by bioactive food compounds. The Journal of nutritional biochemistry 2013, 24, 613-623.
6. Azhar, Y., Parmar, A., Miller, C. N., Samuels, J. S., Rayalam, S., Phytochemicals as novel agents for the induction of browning in white adipose tissue. Nutrition & metabolism 2016, 13, 89.
Wu, J., Cohen, P., Spiegelman, B. M., Adaptive thermogenesis in adipocytes: is beige the new brown? Genes & development 2013, 27, 234-250.
Mele, L., Bidault, G., Mena, P., Crozier, A., et al., Dietary (poly) phenols, brown adipose tissue activation, and energy expenditure: a narrative review. Advances in Nutrition 2017, 8, 694-704.
Zhang, H., Tsao, R., Dietary polyphenols, oxidative stress and antioxidant and anti-inflammatory effects. Current Opinion in Food Science 2016, 8, 33-42.
7. Kim, H., Simbo, S., Fang, C., McAlister, L., et al., Açai (Euterpe oleracea Mart.) beverage consumption improves biomarkers for inflammation but not glucose- or lipid-metabolism in individuals with metabolic syndrome in a randomized, double-blinded, placebo-controlled clinical trial. Food & function 2018.
8. Fang, C., Kim, H., Noratto, G., Sun, Y., et al., Gallotannin derivatives from mango (Mangifera indica L.) suppress adipogenesis and increase thermogenesis in 3T3-L1 adipocytes in part through the AM PK pathway. Journal of Functional Foods 2018, 46, 101-109.
9. Pandey, A., Bani, S., Sangwan, P. L., Anti-Obesity Potential of Gallic Acid from Labisia pumila, through Augmentation of Adipokines in High Fat Diet Induced Obesity in C57BL/6 Mice. Advan. Res 2014, 2, 556-570.
10. Fang, C., Kim, H., Barnes, R. C., Talcott, S. T., Mertens-Talcott, S. U., Obesity-Associated Diseases Biomarkers are Differently Modulated in lean and Obese Individuals and Inversely Correlated to Plasma Polyphenolic Metabolites After 6 Weeks of Mango (Mangifera Indica L.) Consumption. Molecular nutrition & food research 2018, 1800129.
11. Natal, D. I. G., de Castro Moreira, M. E., Milian, M. S., dos Anjos Benjamin, L., et al., Ubá mango juices intake decreases adiposity and inflammation in high-fat diet-induced obese Wistar rats. Nutrition 2016, 32, 1011-1018.
12. Lucas, E. A., Li, W., Peterson, S. K., Brown, A., et al., Mango modulates body fat and plasma glucose and lipids in mice fed a high-fat diet. British journal of nutrition 2011, 106, 1495-1505.
13. Taing, M.-W., Pierson, J.-T., Hoang, V. L., Shaw, P. N., et al., Mango fruit peel and flesh extracts affect adipogenesis in 3T3-L1 cells. Food & function 2012, 3, 828-836.
14. Evans, S. F., Meister, M., Mahmood, M., Eldoumi, H., et al., Mango supplementation improves blood glucose in obese individuals. Nutrition and metabolic insights 2014, 7, NMI. S17028.
15. Lamy, E., Pinheiro, C., Rodrigues, L., Capela-Silva, F., et al., Determinants of tannin-rich food and beverage consumption: oral perception vs. psychosocial aspects. 2016.
16. Cardona, F., Andres-Lacueva, C., Tulipani, S., Tinahones, F. J., Queipo-Orturio, M. I., Benefits of polyphenols on gut microbiota and implications in human health. The Journal of nutritional biochemistry 2013, 24, 1415-1422.
17. Aura, A.-M., Microbial metabolism of dietary phenolic compounds in the colon. Phytochemistry Reviews 2008, 7, 407-429.
18. Scalbert, A., Morand, C., Manach, C., Rémésy, C., Absorption and metabolism of polyphenols in the gut and impact on health. Biomedicine & Pharmacotherapy 2002, 56, 276-282.
19. Dallal, M. S., Zamaniahari, S., Davoodabadi, A., Hosseini, M., Rajabi, Z., Identification and characterization of probiotic lactic acid bacteria isolated from traditional persian pickled vegetables. GMS hygiene and infection control 2017, 12.
20. Niedzielin, K., Kordecki, H., ena Birkenfeld, B., A controlled, double-blind, randomized study on the efficacy of Lactobacillus plantarum 299V in patients with irritable bowel syndrome. European journal of gastroenterology & hepatology 2001, 13, 1143-1147.
21. Jiménez, N., Curiel, J. A., Reveron, I., de las Rivas, B., Muñoz, R., Uncovering the Lactobacillus plantarum WCFS1 gallate decarboxylase involved in tannin degradation. Applied and environmental microbiology 2013, 79, 4253-4263.
22. Jimenez, N., Esteban-Torres, M., Mancheño, J. M., de las Rivas, B., Munoz, R., Tannin degradation by a novel tannase enzyme present in some Lactobacillus plantarum strains. Applied and environmental microbiology 2014, 80, 2991-2997.
23. Pasinetti, G. M., Singh, R., Westfall, S., Herman, F., et al., The Role of the Gut Microbiota in the Metabolism of Polyphenols as Characterized by Gnotobiotic Mice. Journal of Alzheimer's Disease 2018, 1-13.
24. Strumeyer, D. H., Malin, M. J., Condensed tannins in grain sorghum. Isolation, fractionation, and characterization. Journal of Agricultural and Food Chemistry 1975, 23, 909-914.
25. Sirven, M. A., Negrete, M., Talcott, S. T., Tannase improves gallic acid bioaccessibility and maintains the quality of mango juice. International Journal of Food Science & Technology, 0.
26. Ayed, L., Hamdi, M., Culture conditions of tannase production by Lactobacillus plantarum. Biotechnology Letters 2002, 24, 1763-1765.
27. Reverón, I., de las Rivas, B., Matesanz, R., Munoz, R., de Felipe, F. L., Molecular adaptation of Lactobacillus plantarum WCFS1 to gallic acid revealed by genome-scale transcriptomic signature and physiological analysis. Microbial cell factories 2015, 14, 160.
28. Fontaine, C. A., Skorupski, A. M., Vowles, C. J., Anderson, N. E., et al., How free of germs is germ-free? Detection of bacterial contamination in a germ free mouse unit. Gut microbes 2015, 6, 225-233.
29. Chung, H., Pamp, S. J., Hill, J. A., Surana, N. K., et al., Gut immune maturation depends on colonization with a host-specific microbiota. Cell 2012, 149, 1578-1593.
30. Shoelson, S. E., Herrero, L., Naaz, A., Obesity, inflammation, and insulin resistance. Gastroenterology 2007, 132, 2169-2180.
31. Morris, J. L., Bridson, T. L., Alim, M. A., Rush, C. M., et al, Development of a diet-induced murine model of diabetes featuring cardinal metabolic and pathophysiological abnormalities of type 2 diabetes. Biology open 2016, 5, 1149-1162.
32. Menon, V., Zhi, X., Hossain, T., Bartke, A., et al., The contribution of visceral fat to improved insulin signaling in Ames dwarf mice. Aging cell 2014, 13, 497-506.
33. Wang, S., Liang, X., Yang, Q., Fu, X., et al., Resveratrol induces brown-like adipocyte formation in white fat through activation of AMP-activated protein kinase (AMPK) α1. International Journal of Obesity 2015, 39, 967.
34. Vázquez-Vela, M. E. F., Torres, N., Tovar, A. R., White adipose tissue as endocrine organ and its role in obesity. Archives of medical research 2008, 39, 715-728.
35. Moseti, D., Regassa, A., Kim, W.-K., Molecular regulation of adipogenesis and potential anti-adipogenic bioactive molecules. International journal of molecular sciences 2016, 17, 124.
36. Wu, L., Zhang, L., Li, B., Jiang, H., et al., AMP-Activated Protein Kinase (AMPK) Regulates Energy Metabolism through Modulating Thermogenesis in Adipose Tissue. Frontiers in physiology 2018, 9, 122.
37. van Dam, A. D., Kooijman, S., Schilperoort, M., Rensen, P. C., Boon, M. R., Regulation of brown fat by AMP-activated protein kinase. Trends in molecular medicine 2015, 21, 571-579.
38. Yamashita, Y., Okabe, M., Natsume, M., Ashida, H., Prevention mechanisms of glucose intolerance and obesity by cacao liquor procyanidin extract in high-fat diet-fed C57BL/6 mice. Archives of biochemistry and biophysics 2012, 527, 95-104.
39. Chen, L.-H., Chien, Y.-W., Liang, C.-T., Chan, C.-H., et al., Green tea extract induces genes related to browning of white adipose tissue and limits weight-gain in high energy diet-fed rat. Food & nutrition research 2017, 61, 1347480.
40. Lee, M.-S., Shin, Y., Jung, S., Kim, Y., Effects of epigallocatechin-3-gallate on thermogenesis and mitochondrial biogenesis in brown adipose tissues of diet-induced obese mice. Food & nutrition research 2017, 61, 1325307.
41. Aguirre, L., Fernandez-Quintela, A., Arias, N., Portillo, M. P., Resveratrol: anti-obesity mechanisms of action. Molecules 2014, 19, 18632-18655.
42. Forney, L. A., Lenard, N. R., Stewart, L. K., Henagan, T. M., Dietary Quercetin Attenuates Adipose Tissue Expansion and Inflammation and Alters Adipocyte Morphology in a Tissue-Specific Manner. International journal of molecular sciences 2018, 19, 895.
43. Han, Y., Wu, J.-Z., Shen, J.-z., Chen, L., et al., Pentamethylquercetin induces adipose browning and exerts beneficial effects in 3T3-L1 adipocytes and high-fat diet-fed mice. Scientific reports 2017, 7, 1123.
44. Song, Z., Revelo, X., Shao, W., Tian, L., et al., Dietary Curcumin Intervention Targets Mouse White Adipose Tissue Inflammation and Brown Adipose Tissue UCP1 Expression. Obesity 2018, 26, 547-558.
45. Ejaz, A., Wu, D., Kwan, P., Meydani, M., Curcumin inhibits adipogenesis in 3T3-L1 adipocytes and angiogenesis and obesity in C57/BL mice. The Journal of nutrition 2009, 139, 919-925.
46. Asai, A., Miyazawa, T., Dietary curcuminoids prevent high-fat diet-induced lipid accumulation in rat liver and epididymal adipose tissue. The Journal of nutrition 2001, 131, 2932-2935.
47. Nemec, M. J., Kim, H., Marciante, A. B., Barnes, R. C., et al., Polyphenolics from mango (Mangifera indica L.) suppress breast cancer ductal carcinoma in situ proliferation through activation of AMPK pathway and suppression of mTOR in athymic nude mice. The Journal of nutritional biochemistry 2017, 41, 12-19.
48. Kim, H., Banerjee, N., Ivanov, I., Pfent, C. M., et al., Comparison of anti-inflammatory mechanisms of mango (Mangifera Indica L.) and pomegranate (Punica Granatum L.) in a preclinical model of colitis. Molecular nutrition & food research 2016, 60, 1912-1923.
49. Kim, H., Banerjee, N., Barnes, R. C., Pfent, C. M., et al., Mango polyphenolics reduce inflammation in intestinal colitis—involvement of the miR-126/PI3K/AKT/mTOR axis in vitro and in vivo. Molecular carcinogenesis 2017, 56, 197-207.
50. Yang, C. S., Wang, H., Sheridan, Z. P., Studies on prevention of obesity, metabolic syndrome, diabetes, cardiovascular diseases and cancer by tea. journal of food and drug analysis 2017.
51. Moreno-Indies, I., Sanchez-Alcoholado, L., Perez-Martinez, P., Andres-Lacueva, C., et al., Red wine polyphenols modulate fecal microbiota and reduce markers of the metabolic syndrome in obese patients. Food & function 2016, 7, 1775-1787.
52. Tzounis, X., Rodriguez-Mateos, A., Vulevic, J., Gibson, G. R., et al., Prebiotic evaluation of cocoa-derived flavanols in healthy humans by using a randomized, controlled, double-blind, crossover intervention study—. The American journal of clinical nutrition 2010, 93, 62-72.
53. Molan, A.-L., Liu, Z., Kruger, M., The ability of blackcurrant extracts to positively modulate key markers of gastrointestinal function in rats. World Journal of Microbiology and Biotechnology 2010, 26, 1735-1743.
54. Viveros, A., Chamorro, S., Pizarro, M., Arija, I., et al., Effects of dietary polyphenol-rich grape products on intestinal microflora and gut morphology in broiler chicks. Poultry science 2011, 90, 566-578.
55. Vaquero, I., Marcobal, Á., Muñoz, R., Tannase activity by lactic acid bacteria isolated from grape must and wine. International journal of food microbiology 2004, 96, 199-204.
56. Barnes, R. C., Kim, H., Fang, C., Bennett, W., Nemec, M., Sirven, M. A., Suchodolski, J. S., Deutz, N., Britton, R., M.-T., S. U. & Talcott, S. T., Body Mass Index as a determinant of systemic exposures to gallotannin metabolites during six-week consumption of mango (Mangifera Indica L.) and modulation of intestinal microbiota in lean and obese individuals. Molecular nutrition & food research 2018, In Press.
57. Axling, U., Olsson, C., Xu, J., Fernandez, C., et al., Green tea powder and Lactobacillus plantarum affect gut microbiota, lipid metabolism and inflammation in high-fat fed C57BU6J mice. Nutrition & metabolism 2012, 9, 105.
58. WulIt, M., Hagslätt, M.-L. J., Odenholt, I., Berggren, A., Lactobacillus plantarum 299v enhances the concentrations of fecal short-chain fatty acids in patients with recurrent Clostridium difficile-associated diarrhea. Digestive diseases and sciences 2007, 52, 2082.
59. Brahe, L. K., Astrup, A., Larsen, L. H., Is butyrate the link between diet, intestinal microbiota and obesity-related metabolic diseases? Obesity reviews 2013, 14, 950-959.

### Example 6: Synbiotic Interactions of Probiotics, Tannase, and Mango Polyphenols in Improving Absorption of Anti-Inflammatory Metabolites, Intestinal Health, and Cognitive Function in Obese Adolescents

The US currently has over 100 million obese adults and over 12 million obese children (aged 0-18) [1]. This population is known to suffer from a dysbiotic intestinal microbiota [2, 3] and these individuals may not optimally benefit from the intake of fruits and vegetables as their lean counterparts due to the limited production of microbial absorbable beneficial polyphenol metabolites [4]. Obesity rates are higher than the national average for African-American adults (48%) and Hispanic adults (42%), and projections for adolescents are expected to exceed 50% in the near future [1, 5]. As in adults, in adolescents, obesity is associated with intestinal dysbiosis, reduced cognitive function, and increased inflammation which may lead to lower academic performance which may be specifically crucial for individuals of late middleschool and highschool age where school performance often reflects into adulthood [2, 3]. For this reason, the proposed study is targeting adolescents of late middleschool to highschool age (13-17 years) including hispanic adolescents.

Dysbiosis, Tannase, and Probiotics

Our research indicates that for obese individuals, adding mangoes to the diet does result in the improvement of biomarkers for inflammation and improves intestinal dysbiosis in lean and obese individuals, but the absorption and over-time increase in absorbed beneficial metabolites is much lower on obese compared to lean individuals [4, 6-10]. For this reason, in a currently ongoing study in adults, probiotics are included to improve the intestinal microbiota and over time increase the absorption of beneficial gallotannin metabolites.

This research approach offers an additional step to yield early absorbable metabolites and provide smaller prebiotics to intestinal probiotic bacteria: Pre-hydrolysis of mango gallotannins using tannase (tannin acyl hydrolase, E.C. 3.1.1.1.20) which is a food-grade processing aid commonly used in the food industry (e.g. in the production of green and black teas). The enzyme catalyzes ester-bonds between acid and hydroxyl moieties on polyphenols and m-depside bonds between phenolic acids and phenolic hydroxyls. An individual's bacterial population and activity of tannase they express will vary greatly. As a remedy, we have identified a tannase from Aspergillus oryzae that remains active during gastric digestion and remains active in the small intestine. We have conducted significant preliminary work to understand the role of supplemented tannase during digestion. While the use of probiotics is a long-term solution to overcome chronic inflammation, addition of tannase that can function in both the stomach and small intestines is a faster-response to gallotannin metabolism and produces metabolites earlier in the consumption and digestive process.

This concept in principle is well established for example for individuals who are not able to digest legumes or dairy products without digestive discomfort where an enzyme prior to eating beans (alpha-galactosidase) or milk (beta-galactosidase/lactase) can be used to aid digestion. Our preliminary data show that tannase added to gallotannins will survive oral and gastric digestion to create gallic acid monomer and smaller oligomers that can be further digested by probiotic bacteria. Therefore, this strategy optimizes the benefits of mango gallotannins in obese individuals.

Overall, the proposed research will overcome the known limited absorption of gallotannin metabolites in obese individuals by pre-digestion with tannase to form smaller molecules and adding probiotics for intestinal digestion of gallotannins.

Gut-Brain Axis

Several studies have identified the relationship between obesity and chronic inflammation [11, 12]. A novel and exciting area of research seeks to comprehend interactions within the gut-brain axis that involves the intestinal microbiome and its crucial role in systemic inflammation and cognitive function. Changes in the gut microbiota composition can modulate glucose tolerance and lipoprotein profile, and inflammation [13]. The number of investigations of the gut-brain axis is growing but clinical approaches in this area are still lacking.

Cognitive Function

Cognitive functions are defined as cerebral activities including reasoning, memory, attention, and language, and are directly related to the attainment of information and, thus, knowledge. Although decreased cognitive function may be associated with normal aging, it can also lead to the development of diseases such as dementia and Alzheimer disease [14, 15]. While stress and diabetes can contribute to cognitive impairment [15, 16], phytochemicals and vitamins found in fruits and vegetables have been found to improve different aspects of cognitive function. Haskell and collaborators [17] found that a multi-vitamin/mineral supplementation were able to complete mathematical problems faster and more accurately than subjects who received placebo. A positive correlation was also found for vitamin E and C supplementation and increased cognitive function by Masaki et al [18]. Since experimental data indicate reactive oxygen species may be involved in the deterioration of the cognitive process [19], it is possible that polyphenol-rich fruits could be beneficial to the population by increasing their response to memory, attention, and concentration. Phytochemical studies demonstrate that polyphenols and other phytochemicals can beneficially influence cognitive function in animals and humans.

In our past human trial comparing lean and obese cohorts consuming mango gallotannins continuously for 42-days we observed that obese subjects expressed significantly less tannase in their feces compared to lean subjects and had lower plasma and urinary metabolite concentrations and produced fewer short-chain fatty acids. However, after 42 days, obese subjects increased levels of tannase-producing Lactoccoccus lactis and decreased levels of Clostridium leptum and Bacteroides thetaiotaomicron, strains generally associated with obesity and intestinal toxins [4], [6]. Our currently ongoing clinical trial shows that cognitive function is improved in adult individuals consuming mangoes. In the in vitro digestive model (oral-gastric-pancreatic digestions) tannase showed its action on gallotannin pentagalloyl glucose. Tannase remained active during the oral and gastric phase, and increased activity in the intestinal phase, showing that the enzyme survives these digestive conditions. At very low tannase concentrations (up to 20 U/gram) and after only 1 h of digestion, tannase increase free gallic acid 10-fold and produce multiple oligomers including two tetragalloyl glucoses, six trigalloyl glucoses, and five digalloyl glucose isomers that can serve as prebiotics.

Clinical Trials

This randomized, human clinical trial will be performed over 8 weeks at the human clinical laboratories at Texas A&M University under the supervision of our research nurses Sandra Miller RN and Cathy Craig, RN and the study physician. The clinical study will be designed as a randomized, trial in obese adolescent teenagers. The study will be carried out after approval by the Institutional Review Boards (IRB) at Texas A&M University and will be registered at www.clinicaltrials.gov upon initiation.

Participants will be recruited in collaboration with science teachers at private and public middle and high schools in Brazos county, TX. Obese teens (BMI 27 to 35), male or female (13-17 years), n=48 (24 per study group). Exclusion criteria: History of acute cardiac event, stroke, or cancer, within the last 6 months, recurrent hospitalizations, drug treatment of any of the listed conditions within the last 6 months, abuse of alcohol or substance within the last 6 months, currently smoking more than 1 pack/week, seizures, liver or renal dysfunction, pregnancy or lactation, allergy against mangoes, probiotics, hepatitis B, C, or HIV.

Mango: Study subjects will receive a periodic supply of individually sealed and frozen bags of mango, preferably Ataulfo, to consumed at a rate of 250 g/day for 8 weeks. We hypothesize that this synbiotic approach in the study of adolescents will allow a smaller, more manageable serving of mango for this study population (250 g/day or one medium sized mango instead of 400 g, as previously administered to adults). Control/Placebo: One half of the randomized participants will consume only mango and a placebo capsule. Treatment: The other half will receive 250 g/day of mango that contains 250 mg of a commercial tannase (5 enzyme units/g) added to fruit and subjects will also consume a capsule containing a probiotic mixture of FDA-approved probiotic bacteria that are positive for gallotannin metabolism including Lactobacillus casei, Lactobacillus rhamnosus, Lactobacillus plantarum, Bifidobacterium bifidum, and Bifidobacterium breve.

A total of three sample collection sessions (Day 1, after 4 weeks and 8 weeks) are planned. Three days prior to when the study begins, subjects will be asked to refrain from consuming foods known to contain gallic acid or pro-gallic acid polyphenolics including mango, grape products, tea, chocolate, and berries. Before each study day, subjects will be asked to stop taking nutritional supplements (one week before), avoid excessive exercise (72 hours before), and fast (only drink water, 12 hours before). Blood will be collected at 0 hours (baseline before the administration of study treatment) and at 2 hrs on study days. Before the first study day and at 8-weeks participants will collect urine and stool samples at baseline and after 8-weeks. Samples will be aliquoted and stored at −80° C. until analysis.

Sample Analysis

Polyphenolic metabolites will be extracted from urine using solid phase extraction and urine microalbumin-to-creatinine ratios will be determined to normalize mango metabolites. Blood serum processing and metabolomic profiling will be conducted according to a novel analysis method developed specifically for gallotannin metabolites, as previously reported. Short-chain fatty acids will be extracted from fecal samples with organic solvent and analyzed after centrifugation. Analysis will include both targeted (known metabolites) and un-targeted metabolite modeling allowing for identification and quantitation of mango-derived metabolites. Thanks to investments in a new “Food Forensics Laboratory” in the Department of Nutrition and Food Science under the direction of Drs. Steve and Susanne Talcott, we will be able to apply the latest advancements in HPLC and GC mass spectroscopy testing to these samples. Analysis will be based on fold-changes for individual analytes relative to mango baseline and tannase+probiotic treatment. This will allow for comparison of both individual metabolite profiles as well as total absorption/excretion between the control and treatment effect established using linear mixed model ANOVA and appropriate post-hoc test as determined by data normality (SAS version 9.4; SAS Institute). We expect to detect an increase in systemic polyphenol metabolite concentrations after 8 weeks of consumption specifically with the tannase/probiotics treatment.

Executive attention, and visuospatial functions will be assessed by the Trail Making Test (TMT A and B) and Wechsler Scale-Revised Digit Span. Their progress on each test will be followed throughout the study on each study day (days 1, 22, and 43). The trail-making test assesses measure of attention, speed, and mental flexibility, spatial organization, visual pursuits, recall, and recognition. The Wechsler Scale-Revised Digit Span is a measure of mental tracking as well as of memory and mental flexibility [20, 21]. Additionally, the 3D Neurotracker will be used to evaluate 3D orientation that will evaluate the special awareness of individuals that is used as an indicator for spatial awareness (clumsiness) and the ability to follow objects in 3D space under the direction of Dr. Stephen Riechman, Health and Kinesiology/Psychology Department Texas A&M University. The latter is highly sensitive and able to detect minor changes in diet, so we anticipate that mango treatments, specifically with tannase/probiotics, will be able to induce significant changes in these measures.

Blood Cell Activation: White blood cells will be activated with inflammatory physiological molecules and the inflammatory response will be measured. Inflammation Biomarkers: A panel of biomarkers associated with cardiovascular metabolism will be assessed in the plasma of each participant using xMAP Multiplex technology (Luminex 200, Luminex Corporation, Austin, Tex., USA) as previously performed [5, 6]. We expect inflammation markers to decrease after 8 weeks of mango consumption specifically with the tannase/probiotic treatment.

Microbial Analysis: qPCR targeting 16S rRNA genes is a useful tool for quantifying very low concentrations of bacterial targets in fecal samples [22-24]. We expect to see beneficial changes in the intestinal microbiota composition, specifically in the group treatment with tannase/probiotics.

### References for Example 6

1. Skinner A C, Ravanbakht S N, Skelton J A, Perrin E M, Armstrong S C: Prevalence of Obesity and Severe Obesity in US Children, 1999-2016. Pediatrics 2018, 141(3).
2. Afzal A S, Gortmaker S: The Relationship between Obesity and Cognitive Performance in Children: A Longitudinal Study. Childhood obesity (Print) 2015, 11(4):466-474.
3. Brusaferro A, Cavalli E, Farinelli E, Cozzali R, Principi N, Esposito S: Gut dysbiosis and paediatric Crohn's disease. The Journal of infection 2019, 78(1):1-7.
4. Barnes R C, Kim H, Fang C, Bennett W, Nemec M, Sirven M A, Suchodolski J S, Deutz N, Britton R A, Mertens-Talcott S U et al: Body Mass Index as a Determinant of Systemic Exposure to Gallotannin Metabolites during 6-Week Consumption of Mango (Mangifera indica L.) and Modulation of Intestinal Microbiota in Lean and Obese Individuals. Molecular Nutrition and Food Research 2019, 63(2).
5. Cheung P C, Cunningham S A, Narayan K M, Kramer M R: Childhood Obesity Incidence in the United States: A Systematic Review. Childhood obesity (Print) 2016, 12(1):1-11.
6. Fang C, Kim H, Barnes R C, Talcott S T, Mertens-Talcott S U: Obesity-Associated Diseases Biomarkers Are Differently Modulated in Lean and Obese Individuals and Inversely Correlated to Plasma Polyphenolic Metabolites After 6 Weeks of Mango (Mangifera indica L.) Consumption. Molecular Nutrition and Food Research 2018, 62(14).
7. Fang C, Kim H, Noratto G, Sun Y, Talcott S T, Mertens-Talcott S U: Gallotannin derivatives from mango (Mangifera indica L.) suppress adipogenesis and increase thermogenesis in 3T3-L1 adipocytes in part through the AMPK pathway. Journal of Functional Foods 2018, 46:101-109.
8. Kim H, Banerjee N, Barnes R C, Pfent C M, Talcott S T, Dashwood R H, Mertens-Talcott S U: Mango polyphenolics reduce inflammation in intestinal colitis—involvement of the miR-126/PI3K/AKT/mTOR axis in vitro and in vivo. Molecular Carcinogenesis 2017, 56(1):197-207.
9. Kim H, Krenek K A, Fang C, Minamoto Y, Markel M E, Suchodolski J S, Talcott S T, Mertens-Talcott S U: Polyphenolic derivatives from mango (Mangifera Indica L.) modulate fecal microbiome, short-chain fatty acids production and the HDAC1/AMPK/LC3 axis in rats with DSS-induced colitis. Journal of Functional Foods 2018, 48:243-251.
10. Venancio V P, Kim H, Sirven M A, Tekwe C D, Honvoh G, Talcott S T, Mertens-Talcott S U: Polyphenol-rich Mango (Mangifera indica L.) Ameliorate Functional Constipation Symptoms in Humans beyond Equivalent Amount of Fiber. Molecular Nutrition and Food Research 2018, 62(12).
11. Tome-Carneiro J, Visioli F: Polyphenol-based nutraceuticals for the prevention and treatment of cardiovascular disease: Review of human evidence. Phytomedicine 2016, 23(11):1145-1174.
12. Guo X, Tresserra-Rimbau A, Estruch R, Martinez-Gonzalez M A, Medina-Remon A, Castaner O, Corella D, Salas-Salvado J, Lamuela-Raventos R M: Effects of Polyphenol, Measured by a Biomarker of Total Polyphenols in Urine, on Cardiovascular Risk Factors After a Long-Term Follow-Up in the PREDIMED Study. Oxid Med Cell Longev 2016, 2016:2572606.
13. Rune I, Rolin B, Larsen C, Nielsen D S, Kanter J E, Bornfeldt K E, Lykkesfeldt J, Buschard K, Kirk R K, Christoffersen B et al: Modulating the Gut Microbiota Improves Glucose Tolerance, Lipoprotein Profile and Atherosclerotic Plaque Development in ApoE-Deficient Mice. PLoS One 2016, 11(1):e0146439.
14. Carlson J J, Farquhar J W, DiNucci E, Ausserer L, Zehnder J, Miller D, Berra K, Hagerty L, Haskell W L: Safety and efficacy of a Ginkgo biloba-containing dietary supplement on cognitive function, quality of life, and platelet function in healthy, cognitively intact older adults. J Am Diet Assoc 2007, 107(3):422-432.
15. Arvanitakis Z, Wilson R S, Bienias J L, Evans D A, Bennett D A: Diabetes mellitus and risk of Alzheimer disease and decline in cognitive function. Arch Neurol 2004, 61(5):661-666.
16. McEwen B S, Sapolsky R M: Stress and cognitive function. Curr Opin Neurobiol 1995, 5(2):205-216.
17. Haskell C F, Robertson B, Jones E, Forster J, Jones R, Wilde A, Maggini S, Kennedy D O: Effects of a multi-vitamin/mineral supplement on cognitive function and fatigue during extended multi-tasking. Hurn Psychopharmacol 2010, 25(6):448-461.
18. Masaki K H, Losonczy K G, lzmirlian G, Foley D J, Ross G W, Petrovitch H, Havlik R, White L R: Association of vitamin E and C supplement use with cognitive function and dementia in elderly men. Neurology 2000, 54(6):1265-1272.
19. Grodstein F, Chen J, Willett W C: High-dose antioxidant supplements and cognitive function in community-dwelling elderly women. Am J Clin Nutr 2003, 77(4):975-984.
20. Carlesimo G A, Caltagirone C, Gainotti G: The Mental Deterioration Battery: normative data, diagnostic reliability and qualitative analyses of cognitive impairment. The Group for the Standardization of the Mental Deterioration Battery. Eur Neurol 1996, 36(6):378-384.
21. Rizzo M R, Barbieri M, Boccardi V, Angellotti E, Marfella R, Paolisso G: Dipeptidyl peptidase-4 inhibitors have protective effect on cognitive impairment in aged diabetic patients with mild cognitive impairment. J Gerontol A Biol Sci Med Sci 2014, 69(9):1122-1131.
22. Suchodolski J S, Xenoulis P G, Paddock C G, Steiner J M, Jergens A E: Molecular analysis of the bacterial microbiota in duodenal biopsies from dogs with idiopathic inflammatory bowel disease. Veterinary microbiology 2010, 142(3-4):394-400.
23. Jimenez N, Esteban-Torres M, Mancheno J M, de Las Rivas B, Munoz R: Tannin degradation by a novel tannase enzyme present in some Lactobacillus plantarum strains. Applied and environmental microbiology 2014, 80(10):2991-2997.
24. Garcia-Mazcorro J F, Suchodolski J S, Jones K R, Clark-Price S C, Dowd S E, Minamoto Y, Markel M, Steiner J M, Dossin 0: Effect of the proton pump inhibitor omeprazole on the gastrointestinal bacterial microbiota of healthy dogs. FEMS Microbiol Ecol 2012, 80(3):624-636.
25. Fang C, Kim H, Yanagisawa L, Bennett W, Sirven M A, Alaniz R C, Talcott S T, Mertens-Talcott S U: Gallotannins and Lactobacillus plantarum WCFS1 Mitigate High-Fat Diet-Induced Inflammation and Induce Biomarkers for Thermogenesis in Adipose Tissue in Gnotobiotic Mice. Molecular Nutrition and Food Research 2019.

### Example 7: Enhancing the Efficacy of Mango Phytochemicals in Cognitive Function and Cardiometabolic Health in Lean and Obese Subjects

Our research team has been involved with mango polyphenol research over the last decade and investigated chemical and health-related properties. In our previous studies we demonstrated that obese individuals are exposed to lower concentrations of mango polyphenol metabolites upon mango consumption when compared to lean individuals. Additionally, we showed that mango gallotannins present a specific substrate to beneficial bacteria that increase the metabolism of large gallotannins into small beneficial absorbable molecules. Therefore, the administration of probiotic bacteria is currently being investigated in the optimization of cognitive function, inflammation (immune cell activation). In this study, blood, urine, stool and lipid biopsies are collected for analysis in this proposed phase II of this project where the influence of mango with and without probiotics on absorption of polyphenols and carotenoids, and cardiometabolic markers will be investigated.

Our previous studies show that individuals absorb more mango polyphenols after consuming mango for several weeks (lean individuals but not the obese) and we learned that the beneficial polyphenol metabolites can be present in the body even after 48 h. For this reason, absorption parameters will be investigated in samples collected after 24, 48 and 72 h after consumption in the beginning middle and end of the overall study. Additionally, the presence of phytochemicals in certain blood cell compartments is a predictor of their presence in certain tissues (e.g. heart and brain). For this reason, the absorption of polyphenols and carotenoids will be evaluated in blood cells and lipoprotein fractions. Concentrations and location of metabolites will be correlated to biomarkers for cardiometabolic health and cognitive function. Overall, polyphenols, such as flavan-3-ols, flavonols and anthocyanins, and is overall widely unexplored and the fasted growing area of phytochemical research today. Our research team has demonstrated the ability to effectively perform targeted and untargeted metabolite analysis of polyphenols and carotenoids, perform human clinical trials with mangoes analyzing multiple biomarkers.

Polyphenolics identified in the edible part of mango (Mangifera indica) have been previously characterized and include flavonoids such as quercetin and kaempferol glycosides, phenolics acids, predominantly gallic acid, galloyl glycosides, in part polymerized, and in some varieties mangiferin [1]. Overall, cytotoxic [1] and anti-inflammatory effects [2] of polyphenolics from mango have been investigated, where a comparison of several mango varieties in their cytotoxic activities in different cancer cell lines by our research group demonstrated the colon-cancer-cytotoxic and anti-inflammatory activities in vitro [3]. Multiple studies have demonstrated the health benefits of secondary plant compounds in fruits and vegetables including pomegranate, citrus, and curcuminoids. Polyphenolics from mango reduce inflammatory processes relevant to many chronic diseases, such as cardiovascular disease, cancer [4], and inflammatory bowel disease [5].

Cardio-metabolic health: Several studies have identified the relationship between diet and the development of cardiovascular diseases (CVD); polyphenol-based diets have directly been correlated to the reduction of cardiovascular disease and cardiac failure [6, 7]. A novel and exciting area of research seeks to comprehend interactions within the gut-heart-brain axis that involves the intestinal microbiome and its crucial role in diabetes, cardiovascular diseases, and obesity. Changes in the gut microbiota composition can modulate glucose tolerance and lipoprotein profile, modulating the risk of CVD [8]. In the same way, researchers believe that modulating gut-brain signaling is important in governing energy homeostasis and metabolism [9]. The number of investigations of the gut-heart-brain axis is growing but clinical approaches in this area are still lacking. Therefore, investigating metabolic markers such as apolipoproteins and gut endocrine factors (such as peptide YY and ghrelin) becomes useful as a tool to understand the relationship between the cardiovascular system, the metabolism, and the gut microbiome.

This randomized, human clinical trial is currently performed over 8 weeks at the human clinical laboratories at Texas A&M University under the supervision of a research nurse and study physician. The clinical study is designed as a randomized, trial in healthy lean and obese subjects. The study is carried out after approval by the Institutional Review Boards (IRB) at Texas A&M University and is registered at www.clinicaltrials.gov. Cognitive function and blood cell activation (inflammation) is currently been evaluated.

Study population: Lean and obese subjects (BMI 18-23 or 27-35), male or female ages 18-65 years. Exclusion criteria: History of acute cardiac event, stroke, or cancer, within the last 6 months, recurrent hospitalizations, drug treatment of any of the listed conditions within the last 6 months, abuse of alcohol or substance within the last 6 months, currently smoking more than 1 pack/week, seizures, liver or renal dysfunction, pregnancy or lactation, allergy against mangoes, hepatitis B, C, or HIV.

Study Treatment: Mango: Study subjects receive fresh or freshly frozen edible portion of mango (var. Ataulfo), 400 g/day for 8 weeks.

Probiotics: one group each of lean and obese individuals are treated with a mixture of probiotic bacteria (FDA approved as dietary supplement) that are known to possess gallotannin-metabolizing enzymes, including Lactobacillus casei, Lactobacillus rhamnosus, Lactobacillus plantarum, Bifidobacterium bifidum, and Bifidobacterium breve.

Sample Collection: A total of three sample collection sessions (Days 1, after 4 weeks and 8 weeks) are taking place. Three days prior to study begin, subjects are asked to refrain from consuming foods known to contain gallic acid or pro-gallic acid polyphenolics including mango, grape products, tea, chocolate, and berries. Before each study day, subjects are asked to stop taking nutritional supplements (one week before), avoid excessive alcohol and exercise (72 hours before), fast (only drink water, 12 hours before).

Blood collection: 0 hours (baseline before the administration of study treatment), 24, 48 h, 72 h on study days. Blood fractionation. Fresh blood collected in EDTA tubes are centrifuged at 2000×g for 10 minutes to separate RBC, buffy coat (leucocytes and platelets) and plasma. 1 mL aliquots of RBC and plasma are collected, acidified with 0.1% formic acid and stored at −800 C. Buffy coat are collected for isolation of monocytes and platelet rich fractions as described by Dhurat & Sukesh (2014). Remaining fresh plasma are used for isolation of lipoproteins, including chylomicron/VLDL, LDL and HDL by sequential ultracentrifugation.

Blood Carotenoid Analysis. Carotenoids will be determined in blood fractions as described by Goltz et al (2012) with minor modification. Briefly, ˜100-200 mL of blood serum will be thawed and deproteinated by addition of 100 mL of ice cold methanol (0.01% BHT). Samples are then extracted three times with 3 mL of acetone:petroleum ether (1:2).

Urine and stool collections: On Weeks 0 and 8, all urine produced throughout 72 hours will be stored in appropriate containers, following the following schedule: 0-24, 24-48, 48-72 h. Urine samples will be acidified, aliquoted immediately after obtained, and stored at −80° C. until analysis. Subjects will be provided with stool collection kits and asked to donate one stool sample on each study day (Weeks 0, 4, and 8). Samples will be aliquoted and stored at −80° C. until analysis.

Lipid Biopsies: A lipid biopsy (needle aspiration) will be collected from a subset of participants by the research nurse at 0 and 8 weeks to evaluate the effects of mango on fat metabolism within lipid tissue.

Chemical Analysis of polyphenols and carotenoids: Mass spectrometry for polyphenol metabolites in blood fractions and urine: Metabolites will be extracted from urine and plasma using Solid phase extraction and urine microalbumin-to-creatinine ratios will be determined to normalize mango metabolites. Following solid phase extraction (Strata X micro-elution; Phenomenex), Blood serum processing and metabolomic profiling: Analysis includes both targeted (previously established) and semi/un-targeted predictive metabolite modeling allowing for identification and quantitation of known and putative metabolites. Our present methods (1-4) are optimized for the extraction, identification and quantification of over 250 unconjugated and 50 conjugated metabolites with fragmentation profiling of 700+ transitions. The LC-MS/MS analysis is performed using an ExionLC AD UHPLC system coupled with a SCIEX 6500+MS/MS-QTRAP equipped with an electrospray ionization (ESI) IonDrive Turbo-V source and samples resolved by a Kinetex PFP column. The primary outcome from the targeted-metabolomic profiling will be qualitative and quantitative urinary phenolic metabolite recovery. Based on variance (30%) for total urinary recovery of phenolic metabolites observed in our studies(1-4) a sample size of 23 per group will be needed to detect a 25% change in total urinary phenolic metabolite excretions with 80% power (a=0.05). For the clinical intervention: We will establish total recovery in urine for each target phytochemical and metabolite over the treatment period. Analysis will be based on fold change for individual analytes relative to baseline and control ratios will be calculated to express differences in profiles between treatments as previously demonstrated. This will allow for comparison of both individual metabolite profiles as well as total absorption/excretion between the treatments and control for treatment effect established using linear mixed model ANOVA and appropriate post-hoc test as determined by data normality (SAS version 9.4; SAS Institute).

Carotenoids will be analyzed from ether layers; these are dried down and resolubilized in 1:1 methanol:ethyl acetate prior to analysis by LC-PDA as described by Lipkie et al. (2014).

Cardiometabolic Biomarkers: A panel of biomarkers associated with cardiovascular metabolism will be assessed in the plasma of each subject using xMAP Multiplex technology (Luminex 200, Luminex Corporation, Austin, Tex., USA) using magnetic beads acquired from EMD Millipore (Billerica, Mass., USA). Biomarkers include peptide YY (PYY), glucagon-like peptice-1 (GLP-1), gastric inhibitory polypeptide (GIP), ghrelin, apolipoprotein A1 (ApoA1), and apolipoprotein E (ApoE). The concentration of each biomarker will be calculated using standard curves and expressed as pg/mL. Biomarkers for lipid metabolism will be assessed in lipid tissue.

Microbial Analysis: Bacterial DNA (200 mg) will be extracted from fecal samples using a commercial DNA extraction kit (QIAGEN, Germany) according to the manufacturer's instructions [10]. Preliminary qPCR assays for selected bacterial groups will be performed: total bacteria (341F, 518R), Lactobacillus spp., Lactobacillus plantarum, Lactobacillus reuter, Lactococcus lactis [11]. The qPCR data will be expressed as log amount of DNA (fg) for each particular bacterial group [12]. Metagenomic analysis including whole genome shotgun sequencing and RNA-seq will be performed on the various mouse groups at same sampling times by Dr. Britton's laboratory. Reads will be mapped to MetaPhlAn markers for bacterial species classification [13] and to the KEGG database for functional gene annotation to identify candidate tannases and decarboxylases [14]. We will correlate specific metabolomic features (tannins and their metabolites, and SCFAs) with specific microbial communities that are known to possess one or both of these enzymes (Lactobacilli, Bifidobacterium, Ruminococci, Eubacteria, and Proteobacteria) in each subject using an orthogonalized partial least squares (OPLS) analysis [15]. Metabolomic features that were most sensitive to treatments using OPLS discriminant analysis within the treatment group will be used [16].

Statistical Analysis will be carried out in collaboration with Dr. Zhao. All statistical testing will be performed at the 0.05 level and will be two-sided. Data quality checks will be implemented, and data will be transformed as required. Analysis will be done on the intention-to-treat (ITT) principle. We will use analysis of variance (ANOVA) for crossover study designs, with Tukey or Wilcoxon rank test post-hoc analysis were appropriate. The absolute change from baseline will be tested and secondary analyses will be performed based upon significant change as established by treatment effect and time by treatment interaction. The randomization will be done in blocks with the block-numbers being mixed to protect the validity of the randomization. Block randomization with random block sizes preserves balance in treatment assignment, eliminates possible biases due to secular trends in recruitment, and preserves blinding [31]. Study participants will be randomized using stratification based on their gender and age using SAS (SAS Institute Inc., Cary, N.C.).

### References for Example 7

1. Rajendran P, Ekambaram G, Sakthisekaran D: Protective role of mangiferin against Benzo(a)pyrene induced lung carcinogenesis in experimental animals. Biol Pharm Bull 2008, 31(6):1053-1058.
2. Marquez L, Garcia-Bueno B, Madrigal J L, Leza J C: Mangiferin decreases inflammation and oxidative damage in rat brain after stress. European journal of nutrition 2012, 51(6):729-739.
3. Noratto G D, Bertoldi M C, Krenek K, Talcott S T, Stringheta P C, Mertens-Talcott S U: Anticarcinogenic effects of polyphenolics from mango (Mangifera indica) varieties. J Agric Food Chem 2010, 58(7):4104-4112.
4. Scalbert A, Manach C, Morand C, Remesy C, Jimenez L: Dietary polyphenols and the prevention of diseases. Crit Rev Food Sci Nutr 2005, 45(4):287-306.
5. Romier B, Schneider Y J, Larondelle Y, During A: Dietary polyphenols can modulate the intestinal inflammatory response. Nutr Rev 2009, 67(7):363-378.
6. Tome-Carneiro J, Visioli F: Polyphenol-based nutraceuticals for the prevention and treatment of cardiovascular disease: Review of human evidence. Phytomedicine 2016, 23(11):1145-1174.
7. Guo X, Tresserra-Rimbau A, Estruch R, Martinez-Gonzalez M A, Medina-Remon A, Castaner O, Corella D, Salas-Salvado J, Lamuela-Raventos R M: Effects of Polyphenol, Measured by a Biomarker of Total Polyphenols in Urine, on Cardiovascular Risk Factors After a Long-Term Follow-Up in the PREDIMED Study. Oxid Med Cell Longev 2016, 2016:2572606.
8. Rune I, Rolin B, Larsen C, Nielsen D S, Kanter J E, Bornfeldt K E, Lykkesfeldt J, Buschard K, Kirk R K, Christoffersen B et al: Modulating the Gut Microbiota Improves Glucose Tolerance, Lipoprotein Profile and Atherosclerotic Plaque Development in ApoE-Deficient Mice. PLoS One 2016, 11(1):e0146439.
9. Clemmensen C, Muller T D, Woods S C, Berthoud H R, Seeley R J, Tschop M H: Gut-Brain Cross-Talk in Metabolic Control. Cell 2017, 168(5):758-774.
10. Suchodolski J S, Xenoulis P G, Paddock C G, Steiner J M, Jergens A E: Molecular analysis of the bacterial microbiota in duodenal biopsies from dogs with idiopathic inflammatory bowel disease. Veterinary microbiology 2010, 142(3-4):394-400.
11. Jimenez N, Esteban-Torres M, Mancheno J M, de Las Rivas B, Munoz R: Tannin degradation by a novel tannase enzyme present in some Lactobacillus plantarum strains. Applied and environmental microbiology 2014, 80(10):2991-2997.
12. Garcia-Mazcorro J F, Suchodolski J S, Jones K R, Clark-Price S C, Dowd S E, Minamoto Y, Markel M, Steiner J M, Dossin 0: Effect of the proton pump inhibitor omeprazole on the gastrointestinal bacterial microbiota of healthy dogs. FEMS Microbiol Ecol 2012, 80(3):624-636.
13. Segata N, Waldron L, Ballarini A, Narasimhan V, Jousson O, Huttenhower C: Metagenomic microbial community profiling using unique clade-specific marker genes. Nat Methods 2012, 9(8):811-814.
14. Kanehisa M, Goto S, Sato Y, Furumichi M, Tanabe M: KEGG for integration and interpretation of large-scale molecular data sets. Nucleic acids research 2012, 40 (Database issue):D109-114.
15. Respondek F, Gerard P, Bossis M, Boschat L, Bruneau A, Rabot S, Wagner A, Martin J C: Short-chain fructo-oligosaccharides modulate intestinal microbiota and metabolic parameters of humanized gnotobiotic diet induced obesity mice. PLoS One 2013, 8(8):e71026.
16. Guo M, Zhao B, Liu H, Zhang L, Peng L, Qin L, Zhang Z, Li J, Cai C, Gao X: A Metabolomic Strategy to Screen the Prototype Components and Metabolites of Shuang-Huang-Lian Injection in Human Serum by Ultra Performance Liquid Chromatography Coupled with Quadrupole Time-of-Flight Mass Spectrometry. J Anal Methods Chem 2014, 2014:241505.

### Example 8: Characterization and Quantification of Systemic Metabolites from Sumac Tea

Several health benefits have been ascribed to gallotannins, a phytochemical abundant in the fruit sumac (1, 2). However, the bioavailability of gallotannins is limited due to its large size and complex structure; this is expected to lead to reduced health benefits.

However, gallotannins are considered hydrolysable because of a particular type of bond they possess, the depside ester linkage. Commercial esterases such as tannase, which is commercially used in several foods, have demonstrated gallotannin hydrolytic activity. Gallic acid is the hydrolytic product of gallotannins.

Previous studies describe the absorption of gallic acid from black tea, but the absorption from sumac tea has not previously been investigated (3). It is proposed that gallic acid, the smaller, hydrolytic product of gallotannin, is more absorbable, possibly through pre-treatment with a commercial, food-grade tannase. Improving the absorption of gallotannin may provide improved health benefits.

The dose proposed in this study is similar to the range of doses proposed in other studies. In a study with 200 mL 5% (w/v) black tea, 0.3 mmol of gallic acid was administered and an average of 2 umol/L was detected in plasma after 1.4 hours (3). Another study with 300 mL red wine contained 0.2 mmol gallic acid and an average of 0.05 umol/L was detected at 1.5 hrs (4). This study will administer 200 mL of a 4% (w/v) sumac tea treated with 0.2% tannase (w/v) to yields 3 mmol (566 mg/L) gallic acid available for absorption into blood circulation.

Toxicology of Gallic Acid: In rabbits, the oral LD₅₀ of both gallic acid and tannic acid is 2800 and 3400 mg/kg/day for 10 days (6). For a human equivalent of 75 kg, the equivalent dose would be 210,000 and 255,000 mg/day for 10 days. The highest selected dose proposed in this study is 566 mg/L and is 371-fold and 451-fold below the LD₅₀ dose.

Naturally occurring concentrations of gallotannins and gallic acid: Gallotannin and gallic acid naturally occur in many plant-based foodstuffs such as black and green tea, mangoes, berries, grapes, apples, tree nuts, beer, wine and sumac. In the dried fruits of sumac, gallotannin represents the major polyphenol, up to 22% or 220 mg/g (7). In a human clinical trial on diabetes, 3 g of sumac, 660 mg/g was administered daily for 120 days and no adverse effects were reported (8). The selected dose in this study, 566 mg of gallic acid, is nearly equivalent to the serving sizes used in previous studies.

Corresponding data from in vitro studies: In in-vitro studies, the effective dose to induce therapeutic effect is often higher than that administered in human clinical trials, though the in vitro study dosage is below the LD₅₀. For example, a 5000 mg/L tannin dosage produced a decrease in vascular smooth muscle cell migration in a transmembrane migration assay (2).

Study subjects consist of men and women ages 18-65 years with no history of chronic diseases, intestinal disorders, and no acute cardiac events, seizures, strokes, or cancer within the past six months as well as no recurrent hospitalizations (defined as 2 or more for any reason) within the past six months. Subjects will not have abused alcohol or other substances within the past six months, do not smoke more than one pack of cigarettes per week, do not exhibit liver or renal dysfunction and are not pregnant or lactating at the time of screening or any time during the study. Subjects should not have allergies to fruits, nuts, spices, herbs, or botanicals and should not have hepatitis B or C or HIV. Subjects performing more than 60 minutes of exercise 5 or more times per week will be excluded as will subjects consuming herbal-based or polyphenol-rich supplements of any kind. Subjects should not have used antibiotics within the past three months and should not have a history of dizziness or fainting during and/or after blood draws. For women ages 18-44 a urine pregnancy test will be performed during screening day and must be negative for the participant to continue in the study.

Number of participants: N=15 individuals are expected to be enrolled in order for 10 individuals to complete this pilot study considering screening failure and drop-outs. Each individual will consume all 3 study treatments with a 3-day washout in between. One treatment is consumed per study day and each study day is separated by a minimum of 3 days. Additional time in between study days, up to 14 days, will be permitted to accommodate participants' schedules. It will be randomized which study treatment participants will receive on study days 1, 2 and 3.

Study Treatments

The study treatments will be prepared using good manufacturing practices (GMP) and standard sanitation operating procedures (SSOPs) in Centeq Building A Rm 235. Each subject will be asked to consume a single dose (200 mL) of the study treatment on days 1, 2 and 3 of the experimental phase. Details on the preparation of the study treatment are explained under “Methods” below.

Study Overview: This a three-day study that compares gallotannin metabolite absorption from three different sumac tea samples prepared in 3 different ways. The study design is a randomized crossover clinical trial with a washout period of 3-14 days. Participants will consume each treatment on different days with a washout period included before the first treatment and in between the second and third treatments. The three treatments are pre-processed tea (treatment A), rapidly processed tea (treatment B) and unprocessed tea (treatment C) and each participant will be served 200 mL of tea.

A total of ten participants will be recruited. A screening visit and three study visits are proposed with dietary interventions taking place on each study day (Days 1, 2 and 3). Details on the experience of the subject are explained below.

Screening Visit: During the screening, the study coordinator will confirm inclusion and exclusion criteria with the participant based on self-reported information. The participant will be informed that deliberate misrepresentation of their health information could increase the risk of their participation in this study. Questions about hydration, food intake and difficulty with blood draws will also be collected before the study day.

During screening, participant's weight, height and age will be collected on a sample collection form. If participant is a woman of child-bearing age, between ages 18-44, a urine pregnancy test will be performed by qualified study personnel. If a positive pregnancy test result is received, participant will be excluded per exclusion criteria. This information will be recorded on the sample collection form viewed only by the study coordinator and PI. Participants who are eligible will move forward to study days 1, 2 and 3.

Blinding and Randomization: The study participants will be blind to which study treatment is administered, although the study coordinator will know which treatment is administered on study days 1, 2 and 3. Randomization will be performed using the RANDOM function in Microsoft Excel.

Days 1, 2 and 3: Before any study procedures, the study nurse or phlebotomist will perform a wellness check by asking the study participant about hydration, food intake and difficulty with blood draws. If study participant expresses concern about providing the blood sample, the study coordinator will ask the participant to drink an 8 oz glass of water and to lightly stretch to stimulate blood flow. If the study participant exhibits signs of severe nausea, dizziness, or anxiety towards blood draws, they will be withdrawn from this study without their consent (more details are described below under “15.0 Withdrawal of Participants” section).

Participants will be asked to consume 200 mL of 4% (w/v) sumac tea (unprocessed, rapidly processed or slowly processed). Blood samples (20 mL or 4 teaspoon each) will be collected right before consumption of the treatment and 1.5 hours after consumption of study treatment by the study nurse or study phlebotomist.

In case of a medical emergency, the following procedure will be implemented: first aid will be provided by study nurse or phlebotomist, emergency services will be contacted, the study PI and study physician will be notified. Follow-up will be conducted with the participant and reportable new information (RN I) will be reported to the institutional review board (IRB).

Primary Outcome: The primary endpoint of this study is to compare quantities of gallotannin metabolites, gallic acid specifically, detected from each of the three sumac tea study treatments. This information will show the optimal processing method that achieves the highest absorption of gallotannin from sumac tea.

Participant compensation: Participants will receive $25 per study day and a $50 bonus if they complete the study. The maximum compensation is $125. Payment will be received in the form of a one-time check after the completion of the subject's participation at the end of the study by mail.

An initial online survey for participants will take 5-10 minutes to complete. Participants will then be required to attend a familiarization session and receive information about consent; this is scheduled to be about one hour. This will be followed by a 20 minute screening visit and three separate study days as previously described, each expecting to require from 2 to 2.5 h of time.

Methods

Study Treatment Preparation: The study treatments will be prepared as follows: tea will be prepared using good manufacturing practices (GMP) and standard sanitation operating procedures (SSOPs). Treatment 1 is the pre-processed sumac tea and it will be prepared as follows: 500 mL of bottled drinking water will be heated to boiling temperature. Sumac powder will be added to make a pasteurized sumac tea at 8% (w/v) concentration. Tea will be steeped for 30 minutes before filtration through a commercial coffee filter. Tea will be cooled to room temperature after 1 hour. Next, the pasteurized sumac tea will be diluted with an equal volume of tannase enzyme solution (500 mL, 0.2%). The tannase enzyme solution will be made with bottled drinking water and added to the 500 mL pasteurized sumac tea. The final concentration of sumac in tea is 4% and final concentration of tannase in tea is 0.1% Tea will not be served until after 120 minutes as this is the minimum processing time required to completely hydrolyze gallotannins and be reduced as nonfunctional in the tea. The serving size will be 200 mL aliquots of pre-processed tea at room temperature.

Treatment 2 is the rapidly-processed sumac tea treatment will be prepared exactly as the pre-processed tea treatment with one exception. The 500 mL 0.2% tannase solution will not be added to the sumac tea until 1 to 2 minutes before serving. The serving size will be 200 mL, 100 mL of sumac tea and 100 mL of 0.2% tannase solution at room temperature.

Treatment 3 is the unprocessed sumac tea treatment will be prepared exactly as above but with no addition of tannase enzyme solution. Instead, the unprocessed sumac tea will be diluted with an equal volume of bottled drinking water. The serving size will be 200 mL, 100 mL of unprocessed tea and 100 mL of bottled drinking water at room temperature.

After serving, any extra tea or tannase solution will be discarded and a new study treatment will be prepared following the same formula. preparation will begin for new participants.

The details of study days in which samples are collected are as follows: on day 1 of the study, each participant will consume 1 serving of study treatment A. After a washout period 3-14 days, day 2 of the study will be performed. On day 2, the study participants will consume a different study treatment decided at random (B). After a washout period of 3-14 days, Day 3, the final day of the study will be performed in the same manner as days 1 and 2 except with a different study treatment (C). Study participants will consume all 3 of the study treatments (A, B, and C) by the end of the study and will have provided a baseline blood sample and another blood sample 1.5 hrs post consumption.

Each participant will be monitored by study personnel to ensure that the entire study treatment is consumed and to ensure no extraordinary or unsafe reactions are observed. Because study personnel will be present with participants for the duration of the treatment consumption and blood sample collection, the probably of risk will be reduced.

Sample handling Plasma samples will be aliquoted and stored in a −80° C. freezer until analysis. Plasma samples will be acidified with 50 μL/mL 88% formic acid before storage to ensure analyte stability. Plasma will be extracted with methanol and plasma extracts will be analyzed with a triple quadrupole mass spectrometer.

Determination of gallic acid in plasma: Gradient elution at 0.4 mL/min with phase A (0.1% formic acid water) and phase B (0.1% formic acid MeOH) will initially begin at 90% A and decrease to 60% A in 5 minutes, 5% in 7 minutes, return to initial conditions in 8 minutes and held at these conditions for 12 minutes. Separation will be achieved with a Kinetex C18 column (150×2 mm, 4 μm) The mass detector will operate in multiple reaction monitoring (MRM) mode to detect ions within a m/z of 100-300. Sumac gallotannin metabolites, namely gallic acid, will be analyzed in plasma as part of the tea processing method evaluation.

Minimization of Risk

Those with a history of fainting or nausea associated with blood draws will not be included in the study. Blood draws will be performed by a research nurse or phlebotomist, who will check the overall wellness status of participants before each blood draw. Participants are asked to drink water and eat a well-balanced meal the evening before study days. Finally, data will be kept confidential and will be encrypted. Data and specimen will be only handled by study personnel. Participant data and specimen will be encrypted with a number or code and these identifiers are not included in any data sheets or on any specimen samples.

Blood samples will be de-identified and stored at −80° C. until chemical analysis. Samples will be destroyed after the sponsor receives the final report and a manuscript has been accepted for publication (and it is clear that no internal or external re-analysis will be required). Samples will not be stored for additional future analysis.

Provisions to Ensure Safety of Participants

PI and study personnel will review any unexpected reportable events to determine any unexpected risk from this study (Data safety monitoring committee). Any reportable event will be reported to the study physician and the study will be immediately stopped in case of any serious adverse event.

Any study personnel will receive training by the study coordinator and principal investigator on how to comply with the Data Safety and Monitoring Plan.

Participants will be asked to contact the study coordinator or study nurse or phlebotomist if they feel the study treatments are causing any side effects. They will be asked about any side effect on each study day. Adverse events will be recorded on the adverse event form, which will be reviewed by the study physician to assess severity and causality. Events meeting the criteria for reporting to the IRB will be reported according to IRB policy.

The study coordinator will analyze data records weekly to ensure accuracy/security and compliance with the IRB-approved protocol.

Ranking of adverse events: For subjective adverse events (e.g., nausea, stomach pain, GI discomfort), participants will be asked to rank their experience on a scale from 1-10, where 1-3 will be ranked “minor”, 4-7 “moderate” and 8-10 “severe”. For objective adverse events (e.g., intestinal evacuation, clinical biomarkers), the clinical standard of care reference values will be used to rank the event by the study physician.

If more than 4 adverse events in the same category have been rated “moderate” or “severe” by the study physician, the study will be paused and continued after coordination with the study physician and IRB.

Withdrawal of Participants: If any participant exhibits signs of severe nausea, dizziness, or anxiety towards blood draws, they will be withdrawn from this study without their consent. They still will be compensated for the entire study day on which they are withdrawn.

Participants can withdraw from the research at any time without experiencing any disadvantage.

Recruitment and Consent

Recruitment will happen using the Texas A&M University mass email and a pre-designed advertisement. Participants who are interested will be asked to fill out an online pre-screening survey. Based on their response, participants will be invited to participate in a familiarization session. During this session, study information, rationale, design, schedule, the risks, and a descriptive presentation of participation will be given. Those who consent to participate in this study will go over the inclusion and exclusion criteria with the study coordinator. Qualified individuals will be contacted by a member of the study personnel, who will schedule the screening day. Being part of this study while pregnant or nursing may expose the unborn or nursing child to not yet evaluated risk factors, some of which may be currently unforeseeable. Therefore, pregnant and nursing women will be excluded from the study. If the participant is a woman of child-bearing age, 18-44 years, and able to become pregnant, a urine pregnancy test will be performed during screening and test must be negative before the participant can continue in the study. If female participants become aware of pregnancy during study, the pregnancy must be reported and participant will be terminated from study.

During the familiarization session, study information, rationale, design, schedule, the risks, and a descriptive presentation of participation will be given and each section of the informed consent form discussed. After familiarization session, participants can ask questions about the informed consent. Those who consent to participate in this study will go over the inclusion and exclusion criteria with the study coordinator. Qualified individuals will be contacted by a member of the study personnel, who will schedule their first study session.

The consent process will take place in a conference room or in a private meeting room with the study coordinator. Interested individuals can ask questions for as long as they feel necessary. Individuals do not have to sign the consent form right away and may take it home before consenting.

Participants will be reminded on study days that they do not have to complete the study and can discontinue the study at any time.

Study coordinators will not exert any coercion during the consent process and will ensure that participants understand any potential risk involved.

### References for Example 8

1. Perchellet J P, Gali H U, Perchellet E M, Klish D S, Armbrust A D. Antitumor-promoting activities of tannic acid, ellagic acid, and several gallic acid derivatives in mouse skin. Basic Life Sci. 1992; 59:783-801. Epub 1992/01/01. PubMed PMID: 1417700.
2. Zargham H, Zargham R. Tannin extracted from Sumac inhibits vascular smooth muscle cell migration. McGill journal of medicine: MJM: an international forum for the advancement of medical sciences by students. 2008; 11(2):119-23. PubMed PMID: 19148309.
3. Shahrzad S, Aoyagi K, Winter A, Koyama A, Bitsch I. Pharmacokinetics of gallic acid and its relative bioavailability from tea in healthy humans. J Nutr. 2001; 131(4):1207-10. Epub 2001/04104. doi: 10.1093/jn/131.4.1207. PubMed PMID: 11285327.
4. Cartron E, Fouret G, Carbonneau M-A, Lauret C, Michel F, Monnier L, Descomps B, Leger C L. Red-wine beneficial long-term effect on lipids but not on antioxidant characteristics in plasma in a study comparing three types of wine—description of two O-methylated derivatives of gallic acid in humans. Free Radical Res. 2003; 37(9):1021-35.
5. Henning S M, Wang P, Abgaryan N, Vicinanza R, de Oliveira D M, Zhang Y, Lee R P, Carpenter C L, Aronson W J, Heber D. Phenolic acid concentrations in plasma and urine from men consuming green or black tea and potential chemopreventive properties for colon cancer. Mol Nutr Food Res. 2013; 57(3):483-93. doi: 10.1002/mnfr.201200646. PubMed PMID: 23319439; PMCID: PMC3600069.
6. Dollahite J W, Pigeon R F, Camp B J. The toxicity of gallic acid, pyrogallol, tannic acid, and Quercus havardi in the rabbit. Am J Vet Res. 1962; 23:1264-7. PubMed PMID: 14028469.
7. Sumach. (Rhus Coriaria, L.). Bulletin of Miscellaneous Information (Royal Botanic Gardens, Kew). 1895; 1895(107):293-6. doi: 10.2307/4118504.
8. Shidfar F, Rahideh S T, Rajab A, Khandozi N, Hosseini S, Shidfar S, Mojab F. The effect of Sumac Rhuscoriaria L. Powder on serum glycemic status, ApoB, ApoA-I and total antioxidant capacity in type 2 diabetic patients. Iranian Journal of Pharmaceutical Research. 2014; 13(4):1249-55.

What is claimed is:

1. A pharmaceutical or nutraceutical formulation for improving an ability to process dietary tannins in a subject in need thereof, the formulation comprising an effective amount of a tannin-specific probiotic strain and an acid-tolerant tannase.

2. The pharmaceutical or nutraceutical formulation of claim 1, wherein the formulation increases intestinal free gallic acid concentration by at least 50% between about 2 hours and about 4 hours of administering the formulation to the subject.

3. The pharmaceutical or nutraceutical formulation of claim 1, wherein the tannin-specific probiotic strain comprises Lactobacillus plantarum, Lactococcus lactis, Enterococcus faecium, Enterobacter aerogenes, Streptococcus gallolyticus, Eubacterium oxidoreducens, or a combination thereof.

4. The pharmaceutical or nutraceutical formulation of claim 1, wherein the acid-tolerant tannase is sourced from an Ascochyta species, an Aspergillus species, a Penicillium species, a Fusarium species, a Trichoderma species, a Bacillus species, a Corynebacterium species, a Lactobacillus species, a Streptococcus species, a Klebsiella species, or a combination thereof.

5. The pharmaceutical or nutraceutical formulation of claim 1, wherein the ratio (w/w) of tannin-specific probiotic strain to the acid-tolerant tannase is from about 1:1000 to about 100:1.

6. The pharmaceutical or nutraceutical formulation of claim 1, further comprising at least one source of hydrolyzable tannins.

7. The pharmaceutical or nutraceutical formulation of claim 6, wherein the at least one source of hydrolyzable tannins comprises mango, amla, sumac, raspberries, blackberries, blueberries, strawberries, pomegranate, cloudberry, dates, grapefruit, banana, quince, sea buckthorn, apple, grapes, grape seeds, olive, currants, persimmon, gooseberry, cherry, kiwi, avocado, sumac, tea (such as green, oolong, white, black), sage, marjoram, oregano, cloves, chicory, oak, chamomile, peppermint, chestnut, soybeans, walnuts, pecans, walnut, lentils, broad beans, hazelnut, pistachio, and almond, or extracts thereof.

8. The pharmaceutical or nutraceutical formulation of claim 7, wherein the ratio (w/w) of the source of hydrolyzable tannin to the acid-tolerant tannase is from about 1:1000 to about 100:1.

9. The pharmaceutical or nutraceutical formulation of claim 1, wherein improving the ability to process dietary tannins comprises improving the subject's ability to hydrolyze dietary tannins, improving the subject's ability to absorb dietary tannins, improving the subject's ability to metabolize dietary tannins, increasing a urine level of a tannin metabolite in the subject, increasing a fecal level of a tannin metabolite in the subject, increasing a blood level of a tannin metabolite in the subject, or a combination thereof.

10. The pharmaceutical or nutraceutical formulation of claim 9, wherein improving the ability to process dietary tannins comprises an improvement of at least 30%.

11. The pharmaceutical or nutraceutical formulation claim 6, wherein the at least one source of hydrolyzable tannins is provided separately from the tannin-specific probiotic strain and the acid-tolerant tannase.

12. The pharmaceutical or nutraceutical formulation of claim 6, wherein the at least one source of hydrolyzable tannins is provided in a single dosage form with the tannin-specific probiotic strain and the acid-tolerant tannase.

13. The pharmaceutical or nutraceutical formulation of claim 1, further comprising a prebiotic.

14. The pharmaceutical or nutraceutical formulation of claim 13, wherein the prebiotic comprises a fructooligosaccharide, inulin, a galactooligosaccharide, resistant starch, pectin, a β-glucan, a xylooligosaccharide, a mucopolysaccharide, an isomaltooligosaccharide, an araganogalactan, a cellulose ether, a water-soluble hemicellulose, an alginate, agar, carrageenan, psyllium, guar gum, gum tragacanth, gum karaya, gum ghatti, gum acacia, gum arabic, a combination thereof, or a partially-hydrolyzed product thereof.

15. A multi-layer tablet comprising:
a. a core comprising a tannin-specific probiotic strain; and
b. a first layer surrounding the core, wherein the first layer comprises an acid-tolerant tannase.

16. The multi-layer tablet of claim 15, the core further comprising a prebiotic.

17. The multi-layer tablet of claim 15, the first layer further comprising a hydrolyzable tannin.

18. The multi-layer tablet of claim 15, further comprising an outer layer surrounding the first layer, wherein the outer layer comprises a controlled-release coating.

19. The multi-layer tablet of claim 15, wherein the ratio (w/w) of tannin-specific probiotic strain to the acid-tolerant tannase is from about 1:1000 to about 100:1.

20. The multi-layer tablet of claim 17, wherein the ratio (w/w) of the hydrolyzable tannin to the acid-tolerant tannase is from about 1:1000 to about 100:1.
